(19) United States
(12) Patent Application Publication
(10) Pub. No.: US 2026/0146106 A1
(43) Pub. Date: May 28, 2026

(54) BIFUNCTIONAL DEGRADERS OF GALACTOSE-DEFICIENT IMMUNOGLOBULINS

(71) Applicant: Biohaven Therapeutics Ltd., New Haven, CT (US)

(72) Inventors: Gene M. DUBOWCHIK, New Haven, CT (US); David A. SPIEGEL, Hamden, CT (US); Reese M. CALDWELL, Cambridge, MA (US); Pamela A. TRAIL, New Haven, CT (US)

(21) Appl. No.: 19/449,561

(22) Filed: Jan. 15, 2026

Related U.S. Application Data
(63) Continuation of application No. 18/548,113, now abandoned.
(60) Provisional application No. 63/159,383, filed on Mar. 10, 2021.

(51) Int. Cl. C07K 16/44; A61K 39/00; A61K 47/54; C07K 19/00

(52) U.S. Cl. CPC C07K 16/44; A61K 47/549; C07K 19/00; A61K 2039/505

(57) ABSTRACT

An agent including a glycan-specific IgG antibody moiety, a cellular receptor binding moiety which binds to hepatocytes or other degrading cells through asialoglycoprotein (ASGPR) receptors of hepatocytes or other cell receptors which are on the surface degrading cells in a patient or subject, and optionally, a linker moiety connecting the glycan-specific IgG antibody moiety and the cellular receptor binding moiety.

### CROSS-REFERENCE TO RELATED APPLICATION

This application is a continuation of U.S. patent application Ser. No. 18/548,113 filed Aug. 28, 2023, which claims priority to U.S. Provisional Application No. 63/159,383 filed Mar. 10, 2021, and all the benefits accruing therefrom under 35 U.S.C. § 119 and 35 U.S.C. § 120, the contents of which applications are incorporated herein in their entireties by reference.

### FIELD OF THE INVENTION

The present invention relates to bifunctional molecules which contain a circulating protein binding moiety linked through a linker group to a cellular receptor binding moiety. Specifically, the present invention relates to bifunctional molecules containing a circulating protein binding moiety that binds to galactose-deficient immunoglobulins.

### BACKGROUND OF THE INVENTION

IgA nephropathy (IgAN) is an autoimmune disease characterized by circulating nephritogenic immune complexes composed of galactose-deficient IgA1 (Gd-IgAQ1) and a glycan-specific IgG antibody. These immune complexes accumulate in the glomerular mesangium and induce the mesangioproliferative glomerulonephritis characteristic of IgAN, a cause of kidney injury. The hypothesis is supported by the findings that, in renal immunodeposits of IgA neurophathy patients, IgG is enriched for Gd-IgAQ1-specific antibodies. Moreover, serum levels of Gd-IgA1 (autoantigen) and the corresponding autoantibodies were each found to correlate with IgAN severity and progression. Suzuki et al. “Aberrantly glycosylated IgA1 in IgA nephropathy patients is recognized by IgG antibodies with restricted heterogeneity” J. Clin. Invest. 2009, 119(6), 1668-1677 (Appendix A), which is incorporated herein in its entirety by reference.

A recent study provided in vivo evidence for the nephritogenic role of the IgG autoantibodies in IgAN. The study showed that immune complexes formed from human IgG autoantibody and Gd-IgA1 injected intravenously into mice produced glomerular injury. The histopathological changes in the injured tissues were characteristic of IgAN. Exploratory kidney-transcriptome profiling indicated that these immune complexes altered gene expression of multiple pathways consistent with the changes observed in kidney biopsies of patients with IgAN. Moldoveanu et al. “Experimental evidence of pathogenic role of IgG autoantibodies in IgG nephropathy” J. Autoimmun. 2021, 118, 102593 (Appendix B), which is incorporated herein in its entirety by reference.

IgAN is the most common form of primary glomerulonephritis in the world that currently has no treatment. Thus, there remains a need in new medicines capable of treating or slowing down progression of the disease.

### SUMMARY OF THE INVENTION

The present invention is directed to bifunctional molecules capable of binding and degrading galactose-deficient immunoglobulins.

In an embodiment, provided an agent including:
a glycan-specific IgG antibody moiety,
a cellular receptor binding moiety which binds to hepatocytes or other degrading cells through asialoglycoprotein (ASGPR) receptors of hepatocytes or other cell receptors which are on the surface degrading cells in a patient or subject, and
optionally a linker moiety connecting the glycan-specific IgG antibody moiety and the cellular receptor binding moiety.

In another embodiment, provided is a method of removing galactose-deficient IgA1 in a patient or subject in need by administering to the mammal the agent.

In another embodiment, provided is a method of treating a disease state and/or condition which is associated with the upregulation of galactose-deficient IgA1 in a patient or subject in need by administering to the mammal an effective amount of the agent.

In another embodiment, provided is A composition including the agent and at least one additional agent comprising a moiety capable of binding to the antibody that forms the antibody moiety of the first compound.

### DETAILED DESCRIPTION OF THE INVENTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

Additional aspects will be set forth in part in the description which follows and, in part, will be apparent from the description.

In an embodiment, provided is an agent including:
an antibody binding moiety,
a cellular receptor binding moiety which binds to hepatocytes or other degrading cells through asialoglycoprotein (ASGPR) receptors of hepatocytes or other cell receptors which are on the surface degrading cells in a patient or subject, and
optionally a linker moiety linking the antibody moiety and the cellular receptor binding moiety.

The antibody binding moiety may be a glycan-specific IgG antibody moiety. The glycan-specific IgG antibody moiety may be configured to bind to a galactose-deficient IgA1 (Gd-IgAQ1). In an embodiment, the glycan-specific IgG antibody moiety may be a recombinant IgG antibody moiety (rIgG). In another embodiment, the glycan-specific IgG antibody moiety may be isolated from sera of a IgA nephropathy patient.

The presently claimed bifunctional compounds selectively bind to a galactose-deficient IgA1 in circulation and form a protein complex that then binds a cellular receptor and is endocytosed and degraded. As a consequence of this mechanism, the galactose-deficient IgA1 is eliminated from circulation by hepatocytes, macrophages, or another cell type, thus resulting in lowered level of the galactose-deficient IgA1 with the potential of attenuating the IgAN symptoms. In certain instances, the galactose-deficient IgA1 may be eliminated, resulting in substantially reduced symptoms or even a cure or elimination of IgAN.

In some embodiments, the present disclosure provides agents having a structure of:

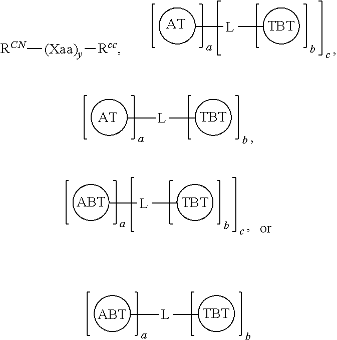

or a salt thereof. In some embodiments, an agent has the structure of

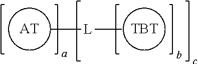

or a salt thereof. In some embodiments, an agent has the structure of

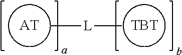

or a salt thereof. In some embodiments, an agent has the structure of

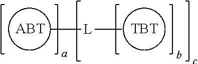

or a salt thereof. In some embodiments, an agent has the structure of

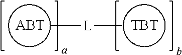

or a salt thereof. In some embodiments, each cellular receptor binding moiety independently has the structure of —(R^{CN}-(Xaa)y-R^{CC}) or salt form thereof. In the above structures, a, b, and c may independently be an integer of 1 or greater.

In some embodiments, an antibody binding moiety is a universal antibody binding moiety.

In some embodiments, an antibody binding moiety comprises one or more amino acid residues. In some embodiments, an antibody binding moiety is or comprises a peptide moiety. In some embodiments, an antibody binding moiety is or comprises a cyclic peptide moiety. In some embodiments, such antibody binding moiety comprises one or more natural amino acid residues. In some embodiments, such antibody binding moiety comprises one or more unnatural natural amino acid residues.

In some embodiments, an antibody-binding moiety is a cyclic peptide moiety. In some embodiments, an antibody binding moiety is or comprises

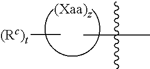

or a salt form thereof. In some embodiments, an antibody binding moiety is

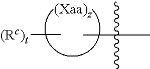

or a salt form thereof. In some embodiments, each antibody binding moiety in an agent independently is or comprises

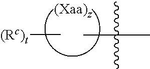

or a salt form thereof. In some embodiments, each antibody binding moiety in an agent is independently

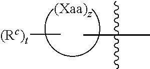

or a salt form thereof. In some embodiments, each antibody binding moiety in an agent is of the same antibody binding moiety or a salt thereof.

In some embodiments, the present disclosure provides a compound of formula I-a:

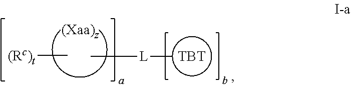

or a salt thereof, wherein:
each Xaa is independently a residue of an amino acid or an amino acid analog;
t is 0-50;
z is 1-50;
L is a linker moiety;
TBT is a cellular receptor binding moiety;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a is 1. In some embodiments, b is 3. In some embodiments, a is 1 and b is 3, and a compound of formula I-a has the structure of

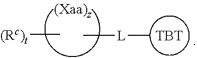

In some embodiments, each residue, e.g., Xaa, is independently a residue of an amino acid or an amino acid analog, wherein the amino acid or the amino acid analog has the structure of H-L^{a1}-L^{a1}-C(R^{a2})(R^{a3})-L^{a2}-L^{a2}-H or a salt thereof. In some embodiments, an amino acid has the structure of NH(R^{a1})-L^{a1}-C(R^{a2})(R^{a3})-L^{a2}-COOH or a salt thereof. In some embodiments, an amino acid analog has the structure of H-L^{a1}-L^{a1}-C(R^{a2})(R^{a3})-L^{a2}-L^{a2}-H or a salt thereof. In some embodiments, in such an amino acid analog, the first -L^{a1}- (bonded to —H in the formula) is not —N(R^{a1})— (e.g., is optionally substituted bivalent C₁₋₆ aliphatic). In some embodiments, in H-L^{a1}-L^{a1}-, -L^{a1}-L^{a1}- bonds to the —H through an atom that is not nitrogen. In some embodiments, in -L^{a2}-L^{a2}-H, -L^{a2}L^{a2}- is not bonded to the —H through —C(O)O—. In some embodiments, each residue, e.g., each Xaa in formula I-a, is independently a residue of an amino acid having the structure of formula A-I.

In some embodiments, each Xaa independently has the structure of -L^{a1}-L^{a1}-C(R^{a2})(R^{a3})-L^{a2}-L^{a2}-. In some embodiments, each Xaa independently has the structure of -L^{ax1}-L^{a1}-C(R^{a2})(R^{a3})-L^{a2}-L^{ax2}- wherein L^{ax1} is optionally substituted —NH—, optionally substituted —CH₂—, —N(R^{a1})—, or —S—, L^{ax2} is optionally substituted —NH—, optionally substituted —CH₂—, —N(R^{a1})—, or —S—, and each other variable is independently as described herein. In some embodiments, L^{ax1} is optionally substituted —NH—, or —N(R^{a1})—. In some embodiments, L^{ax1} is optionally substituted —CH₂—, or —S—. In some embodiments, L^{ax2} is optionally substituted —NH—, optionally substituted —CH₂—, —N(R^{a1})—, or —S—.

In some embodiments, optionally substituted —CH₂— is —C(O)—. In some embodiments, optionally substituted —CH₂— is not —C(O)—. In some embodiments, L^{ax2} is —C(O)—. In some embodiments, each Xaa independently has the structure of —N(R^{a1})-L^{a1}-C(R^{a2})(R^{a3})-L^{a2}-CO—.

In many embodiments, two or more residues, e.g., two or more Xaa residues, are linked together such that one or more cyclic structures are formed. Residues can be linked, optionally through a linker (e.g., L^{T}) at any suitable positions. For example, a linkage between two residues can connect each residue independently at its N-terminus, C-terminus, a point on the backbone, or a point on a side chain, etc. In some embodiments, two or more side chains of residues, e.g., in compounds of formula I-a, (e.g., R^{a2} or R^{a3} of one amino acid residue with R^{a2} or R^{a3} of another amino acid residue) are optionally take together to form a bridge, e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-a through a side chain of an amino acid residue (e.g., Xaa in formula I-a). In some embodiments, such a side chain comprises an amino group or a carboxylic acid group. In some embodiments, L^{T} is L^{b} as described herein. In some embodiments, a linkage, e.g., L^{b} or L^{T}, connects a side chain with a N-terminus or a C-terminus of a residue. In some embodiments, a linkage connects a side chain with an amino group of a residue. In some embodiments, a linkage connects a side chain with an alpha-amino group of a residue. In some embodiments, as illustrated herein, a linkage, e.g., L^{b} or L^{T}, is —CH₂—C(O)—. In some embodiments, the —CH₂— is bonded to a side chain, e.g., bound to —S— of a cysteine residue, and the —C(O)— is bonded to an amino group, e.g., an alpha-amino group of a residue. In some embodiments, a linkage, e.g., L^{b} or L^{T}, is optionally substituted —CH₂—S—CH₂—C(O)—NH—, wherein each end is bonded to the alpha-carbon of a residue. In some embodiments, the —NH— is of an alpha-amino group of a residue, e.g., of a N-terminal residue.

In some embodiments,

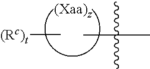

is an antibody binding moiety

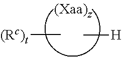

binds to an antibody). In some embodiments,

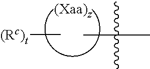

is a universal antibody binding moiety. In some embodiments,

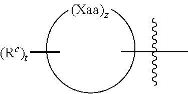

is a universal antibody binding moiety which can bind to antibodies having different Fab regions. In some embodiments,

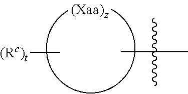

is a universal antibody binding moiety that can bind to a Fc region. In some embodiments, an antibody binding moiety, e.g., a universal antibody binding moiety having the structure of

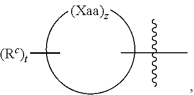

can bind to a Fc region bound to an Fc receptor. In some embodiments, an antibody binding moiety, e.g., of an antibody binding moiety having the structure of

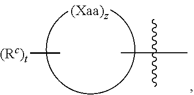

has the structure of

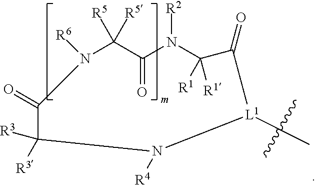

In some embodiments,

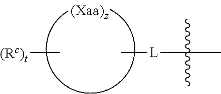

has the structure of

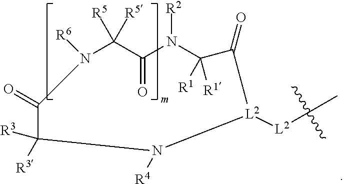

In certain embodiments, the present disclosure provides a compound of formula II:

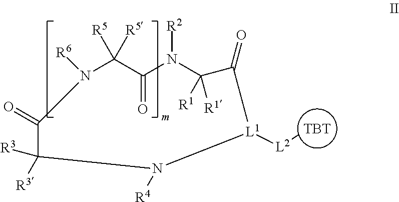

or a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety that connects

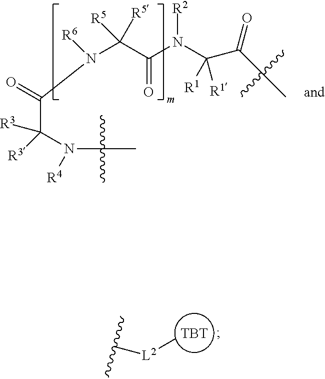

L² is a covalent bond or a C₁₋₃₀ optionally substituted bivalent straight or branched saturated or unsaturated hydrocarbon chain wherein 1-10 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—, —(CH₂OCH₂)n-, —(OCH₂CH₂)n-, or -Cy¹-, wherein each -Cy¹- is independently a 5-6 membered heteroarylenyl with 1-4 heteroatoms independently selected from nitrogen, oxygen or sulfur;
TBT is a cellular receptor binding moiety; and
each of m and n is independently 1, 2, 3, 4, 5, 6, 7, 8, 9, or 10.

In some embodiments, an amino acid has the structure of formula A-I:

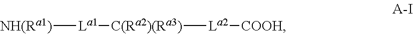

or a salt thereof, wherein:
   each of R^{a1}, R^{a2}, R^{a3} is independently -L^{a}-R′;
   each of L^{a1} and L^{a2} is independently L^{a};
   each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
   each R′ is independently —R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
   two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a residue has the structure of —N(R^{a1})-L^{a1}-C(R²)(R^{a3})-L^{a2}-COO— or a salt form thereof.

In some embodiments, an amino acid analog is a compound in which the amino group and/or carboxylic acid group are independently replaced with an optionally substituted aliphatic or heteroaliphatic moiety. As those skilled in the art will appreciate, many amino acid analogs, which mimics structures, properties and/or functions of amino acids, are described in the art and can be utilized in accordance with the present disclosure, e.g., in various moieties. In some embodiments, one or more peptide groups are optionally and independently replaced with non-peptide groups. In some embodiments, an amino acid moiety in a polypeptide or peptide is replaced with an amino acid analog moiety.

### Mates

In some embodiments, the present disclosure provides an agent comprising:
an antibody moiety,
a cellular receptor binding moiety, and
optionally a linker moiety linking an antibody moiety and a cellular receptor binding moiety.

Such an agent may be referred to as a MATE agent or MATE. The MATE agents are described, for example, in International Application No. PCT/US2020/061127 filed Nov. 18, 2020 and U.S. Provisional Application No. 63/146,584 filed Feb. 6, 2021, the content of each which application is incorporated herein in its entirety by reference. In some embodiments, an agent comprises an antibody moiety, a cellular receptor binding moiety, and a linker moiety linking an antibody moiety and a cellular receptor binding moiety.

The antibody binding moiety may be a glycan-specific IgG antibody moiety. The glycan-specific IgG antibody moiety may be configured to bind to a galactose-deficient IgA1 (Gd-IgA1). In an embodiment, the glycan-specific IgG antibody moiety may be a recombinant IgG antibody moiety (rIgG). In another embodiment, the glycan-specific IgG antibody moiety may be isolated from sera of a IgA nephropathy patient.

In some embodiments, an agent has the structure of formula M-L:

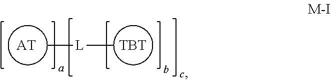

or a pharmaceutically acceptable salt thereof, wherein:
   each of a, b and c is independently 1-200;
   each AT is independently an antibody moiety;
   L is a linker moiety; and
   each TBT is independently a cellular receptor binding moiety.

In some embodiments, an agent has the structure of formula M-II:

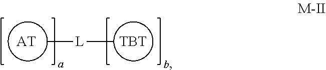

or a pharmaceutically acceptable salt thereof, wherein:
   each of a and b is independently 1-200;
   each AT is independently an antibody moiety;
   L is a linker moiety; and
   each TBT is independently a cellular receptor binding moiety.

In some embodiments, an agent comprises one and no more than one antibody moiety. In some embodiments, one or no more than one antibody moiety is bound to a linker moiety. In some embodiments, a is 1. In some embodiments, an agent comprises two or more antibody moieties. In some embodiments, two or more antibody moieties are bound to a single linker moiety. In some embodiments, a is 2 or more. In some embodiments, one and no more than one cellular receptor binding moiety is bonded to a linker moiety. In some embodiments, b is 1. In some embodiments, two or more cellular receptor binding moiety is bonded to a single linker moiety. In some embodiments, b is 2 or more. In some embodiments, an agent comprises one and no more than one cellular receptor binding moiety. In some embodiments, c is 1. In some embodiments, b is 1 and c is 1. In some embodiments, a is 1, b is 1 and c is 1. In some embodiments, an agent comprises two or more target binding moieties. In some embodiments, b is 2 or more and c is 1. In some embodiments, b is 2 or more and c is 2 or more. In some embodiments, b is 1 and c is 2 or more.

In some embodiments, c is 1-20, e.g., 1, 2, 3, 4, 5, 6, 7, 8, 9, 10, 11, 12, 13, 14, 15, 16, 17, 18, 19, or 20. In some embodiments, c is 1-15. In some embodiments, c is 1-10. In some embodiments, c is 1-9. In some embodiments, c is 1-8. In some embodiments, c is 1-7. In some embodiments, c is 1-6. In some embodiments, c is 1-5. In some embodiments, c is 1-4. In some embodiments, c is 1-3. In some embodiments, c is 1-2. In some embodiments, c is 1. In some embodiments, c is 2. In some embodiments, c is 3. In some embodiments, c is 4. In some embodiments, c is 5. In some embodiments, c is 6. In some embodiments, c is 7. In some embodiments, c is 8. In some embodiments, c is 9.

In some embodiments, each cellular receptor binding moiety in an agent is the same. In some embodiments, each linker moiety connecting a cellular receptor binding moiety to an antibody moiety is the same. In some embodiments, TBT in an agent are the same. In some embodiments, -L-(TBT)_{b} are the same.

In some embodiments, b is 1. In some embodiments, c is 1. In some embodiments, c is two or more. In some embodiments, c is 2. Those skilled in the art appreciate that various technologies can be utilized to conjugate antibody moieties with target binding moieties (e.g., certain technologies utilized for preparing antibody-drug conjugates) in accordance with the present disclosure. In some embodiments, target binding moieties are connected to antibody moieties through certain types groups and/or amino acid residues. For example, in some embodiments, target binding moieties are connected to lysine residues optionally through linker moieties. In some embodiments, target binding moieties are connected to cysteine residues optionally through linker moieties. In some embodiments, target binding moieties are connected to unnatural amino acid residues optionally through linker moieties. In some embodiments, the present disclosure provides technologies for selectively linking target binding moieties to certain particular amino acid residues optionally through linker moieties. In some embodiments, provided technologies selectively connect target binding moieties to certain types of amino acid residues, e.g., lysine residues, optionally through linker moieties. In some embodiments, provided technologies selectively connect target binding moieties to particular sites of antibody moieties optionally through linker moieties. In some embodiments, provided technologies selectively connect target binding moieties to certain types of amino acid residues at particular sites optionally through linker moieties. For example, in some embodiments, target binding moieties are connected to K246 and K248 of an IgG1 heavy chain and amino acid residues corresponding thereto optionally through linker moieties. For example, in some embodiments, target binding moieties are connected to K251 and K253 of an IgG2 heavy chain and amino acid residues corresponding thereto optionally through linker moieties. For example, in some embodiments, target binding moieties are connected to K239 and K241 of an IgG4 heavy chain and amino acid residues corresponding thereto optionally through linker moieties. In some embodiments, a cellular receptor binding moiety is connected to a particular amino acid residue or site optionally through a linker. In some embodiments, each cellular receptor binding moiety is independently connected to a particular amino acid residue or site optionally through a linker. As appreciated by those skilled in the art, an antibody agent may comprise more than one particular sites (e.g., one on each of the more than one chain (e.g., one or each heavy chain)). In some embodiments, an antibody moiety comprise two heavy chains and one or both of the amino acid residues or amino acid residues corresponding thereto are each independently connected to a cellular receptor binding moiety optionally through a linker. In some embodiments, one and no more than one is connected. In some embodiments, c is 1. In some embodiments, both are connected. In some embodiments, c is 2. In some embodiments, both target binding moieties and/or both linker moieties (if any) are the same.

### Antibody Binding Moieties

Various antibody binding moieties including universal antibody binding moieties can be utilized in accordance with the present disclosure. Certain antibody binding moieties and technologies for identifying and/or assessing antibody binding moieties are described in WO2019/023501 and WO2019/136442, each of which is incorporated herein in its entirety by reference. Those skilled in the art appreciates that additional technologies in the art may be suitable for identifying and/or assessing antibody binding moieties in accordance with the present disclosure. In some embodiments, an antibody binding moiety comprises one or more amino acid residues, each independently natural or unnatural.

In some embodiments, an antibody binding moiety, e.g., a protein binding moiety (e.g., an antibody binding moiety (e.g., a universal antibody binding moiety)), has the structure of

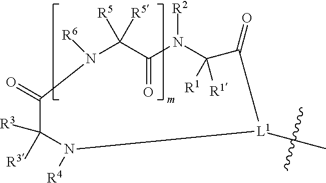

or a salt form thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety; and
each of m and n is independently 1, 2, 3, 4, 5, 6, 7, 8, 9, 10, 11, 12, 13, 14, 15, 16, 17, 18, 19, or 20.

In some embodiments, L¹ is an optionally substituted trivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments L¹ is —(CH₂CH₂O)₂₋₄— or —(CH₂CH₂O)₂—.

In some embodiments, an antibody binding moiety, e.g. a protein binding moiety (e.g., an antibody binding moiety (e.g., a universal antibody binding moiety)), has the structure of

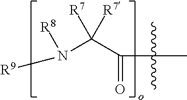

or a salt form thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{7′} is independently hydrogen or optionally substituted C₁₋₃ aliphatic;
each of R⁸ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur; and
   R⁹ is hydrogen, optionally substituted C₁₋₃ aliphatic, or —C(O)—.

In some embodiments, an antibody binding moiety, e.g., a universal antibody binding moiety is or comprises a peptide moiety, e.g., a moiety having the structure of R^{c}-(Xaa)z- or a salt form thereof, wherein each of R^{c}, z and Xaa is independently as described herein. In some embodiments, one or more Xaa are independently an unnatural amino acid residue. In some embodiments, side chains of two or more amino acid residues may be linked together to form bridges. For example, in some embodiments, side chains of two cysteine residues may form a disulfide bridge comprising —S—S— (which, as in many proteins, can be formed by two —SH groups).

In some embodiments, an antibody binding moiety, e.g. a protein binding moiety (e.g., an antibody binding moiety (e.g., a universal antibody binding moiety)), is or comprises a cyclic peptide moiety, e.g., a moiety having the structure of

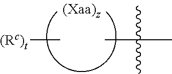

or a salt form thereof, wherein:
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a heteroatom is independently selected from oxygen, nitrogen, sulfur, phosphorus and silicon.

In some embodiments, an antibody binding moiety is or comprises R^{c}-(Xaa)z- or a salt form thereof, wherein each variable is as described herein. In some embodiments, a protein binding moiety is or comprises R^{c}-(Xaa)z- or a salt form thereof, wherein each variable is as described herein. In some embodiments, an antibody binding moiety, e.g., a universal antibody binding moiety, is or comprises R^{c}-(Xaa)z- or a salt form thereof, wherein each variable is as described herein. In some embodiments, an antibody binding moiety is or comprises

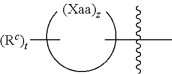

or a salt form thereof, wherein each variable is as described herein. In some embodiments, a protein binding moiety is or comprises

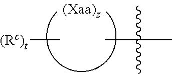

or a salt form thereof, wherein each variable is as described herein. In some embodiments, an antibody binding moiety, e.g., a universal antibody binding moiety, is or comprises

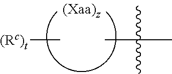

or a salt form thereof, wherein each variable is as described herein. In some embodiments, an antibody binding moiety, e.g., a universal antibody binding moiety is R^{c}-(Xaa)z- or

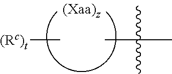

or a salt form thereof, and is or comprises a peptide unit. In some embodiments, -(Xaa)z- is or comprises a peptide unit. In some embodiments, amino acid residues may form bridges, e.g., connections formed by side chains optionally through linker moieties (e.g., L); for example, as in many polypeptides, cysteine residues may form disulfide bridges. In some embodiments, a peptide unit comprises an amino acid residue (e.g., at physiological pH about 7.4, “positively charged amino acid residue”, Xaa^{P}), e.g., a residue of an amino acid of formula A-I that has a positively charged side chain. In some embodiments, a peptide unit comprises R. In some embodiments, at least one Xaa is R. In some embodiments, a peptide unit is or comprises APAR. In some embodiments, a peptide unit is or comprises RAPA. In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{P}. In some embodiments, a peptide unit is or comprises Xaa^{P}Xaa^{P}XaaXaa^{A}. In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P}. In some embodiments, a peptide unit is or comprises Xaa^{P}Xaa^{P}Xaa^{A}Xaa^{A}Xaa^{P}. In some embodiments, a peptide unit is or comprises Xaa^{P}Xaa^{P}Xaa^{P}Xaa^{A}. In some embodiments, a peptide unit is or comprises two or more Xaa^{A}Xaa^{A}Xaa^{P}. In some embodiments, a peptide residue comprises one or more proline residues. In some embodiments, a peptide unit is or comprises HWRGWA. In some embodiments, a peptide unit is or comprises WGRR. In some embodiments, a peptide unit is or comprises RRGW. In some embodiments, a peptide unit is or comprises NKFRGKYK. In some embodiments, a peptide unit is or comprises NRFRGKYK. In some embodiments, a peptide unit is or comprises NARKFYK. In some embodiments, a peptide unit is or comprises NARKFYKG. In some embodiments, a peptide unit is or comprises HWRGWV. In some embodiments, a peptide unit is or comprises KHFRNKD. In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide unit comprises RHRFNKD. In some embodiments, a peptide unit is RHRFNKD. In some embodiments, a peptide unit comprises TY. In some embodiments, a peptide unit is TY. In some embodiments, a peptide unit comprises TYK. In some embodiments, a peptide unit is TYK. In some embodiments, a peptide unit comprises RTY. In some embodiments, a peptide unit is RTY. In some embodiments, a peptide unit comprises RTYK. In some embodiments, a peptide unit is RTYK. In some embodiments, a peptide unit is or comprises a sequence selected from PAM. In some embodiments, a peptide unit comprises WHL. In some embodiments, a peptide unit is WHL. In some embodiments, a peptide unit is or comprises WXL, wherein X is an amino acid residue as described herein, e.g., one suitable for connection with another moiety (e.g., an amino acid residue comprising —COOH or a salt or activated form thereof such as D, E, etc.). In some embodiments, a peptide unit comprises WDL. In some embodiments, a peptide unit is WDL. In some embodiments, a peptide unit comprises ELVW. In some embodiments, a peptide unit is ELVW. In some embodiments, a peptide unit comprises GELVW. In some embodiments, a peptide unit is GELVW. In some embodiments, a peptide unit is or comprises a sequence selected from AWHLGELVW. In some embodiments, a peptide unit is or comprises AWHLGELVW. In some embodiments, a peptide unit is or comprises a sequence selected from AWDLGELVW. In some embodiments, a peptide unit is or comprises AWDLGELVW. In some embodiments, a peptide unit is or comprises AWXLGELVW, wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DCAWHLGELVWCT, wherein the two cysteine residues can form a disulfide bond as found in natural proteins. In some embodiments, a peptide unit is or comprises DCAWHLGELVWCT, wherein the two cysteine residues can form a disulfide bond as found in natural proteins. In some embodiments, a peptide unit is or comprises a sequence selected from DCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises DCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, X comprises —COOH or a salt or activated form thereof in its side chain. In some embodiments, a peptide unit is or comprises a sequence selected from DCAWDLGELVWCT, wherein the two cysteine residues can form a disulfide bond as found in natural proteins. In some embodiments, a peptide unit is or comprises DCAWDLGELVWCT, wherein the two cysteine residues can form a disulfide bond as found in natural proteins. In some embodiments, a peptide unit is or comprises a sequence selected from Fc-III. In some embodiments, a peptide unit is or comprises Fc-III. In some embodiments, a peptide unit is or comprises a sequence selected from ^{D}P^{L}PAWXLGELVW, wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises ^{D}P^{L}PAWXLGELVW, wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from ^{D}P^{L}PAWDLGELVW. In some embodiments, a peptide unit is or comprises ^{D}P^{L}PAWDLGELVW. In some embodiments, a peptide unit is or comprises a sequence selected from ^{D}P^{L}PAWHLGELVW, wherein the two cysteine residues can form a disulfide bond as found in natural proteins. In some embodiments, a peptide unit is or comprises ^{D}P^{L}PAWHLGELVW (e.g., FcBP-1), wherein the two cysteine residues can form a disulfide bond as found in natural proteins. In some embodiments, a peptide unit is or comprises a sequence selected from FcBP-1. In some embodiments, a peptide unit is or comprises a sequence selected from ^{D}P^{L}PDCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises ^{D}P^{L}PDCAWXLGELVWCT, wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from ^{D}P^{L}PDCAWHLGELVWCT, wherein the two cysteine residues can form a disulfide bond as found in natural proteins. In some embodiments, a peptide unit is or comprises ^{D}P^{L}PDCAWHLGELVWCT (e.g., FcBP-2), wherein the two cysteine residues can form a disulfide bond as found in natural proteins. In some embodiments, a peptide unit is or comprises a sequence selected from ^{D}P^{L}PDCAWDLGELVWCT, wherein the two cysteine residues can form a disulfide bond as found in natural proteins. In some embodiments, a peptide unit is or comprises ^{D}P^{L}PDCAWDLGELVWCT, wherein the two cysteine residues can form a disulfide bond as found in natural proteins. In some embodiments, a peptide unit is or comprises a sequence selected from FcBP-2. In some embodiments, a peptide unit is or comprises a sequence selected from CDCAWXLGELVWCTC, wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises CDCAWXLGELVWCTC, wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from CDCAWHLGELVWCTC, wherein the first and the last cysteines, and the two cysteines in the middle of the sequence, can each independently form a disulfide bond as in natural proteins. In some embodiments, a peptide unit is or comprises CDCAWHLGELVWCTC, wherein the first and the last cysteines, and the two cysteines in the middle of the sequence, can each independently form a disulfide bond as in natural proteins. In some embodiments, a peptide unit is or comprises a sequence selected from CDCAWDLGELVWCTC, wherein the first and the last cysteines, and the two cysteines in the middle of the sequence, can each independently form a disulfide bond as in natural proteins. In some embodiments, a peptide unit is or comprises CDCAWDLGELVWCTC, wherein the first and the last cysteines, and the two cysteines in the middle of the sequence, can each independently form a disulfide bond as in natural proteins. In some embodiments, a peptide unit is or comprises a sequence selected from Fc-III-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus —COOH. It is appreciated by those skilled in the art that in various embodiments, when a peptide unit is connected to another moiety, an amino acid residue of a peptide unit may be connected through various positions, e.g., its backbone, its side chain, etc. In some embodiments, an amino acid residue is modified for connection. In some embodiments, an amino acid residue is replaced with another suitable residue for connection while maintaining one or more properties and/or activities a peptide unit (e.g., binding to an antibody as described herein). For example, in some embodiments, an amino acid residue is replaced with an amino acid residue with a side chain comprising —COOH or a salt or activated form thereof (e.g., side chain being —CH₂—COOH or a salt or activated form thereof). As exemplified herein, in various sequences H may be replaced with D (e.g., in various peptide units comprising WHL). In some embodiments, a peptide unit is connected to another moiety through —COOH or a salt or activated form thereof, e.g., through formation of e.g., —CON(R′)—. In some embodiments, R′ is —H. In some embodiments, —COOH is in a side chain of an amino acid residue. In some embodiments, in a sequence described herein (e.g., DCAWHLGELVWCT), 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, a peptide moiety is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues may independently and optionally form a disulfide bond. In some embodiments, the total number of replacements, deletions and insertions is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions.

In some embodiments, an antibody binding moiety comprises or has the structure of DCAWHLGELVWCT or a salt form thereof, wherein the two C residues are linked by a —S—S—. In some embodiments, an antibody binding moiety comprises or has the structure of DCAWHLGELVWCT or a salt form thereof, wherein the N-terminus is capped with R—C(O)—. In some embodiments, wherein R is methyl. In some embodiments, an antibody binding moiety is connected to the rest of a molecule through its C-terminus.

In some embodiments, -(Xaa)z- is or comprises [X¹]ₚ₁[X²]ₚ₂—X³X⁴X⁵X⁶X⁷X⁸X⁹X¹⁰X¹¹X¹²—[X¹³]ₚ₁₃—[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties described in Table A-1, Table 1, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

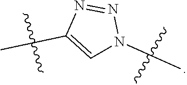 [SMILES: *c1cn(*)nn1]

In some embodiments, L^{a} is

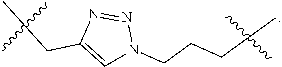 [SMILES: *CCCn1cc(C*)nn1]

In some embodiments, such an L^{a} can be formed by a —N₃ group of the side chain of one amino acid residue, and the of the [symbol] side chain of another amino acid residue. In some [symbol] embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

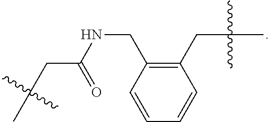 [SMILES: *CC(=O)NCc1ccccc1C*]

In some embodiments, L^{a} is

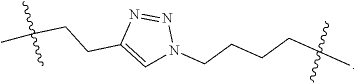 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z-is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X⁵ is

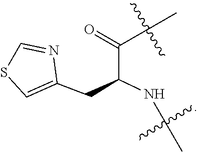 [SMILES: *N[C@@H](Cc1cscn1)C(*)=O]

In some embodiments, X⁵ is

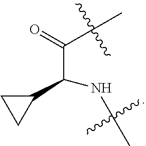 [SMILES: *N[C@H](C(*)=O)C1CC1]

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

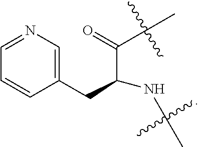 [SMILES: *N[C@@H](Cc1cccnc1)C(*)=O]

In some embodiments, X¹² is

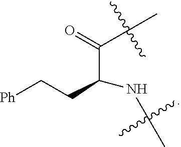 [SMILES: *N[C@@H](CCc1ccccc1)C(*)=O]

In some embodiments, each of X⁷, X¹⁰, and X¹¹ is independently an amino acid residue with a hydrophobic side chain (“hydrophobic amino acid residue”, Xaa^{H}). In some embodiments, X⁷ is Xaa^{H}. In some embodiments, X⁷ is

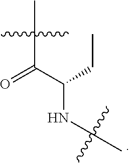 [SMILES: *N[C@@H](CC)C(*)=O]

In some embodiments, X⁷ is Val. In some embodiments, X¹⁰ is Xaa^{H}. In some embodiments, X¹⁰ is Met. In some embodiments, X¹⁰ is

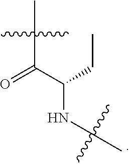 [SMILES: *N[C@@H](CC)C(*)=O]

In some embodiments, X¹¹ is Xaa^{H}. In some embodiments, X¹¹ is

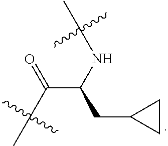 [SMILES: *N[C@@H](CC1CC1)C(*)=O]

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then an antibody binding moiety.

In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

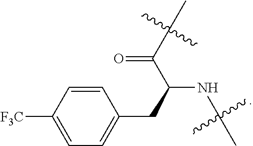 [SMILES: *N[C@@H](Cc1ccc(C(F)(F)F)cc1)C(*)=O]

In some embodiments, X¹² is

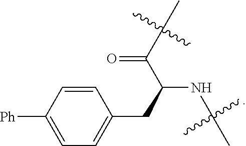 [SMILES: *N[C@@H](Cc1ccc(-c2ccccc2)cc1)C(*)=O]

In some embodiments, X¹² is

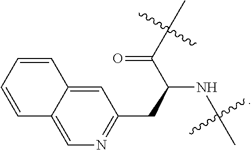 [SMILES: *N[C@@H](Cc1cc2ccccc2cn1)C(*)=O]

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

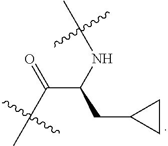 [SMILES: *N[C@@H](CC1CC1)C(*)=O]

In some embodiments, X¹¹ is Xaa^{H}. In some embodiments, X¹¹ is

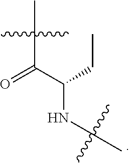 [SMILES: *N[C@@H](CC)C(*)=O]

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then an antibody binding moiety. In some embodiments, L^{b} is

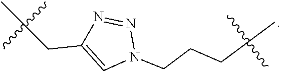 [SMILES: *CCCn1cc(C*)nn1]

In some embodiments, L^{b} is

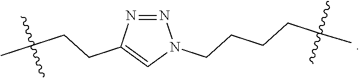 [SMILES: *CCCCn1cc(CC*)nn1]

In some embodiments, L^{b} connects two alpha-carbon atoms of two different amino acid residues. In some embodiments, both X⁵ and X¹⁰ are Cys, and the two —SH groups of their side chains form —S—S-(L^{b} is —CH₂—S—S—CH₂—).

In some embodiments, -(Xaa)z- is or comprises —X²X³X⁴X⁵X⁶X⁷X³X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹² are connected by L^{b}. In some embodiments, L^{b} is —CH₂—S—S—CH₂—. In some embodiments, L^{b} is —CH₂—CH₂—S—CH₂—. In some embodiments, L^{b} is

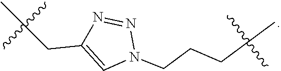 [SMILES: *CCCn1cc(C*)nn1]

In some embodiments, L^{b} is

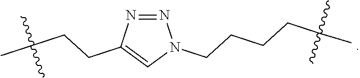 [SMILES: *CCCCn1cc(CC*)nn1]

In some embodiments, L^{b} is —CH₂CH₂CO—N(R′)—CH₂CH₂—. In some embodiments, R′ are taken together with an R group on the backbone atom that —N(R′)—CH₂CH₂— is bonded to form a ring, e.g., as in A-34. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

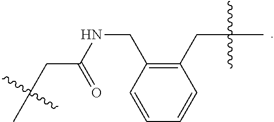 [SMILES: *CC(=O)NCc1ccccc1C*]

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X² are Cys, and the two —SH groups of their side chains form —S—S-(L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

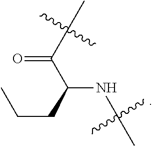 [SMILES: *N[C@@H](CCC)C(*)=O]

In some embodiments, X¹⁰ is Xaa^{H}. In some embodiments, X¹⁰ is Val. In some embodiments, X¹⁰ is

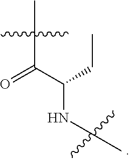 [SMILES: *N[C@@H](CC)C(*)=O]

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X¹³ is an amino acid residue comprising a polar uncharged side chain (e.g., at physiological pH, “polar uncharged amino acid residue”, Xaa^{L}). In some embodiments, X¹³ is Thr. In some embodiments, X¹³ is Val. In some embodiments, p13 is 0. In some embodiments, R^{c} is —NHCH₂CH(OH)CH₃. In some embodiments, R^{c} is (R)—NHCH₂CH(OH)CH₃. In some embodiments, R^{c} is (S)—NHCH₂CH(OH)CH₃.

In some embodiments, -(Xaa)z- is or comprises —X²X³X⁴X⁵X⁶X⁷X³X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

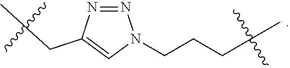 [SMILES: *CCCn1cc(C*)nn1]

In some embodiments, L^{b} is

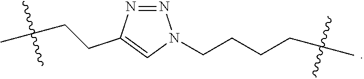 [SMILES: *CCCCn1cc(CC*)nn1]

In some embodiments, both X² and X¹² are Cys, and the two —SH groups of their side chains form —S—S-(L^{b} is —CH₂—S—S—CH₂—). In some embodiments, both X⁴ and X¹⁰ are Cys, and the two —SH groups of their side chains form —S—S-(L^{b} is —CH₂—S—S—CH₂—). In some embodiments, X⁴ and X⁹ are connected by L^{b}, wherein L^{b} is

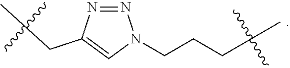 [SMILES: *CCCn1cc(C*)nn1]

In some embodiments, X⁴ and X⁹ are connected by L^{b}, wherein L^{b} is

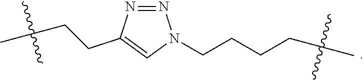 [SMILES: *CCCCn1cc(CC*)nn1]

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹¹ is Tyr. In some embodiments, X¹¹ is

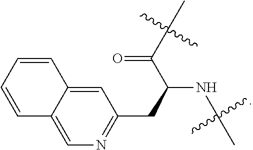 [SMILES: *N[C@@H](Cc1cc2ccccc2cn1)C(*)=O]

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

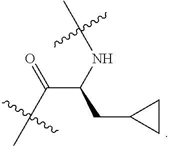 [SMILES: *N[C@@H](CC1CC1)C(*)=O]

In some embodiments, X⁹ is Xaa^{H}. In some embodiments, X⁹ is Leu. In some embodiments, X⁹ is

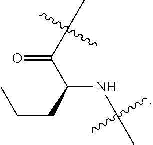 [SMILES: *N[C@@H](CCC)C(*)=O]

In some embodiments, X¹⁰ is Xaa^{H}. In some embodiments, X¹⁰ is Val. In some embodiments, X⁷ is Gly. In some embodiments, p1 is 1. In some embodiments, X¹ is Xaa^{N}. In some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Thr. In some embodiments, X¹³ is Val.

In some embodiments, -(Xaa)z- is or comprises —X²X³X⁴X⁵X⁶X⁷X⁸X⁹X¹⁰X¹¹X¹²X¹³X¹⁴X¹⁵X¹⁶—, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X¹³ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² are connected to X¹⁶ by L^{b}. In some embodiments, X⁴ are connected to X¹⁴ by L^{b}. In some embodiments, both X² and X¹⁶ are Cys, and the two —SH groups of their side chains form —S—S-(L^{b} is —CH₂—S—S—CH₂—). In some embodiments, both X⁴ and X¹⁴ are Cys, and the two —SH groups of their side chains form —S—S-(L^{b} is —CH₂—S—S—CH₂—). In some embodiments, L^{b} connects two alpha-carbon atoms of two different amino acid residues. In some embodiments, X³ is Asp. In some embodiments, X³ is Glu. In some embodiments, X⁵ is Xaa^{H}. In some embodiments, X⁵ is Ala. In some embodiments, X⁶ is Xaa^{A}. In some embodiments, X⁶ is Tyr. In some embodiments, X⁷ is Xaa^{A}. In some embodiments, X⁷ is Xaa^{P}. In some embodiments, X⁷ is His. In some embodiments, X⁸ is Xaa^{H}. In some embodiments, X⁸ is Ala. In some embodiments, X⁹ is Gly. In some embodiments, X¹⁰ is Asp. In some embodiments, X¹⁰ is Glu. In some embodiments, X¹¹ is Xaa^{H}. In some embodiments, X¹¹ is Leu. In some embodiments, X¹² is Xaa^{H}. In some embodiments, X¹² is Val. In some embodiments, X¹³ is Xaa^{A}. In some embodiments, X¹³ is Tyr. In some embodiments, X¹⁵ is Xaa^{L}. In some embodiments, X¹⁵ is Thr. In some embodiments, X¹⁵ is Val. In some embodiments, p1 is 1. In some embodiments, In some embodiments, X¹ is Xaa^{N}. In some embodiments, X¹ is Asp. In some embodiments, X¹ is Glu.

As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, lie, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In some embodiments, an antibody binding moiety is or comprises optionally substituted moiety of Table A-1. In some embodiments, a protein binding moiety is or comprises optionally substituted moiety of Table A-1. In some embodiments, an antibody binding moiety, e.g., a universal antibody binding moiety, is or comprises optionally substituted moiety of Table A-1. In some embodiments, an antibody binding moiety is selected from able A-1. In some embodiments, a protein binding moiety is selected from able A-1. In some embodiments, an antibody binding moiety, e.g., a universal antibody binding moiety, is selected from able A-1. In some embodiments, C-terminus and/or N-terminus are optionally capped (e.g., for C-terminus, by converting —COOH into —C(O)N(R′)₂ like —C(O)NH₂; for N-terminus, by adding R′C(O)— like CH₃C(O)— to an amino group).

TABLE A-1

| Exemplary antibody binding moieties. | |
|---|---|
| 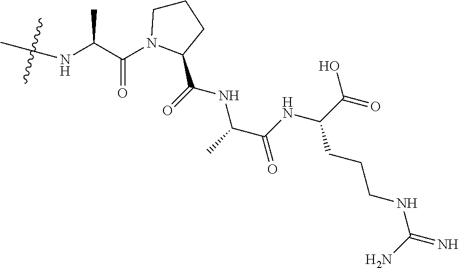 [SMILES: *N[C@@H](C)C(=O)N1CCC[C@H]1C(=O)N[C@@H](C)C(=O)N[C@@H](CCCNC(=N)N)C(=O)O] | A-1 |
| 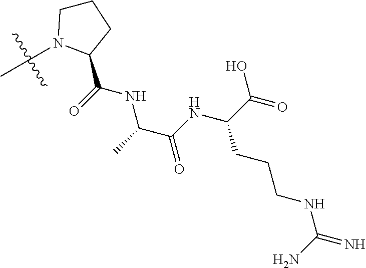 [SMILES: *N1CCC[C@H]1C(=O)N[C@@H](C)C(=O)N[C@@H](CCCNC(=N)N)C(=O)O] | A-2 |
| 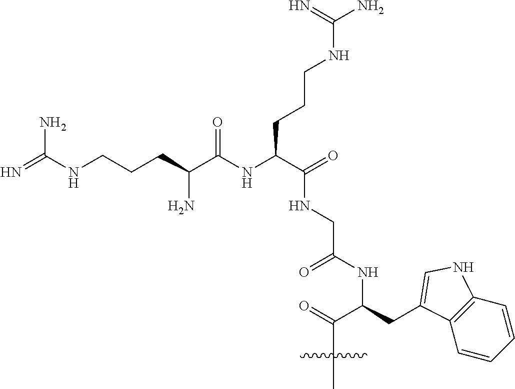 [SMILES: *C(=O)[C@H](Cc1c[nH]c2ccccc12)NC(=O)CNC(=O)[C@H](CCCNC(=N)N)NC(=O)[C@@H](N)CCCNC(=N)N] | A-3 |
| 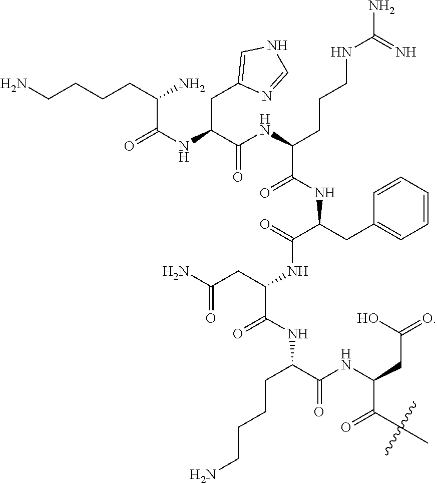 [SMILES: *C(=O)[C@H](CC(=O)O)NC(=O)[C@H](CCCCN)NC(=O)[C@H](CC(N)=O)NC(=O)[C@H](Cc1ccccc1)NC(=O)[C@H](CCCNC(=N)N)NC(=O)[C@H](Cc1c[nH]cn1)NC(=O)[C@@H](N)CCCCN] | A-4 |
| 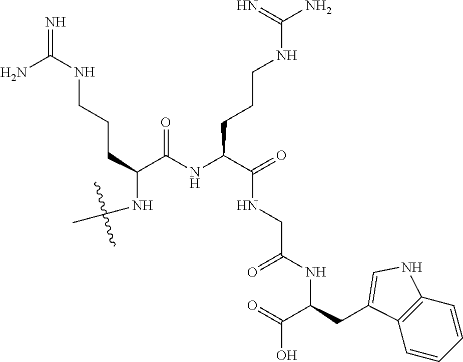 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O] | A-5 |
| 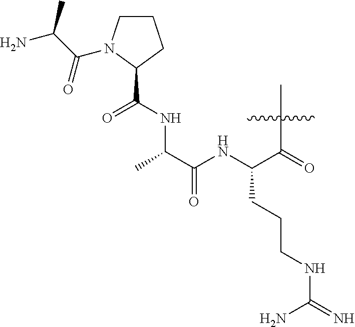 [SMILES: *C(=O)[C@H](CCCNC(=N)N)NC(=O)[C@H](C)NC(=O)[C@@H]1CCCN1C(=O)[C@H](C)N] | A-6 |
| 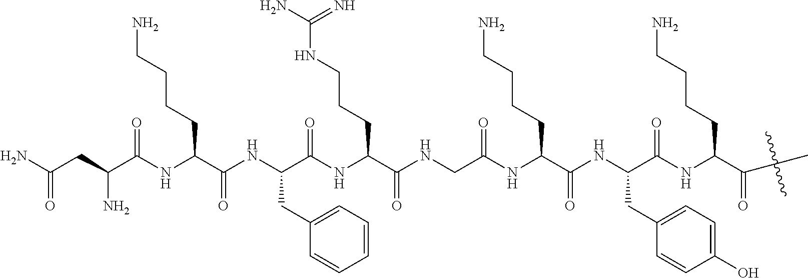 [SMILES: *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O] | A-7 |
| 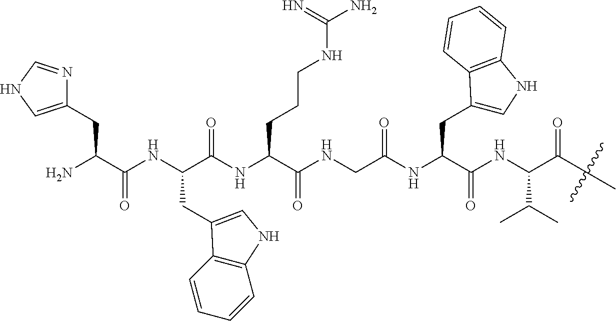 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C] | A-8 |
| 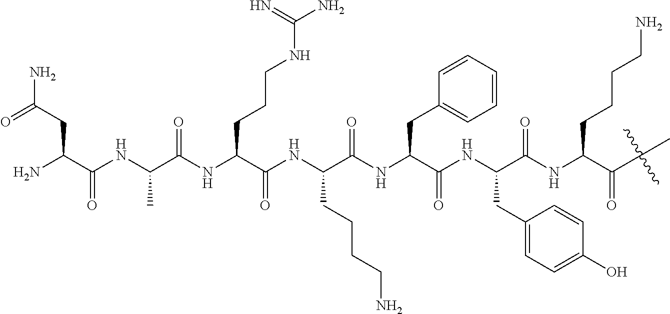 [SMILES: *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O] | A-9 |
| 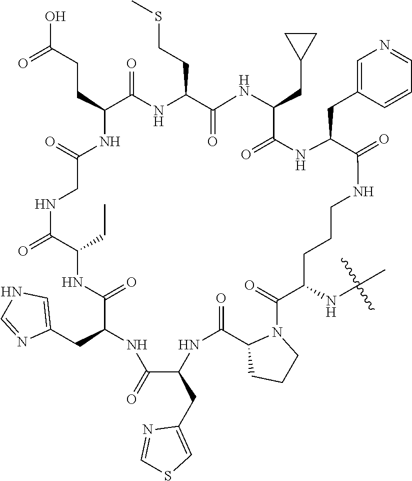 [SMILES: *N[C@H]1CCCNC(=O)[C@H](Cc2cccnc2)NC(=O)[C@H](CC2CC2)NC(=O)[C@H](CCSC)NC(=O)[C@H](CCC(=O)O)NC(=O)CNC(=O)[C@H](CC)NC(=O)[C@H](Cc2c[nH]cn2)NC(=O)[C@H](Cc2cscn2)NC(=O)[C@H]2CCCN2C1=O] | A-10 |
| 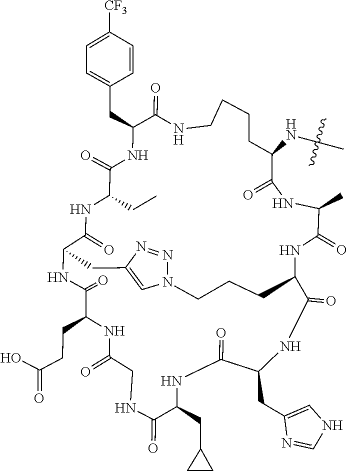 [SMILES: *N[C@@H]1CCCCNC(=O)[C@H](Cc2ccc(C(F)(F)F)cc2)NC(=O)[C@H](CC)NC(=O)[C@H]2Cc3cn(nn3)CCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-11 |
| 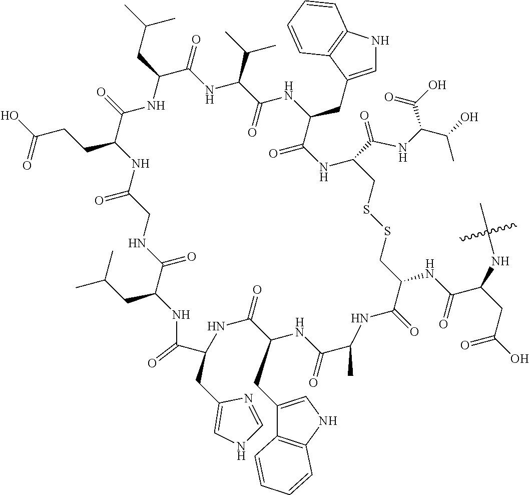 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-12 |
| 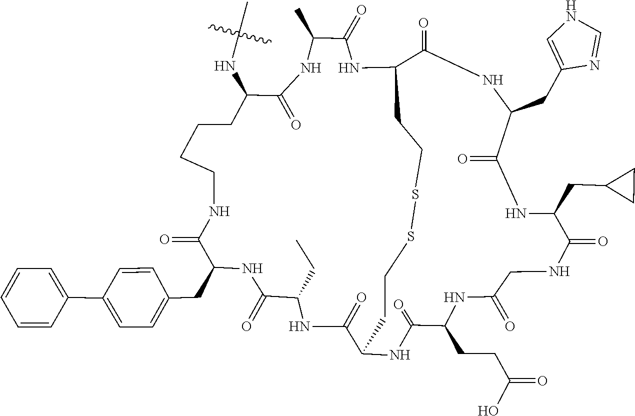 [SMILES: *N[C@@H]1CCCCNC(=O)[C@H](Cc2ccc(-c3ccccc3)cc2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-13 |
| 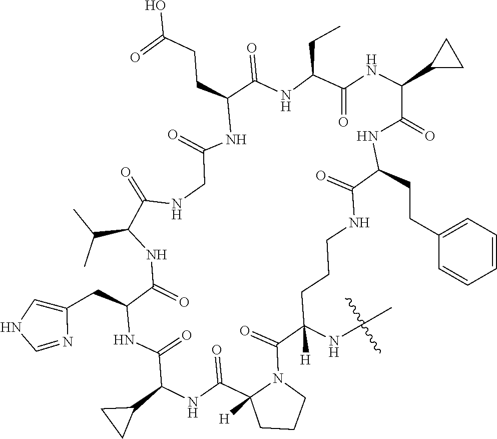 [SMILES: *N[C@H]1CCCNC(=O)[C@H](CCc2ccccc2)NC(=O)[C@H](C2CC2)NC(=O)[C@H](CC)NC(=O)[C@H](CCC(=O)O)NC(=O)CNC(=O)[C@H](C(C)C)NC(=O)[C@H](Cc2c[nH]cn2)NC(=O)[C@H](C2CC2)NC(=O)[C@H]2CCCN2C1=O] | A-14 |
| 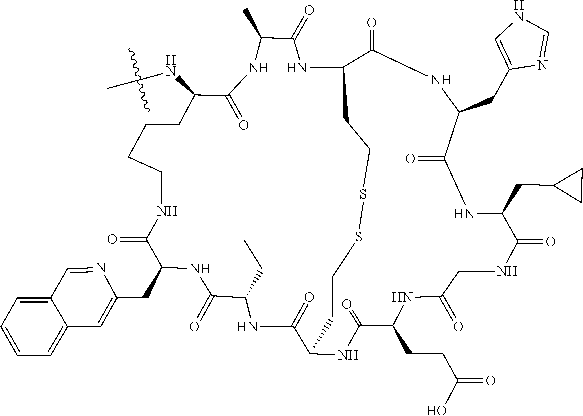 [SMILES: *N[C@@H]1CCCCNC(=O)[C@H](Cc2cc3ccccc3cn2)NC(=O)[C@H](CC)NC(=O)[C@H]2CCSSCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-15 |
| 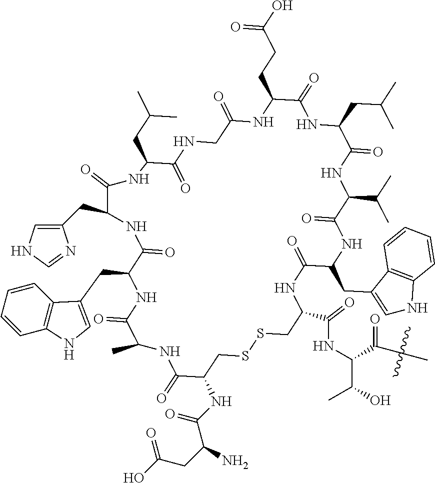 | A-16 |
| 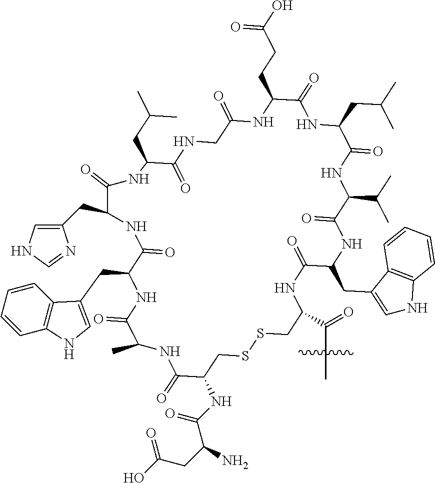 | A-17 |
| 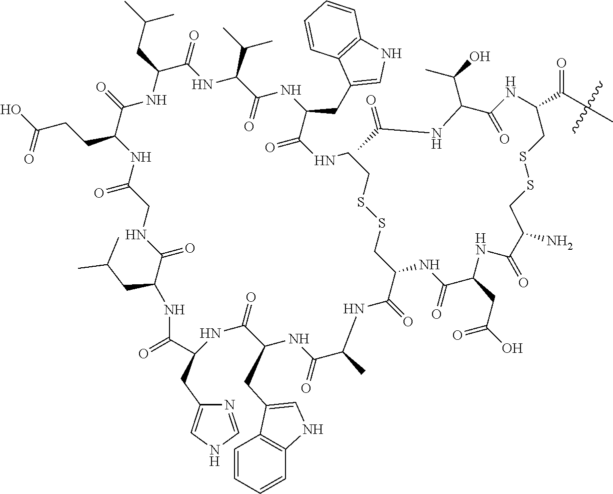 | A-18 |
| 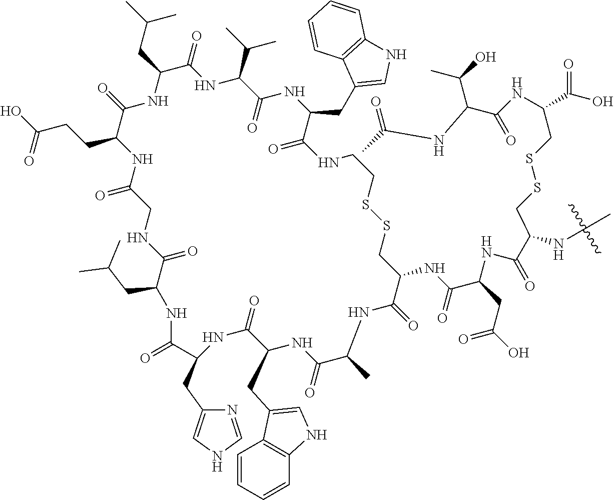 | A-19 |
| 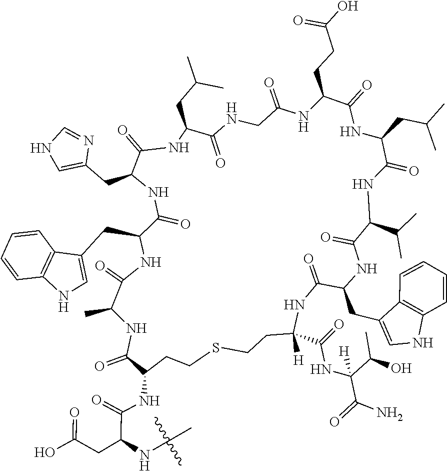 | A-20 |
| 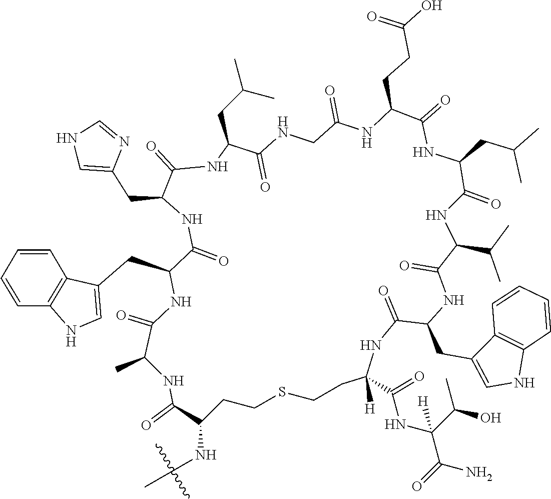 | A-21 |
| 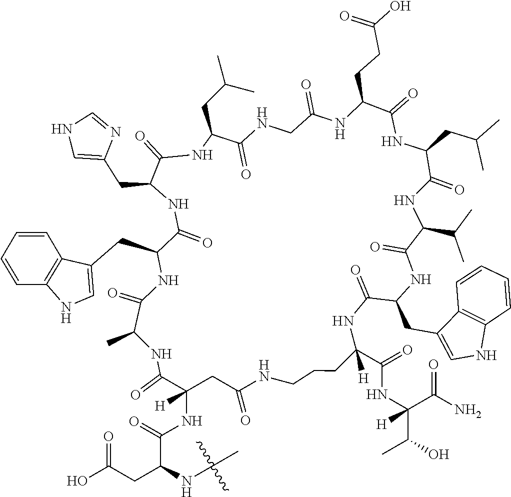 [SMILES: *N[C@@H](CC(=O)O)C(=O)N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-22 |
| 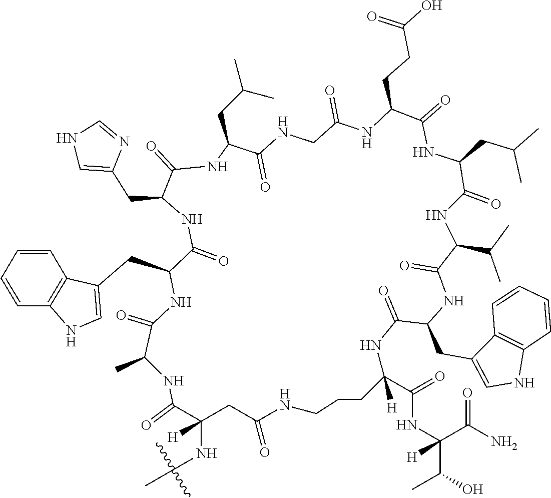 [SMILES: *N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-23 |
| 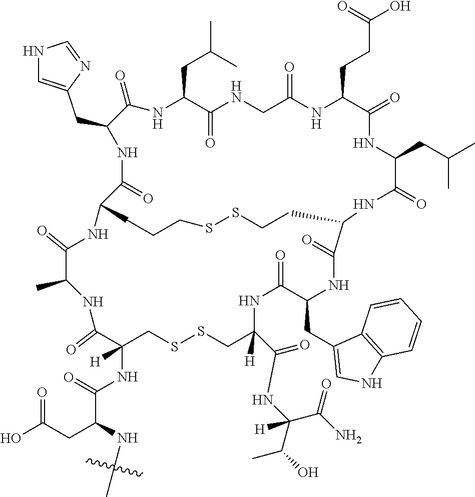 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-24 |
| 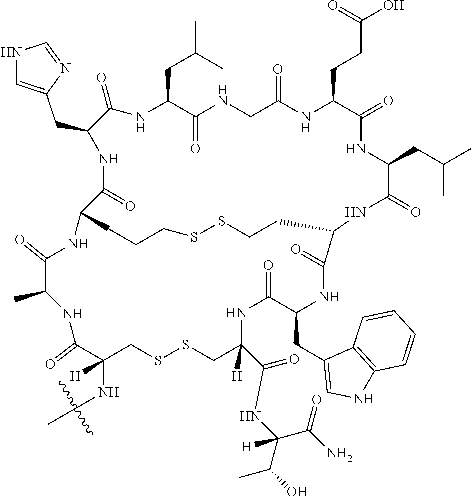 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-25 |
| 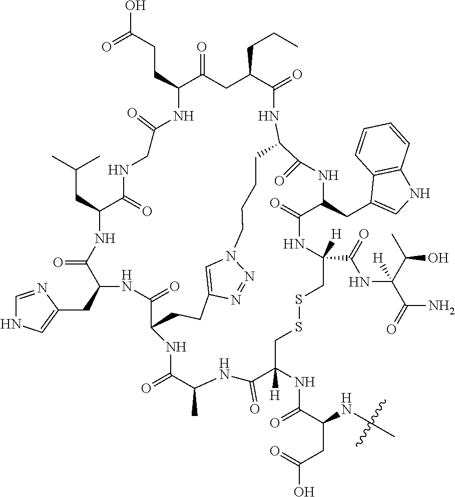 | A-26 |
| 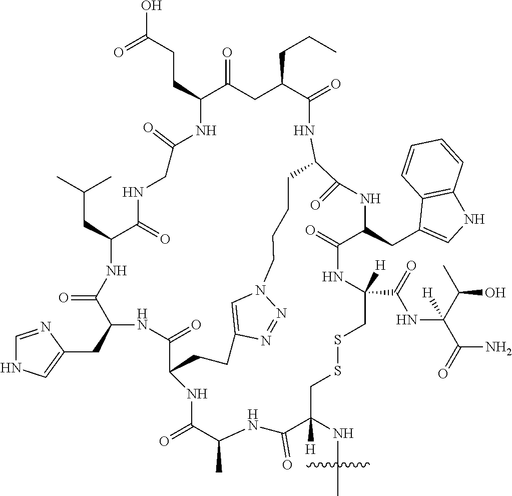 | A-27 |
| 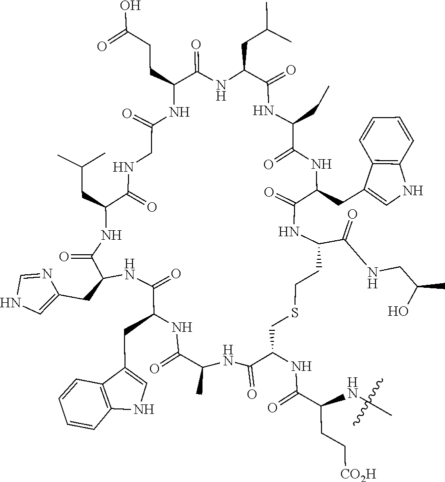 [SMILES: *N[C@@H](CCC(=O)O)C(=O)N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-28 |
| 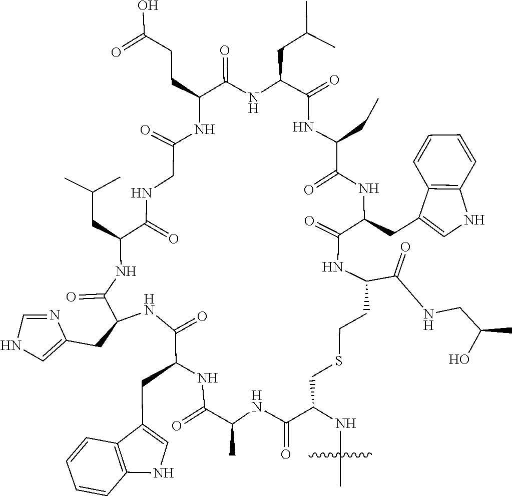 [SMILES: *N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-29 |
| 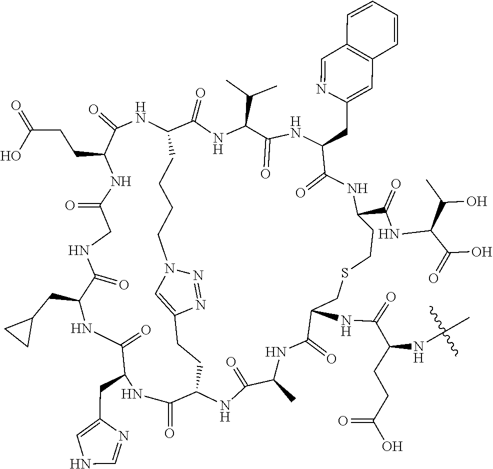 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-30 |
| 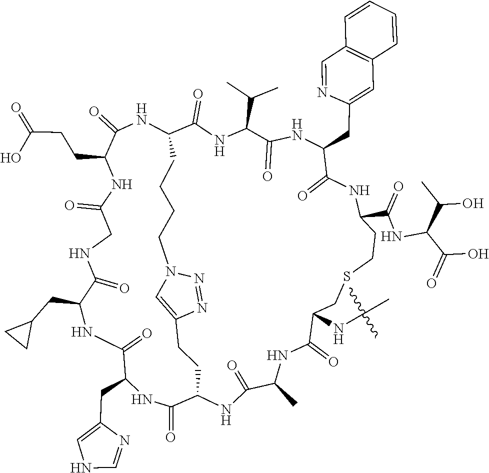 [SMILES: *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-31 |
| 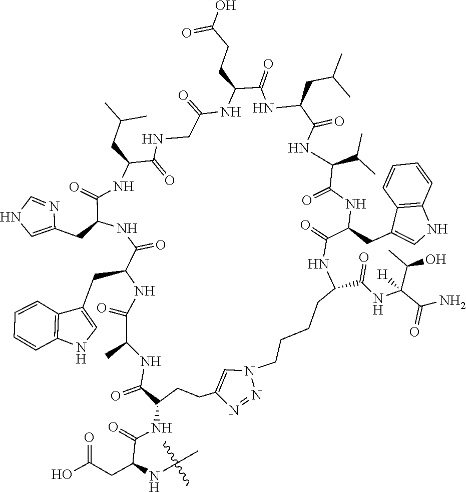 | A-32 |
| 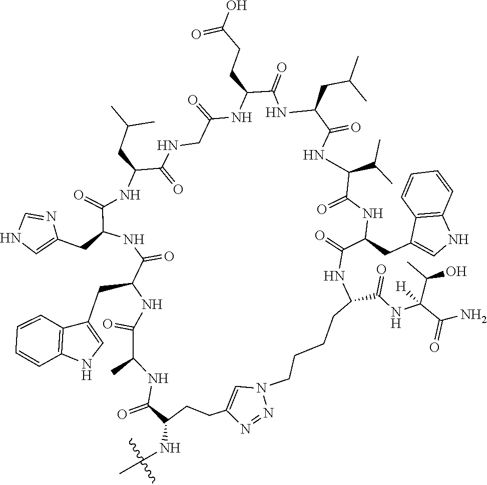 | A-33 |
| 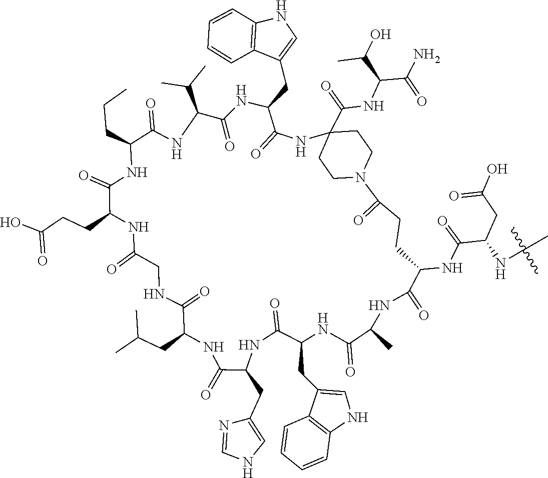 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-34 |
| 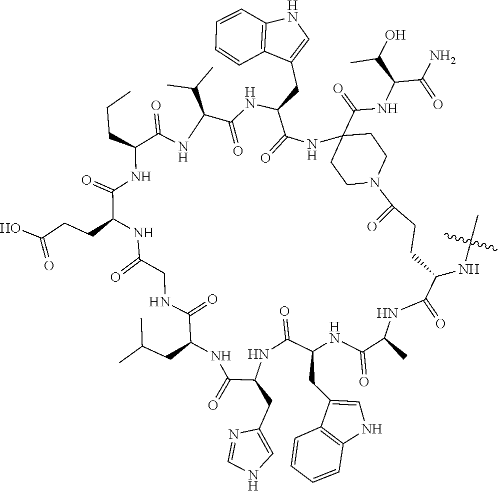 [SMILES: *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-35 |
| 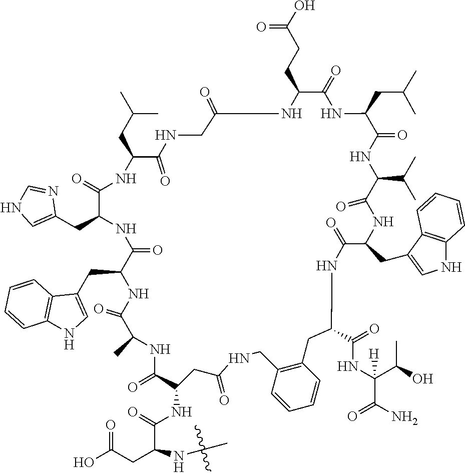 | A-36 |
| 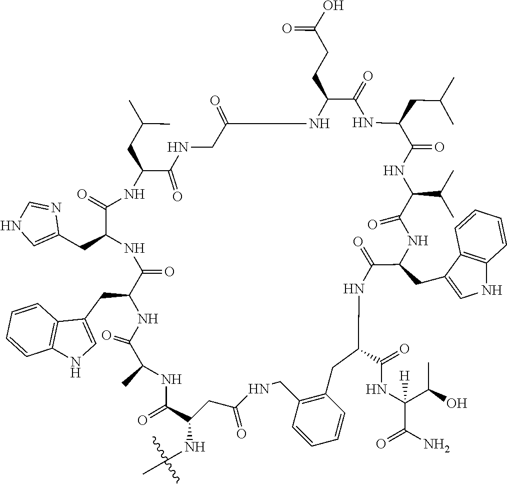 | A-37 |
| 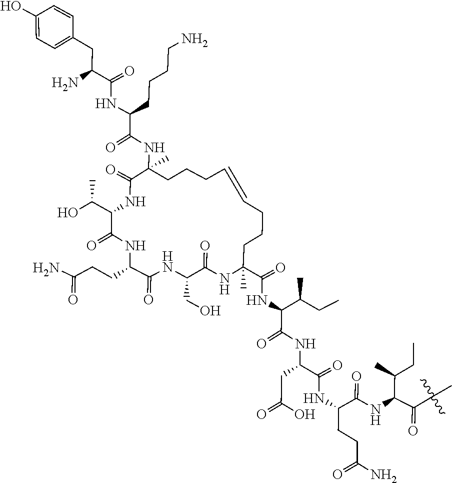 [SMILES: *C(=O)[C@@H](NC(=O)[C@H](CCC(N)=O)NC(=O)[C@H](CC(=O)O)NC(=O)[C@@H](NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1)[C@@H](C)CC)[C@@H](C)CC] | A-38 |
| 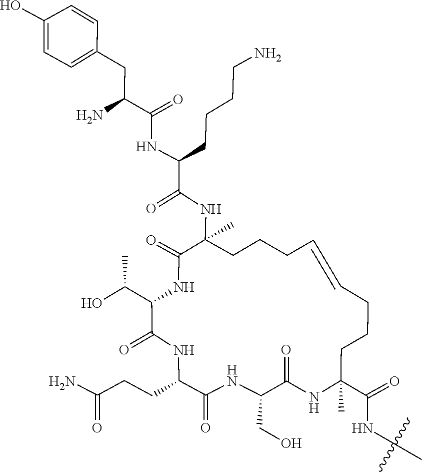 [SMILES: *NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1] | A-39 |
| 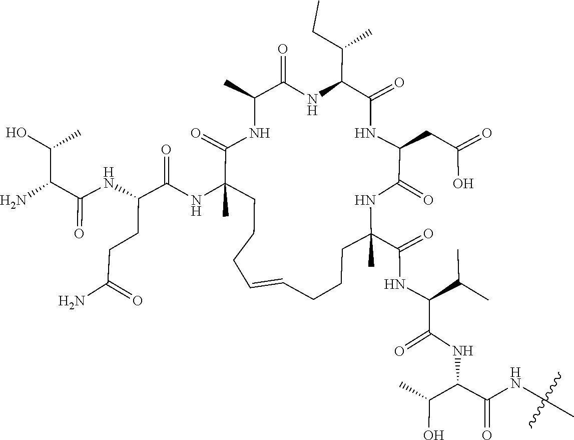 [SMILES: *NC(=O)[C@@H](NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C)[C@@H](C)O] | A-40 |
| 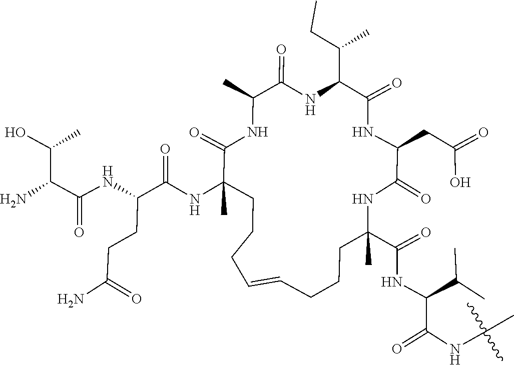 [SMILES: *NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C] | A-41 |
| 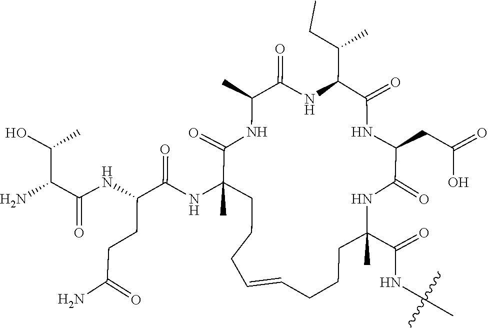 [SMILES: *NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1] | A-42 |
| 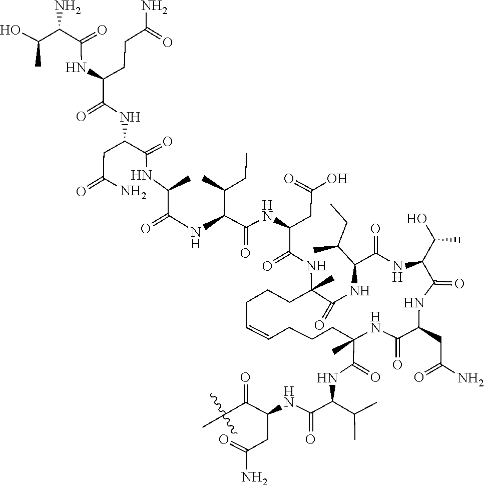 [SMILES: *C(=O)[C@H](CC(N)=O)NC(=O)[C@@H](NC(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1)C(C)C] | A-43 |
| 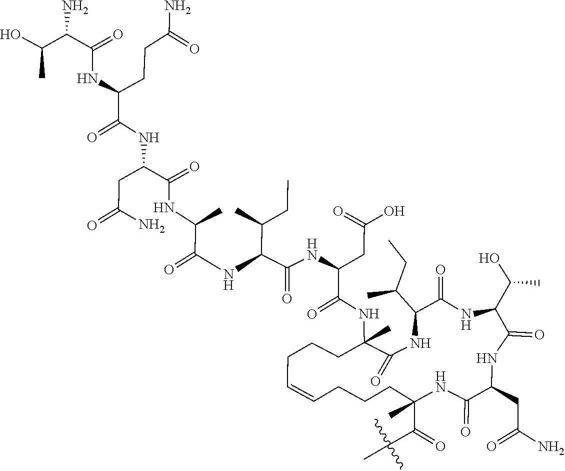 [SMILES: *C(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1] | A-44 |
| 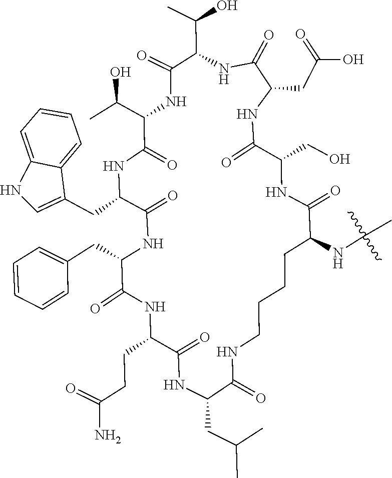 [SMILES: *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O] | A-45 |
| 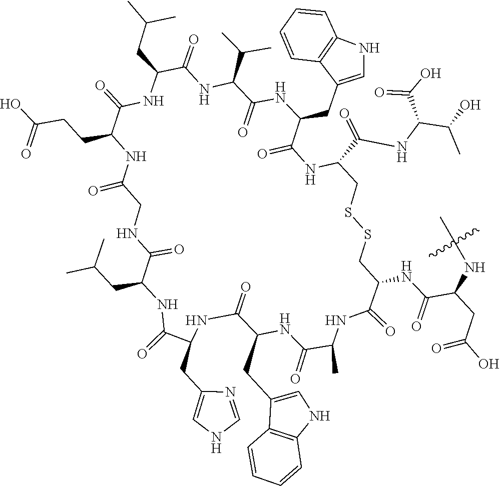 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-46 |
| 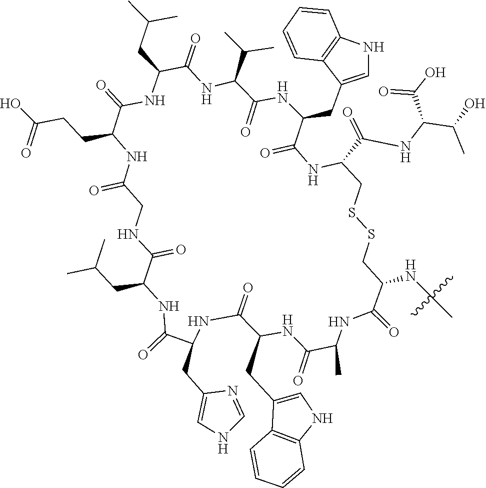 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-47 |
| 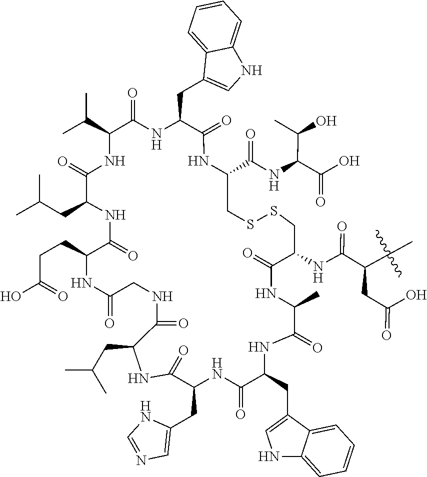 [SMILES: *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-48 |
| 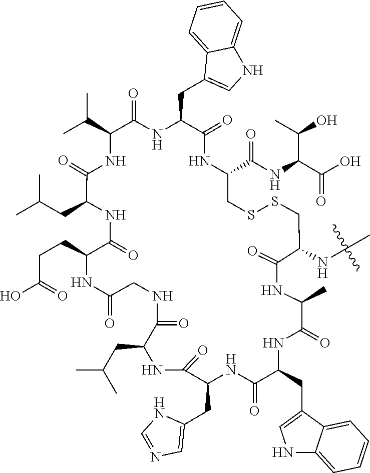 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-49 |
| 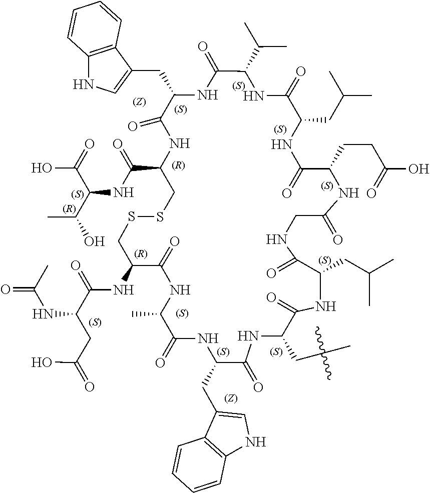 | A-50 |

In some embodiments, an antibody binding moiety is an antibody binding moiety described herein. In some embodiments, a protein binding moiety is an antibody binding moiety described herein. In some embodiments, —COOH and/or amino groups of amino acid residues, e.g., those at the C-terminus or N-terminus, is optionally capped. For example, in some embodiments, a —COOH group (e.g., a C-terminus —COOH) is amidated (e.g., converted into —CON(R′)₂, e.g., —C(O)NHR (e.g., —C(O)NH₂)), and in some embodiments, an amino group, e.g. —NH₂ (e.g., a N-terminus —NH₂) is capped with R′— or R′C(O)— (e.g., in some embodiments, by conversion —NH₂ into —NHR′ (e.g., —NHC(O)R, (e.g., —NHC(O)CH₃))).

In some embodiments, an antibody binding moiety is or comprises optionally substituted A-1. In some embodiments, an antibody binding moiety is or comprises optionally substituted A-2. In some embodiments, an antibody binding moiety is or comprises optionally substituted A-3. In some embodiments, an antibody binding moiety is or comprises optionally substituted A-4. In some embodiments, an antibody binding moiety is or comprises optionally substituted A-5. In some embodiments, an antibody binding moiety is or comprises optionally substituted A-6. In some embodiments, an antibody binding moiety is or comprises optionally substituted A-7. In some embodiments, an antibody binding moiety is or comprises optionally substituted A-8. In some embodiments, an antibody binding moiety is or comprises optionally substituted A-9. In some embodiments, an antibody binding moiety is or comprises optionally substituted A-10. In some embodiments, an antibody binding moiety is or comprises optionally substituted A-11. In some embodiments, an antibody binding moiety is or comprises optionally substituted A-12. In some embodiments, an antibody binding moiety is or comprises optionally substituted A-13. In some embodiments, an antibody binding moiety is or comprises optionally substituted A-14. In some embodiments, an antibody binding moiety is or comprises optionally substituted A-15. In some embodiments, an antibody binding moiety is or comprises optionally substituted A-16. In some embodiments, an antibody binding moiety is or comprises optionally substituted A-17. In some embodiments, an antibody binding moiety is or comprises optionally substituted A-18. In some embodiments, an antibody binding moiety is or comprises optionally substituted A-19. In some embodiments, an antibody binding moiety is or comprises optionally substituted A-20. In some embodiments, an antibody binding moiety is or comprises optionally substituted A-21. In some embodiments, an antibody binding moiety is or comprises optionally substituted A-22. In some embodiments, an antibody binding moiety is or comprises optionally substituted A-23. In some embodiments, an antibody binding moiety is or comprises optionally substituted A-24. In some embodiments, an antibody binding moiety is or comprises optionally substituted A-25. In some embodiments, an antibody binding moiety is or comprises optionally substituted A-26. In some embodiments, an antibody binding moiety is or comprises optionally substituted A-27. In some embodiments, an antibody binding moiety is or comprises optionally substituted A-28. In some embodiments, an antibody binding moiety is or comprises optionally substituted A-29. In some embodiments, an antibody binding moiety is or comprises optionally substituted A-30. In some embodiments, an antibody binding moiety is or comprises optionally substituted A-31. In some embodiments, an antibody binding moiety is or comprises optionally substituted A-32. In some embodiments, an antibody binding moiety is or comprises optionally substituted A-33. In some embodiments, an antibody binding moiety is or comprises optionally substituted A-34. In some embodiments, an antibody binding moiety is or comprises optionally substituted A-35. In some embodiments, an antibody binding moiety is or comprises optionally substituted A-36. In some embodiments, an antibody binding moiety is or comprises optionally substituted A-37. In some embodiments, an antibody binding moiety is or comprises optionally substituted A-38. In some embodiments, an antibody binding moiety is or comprises optionally substituted A-39. In some embodiments, an antibody binding moiety is or comprises optionally substituted A-40. In some embodiments, an antibody binding moiety is or comprises optionally substituted A-41. In some embodiments, an antibody binding moiety is or comprises optionally substituted A-42. In some embodiments, an antibody binding moiety is or comprises optionally substituted A-43. In some embodiments, an antibody binding moiety is or comprises optionally substituted A-44. In some embodiments, an antibody binding moiety is or comprises optionally substituted A-45. In some embodiments, an antibody binding moiety is or comprises optionally substituted A-46. In some embodiments, an antibody binding moiety is or comprises optionally substituted A-47. In some embodiments, an antibody binding moiety is or comprises optionally substituted A-48. In some embodiments, an antibody binding moiety is or comprises optionally substituted A-49. In some embodiments, an antibody binding moiety is or comprises optionally substituted A-50. In some embodiments, such an antibody binding moiety is an antibody binding moiety. In some embodiments, such an antibody binding moiety is a universal antibody binding moiety.

In some embodiments, an antibody binding moiety is A-1. In some embodiments, an antibody binding moiety is A-2. In some embodiments, an antibody binding moiety is A-3. In some embodiments, an antibody binding moiety is A-4. In some embodiments, an antibody binding moiety is A-5. In some embodiments, an antibody binding moiety is A-6. In some embodiments, an antibody binding moiety is A-7. In some embodiments, an antibody binding moiety is A-8. In some embodiments, an antibody binding moiety is A-9. In some embodiments, an antibody binding moiety is A-10. In some embodiments, an antibody binding moiety is A-11. In some embodiments, an antibody binding moiety is A-12. In some embodiments, an antibody binding moiety is A-13. In some embodiments, an antibody binding moiety is A-14. In some embodiments, an antibody binding moiety is A-15. In some embodiments, an antibody binding moiety is A-16. In some embodiments, an antibody binding moiety is A-17. In some embodiments, an antibody binding moiety is A-18. In some embodiments, an antibody binding moiety is A-19. In some embodiments, an antibody binding moiety is A-20. In some embodiments, an antibody binding moiety is A-21. In some embodiments, an antibody binding moiety is A-22. In some embodiments, an antibody binding moiety is A-23. In some embodiments, an antibody binding moiety is A-24. In some embodiments, an antibody binding moiety is A-25. In some embodiments, an antibody binding moiety is A-26. In some embodiments, an antibody binding moiety is A-27. In some embodiments, an antibody binding moiety is A-28. In some embodiments, an antibody binding moiety is A-29. In some embodiments, an antibody binding moiety is A-30. In some embodiments, an antibody binding moiety is A-31. In some embodiments, an antibody binding moiety is A-32. In some embodiments, an antibody binding moiety is A-33. In some embodiments, an antibody binding moiety is A-34. In some embodiments, an antibody binding moiety is A-35. In some embodiments, an antibody binding moiety is A-36. In some embodiments, an antibody binding moiety is A-37. In some embodiments, an antibody binding moiety is A-38. In some embodiments, an antibody binding moiety is A-39. In some embodiments, an antibody binding moiety is A-40. In some embodiments, an antibody binding moiety is A-41. In some embodiments, an antibody binding moiety is A-42. In some embodiments, an antibody binding moiety is A-43. In some embodiments, an antibody binding moiety is A-44. In some embodiments, an antibody binding moiety is A-45. In some embodiments, an antibody binding moiety is A-46. In some embodiments, an antibody binding moiety is A-47. In some embodiments, an antibody binding moiety is A-48. In some embodiments, an antibody binding moiety is A-49. In some embodiments, such an antibody binding moiety is an antibody binding moiety. In some embodiments, such an antibody binding moiety is a universal antibody binding moiety.

In some embodiments, an antibody binding moiety, e.g., a protein binding moiety (e.g., an antibody binding moiety (e.g., a universal antibody binding moiety)) comprises a peptide unit, and is connected to a linker moiety through the C-terminus of the peptide unit. In some embodiments, it is connected to a linker moiety through the N-terminus of the peptide unit. In some embodiments, it is connected to a linker through a side chain group of the peptide unit. In some embodiments, an antibody binding moiety, e.g., a universal antibody binding moiety comprises a peptide unit, and is connected to an antibody binding moiety optionally through a linker moiety through the C-terminus of the peptide unit. In some embodiments, an antibody binding moiety, e.g., a protein binding moiety (e.g., an antibody binding moiety (e.g., a universal antibody binding moiety)) comprises a peptide unit, and is connected to an antibody binding moiety optionally through a linker moiety through the N-terminus of the peptide unit. In some embodiments, In some embodiments, an antibody binding moiety, e.g., a protein binding moiety (e.g., an antibody binding moiety (e.g., a universal antibody binding moiety)) comprises a peptide unit, and is connected to an antibody binding moiety optionally through a linker moiety through a side chain of the peptide unit.

In some embodiments, an antibody binding moiety is or comprises (DCAWHLGELVWCT)—, wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, it is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues form a disulfide bond. In some embodiments, an antibody binding moiety is or comprises

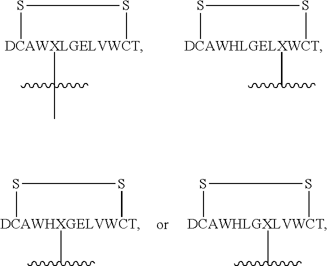

wherein X is an amino acid residue bonded to the rest of a compound or agent, and wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, the total number of replacements, deletions and insertions is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions. In some embodiments, there are no replacements. In some embodiments, an antibody binding moiety is or comprises

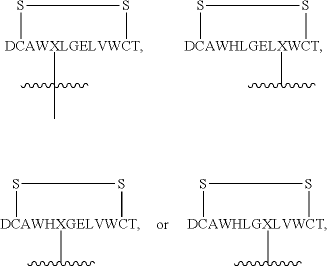

wherein X is an amino acid residue bonded to the rest of a compound or agent. In some embodiments, X is —N(R′)—CH(−)—C(O)—. In some embodiments, X is —N(R′)—CH(-L^{LG1}-)—C(O)—. In some embodiments, X is —N(R′)—CH(-L^{LG1}-L^{LG2}-)—C(O)—. In some embodiments, X is —N(R′)—CH(-L^{LG1}-L^{LG2}-L^{LG3}-)-C(O)—. In some embodiments, X is —N(R′)—CH(-LLG¹-L^{LG2}-L^{LG3}-L^{LG4}-)—C(O)—. In some embodiments, an antibody binding moiety is or comprises

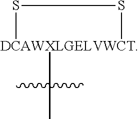

In some embodiments, an antibody binding moiety is or comprises

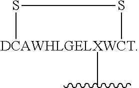

In some embodiments, an antibody binding moiety is or comprises

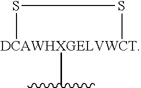

In some embodiments, an antibody binding moiety is or comprises

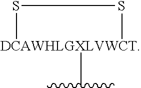

In some embodiments, X is a residue of

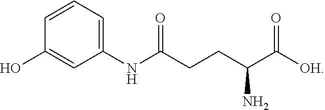 [SMILES: N[C@@H](CCC(=O)Nc1cccc(O)c1)C(=O)O]

In some embodiments, X is a residue of

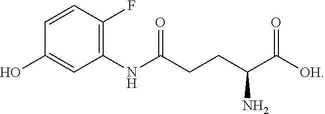 [SMILES: N[C@@H](CCC(=O)Nc1cc(O)ccc1F)C(=O)O]

In some embodiments, X is a residue of

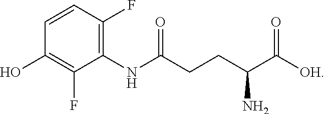 [SMILES: N[C@@H](CCC(=O)Nc1c(F)ccc(O)c1F)C(=O)O]

In some embodiments, X is a residue of

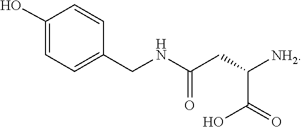 [SMILES: N[C@@H](CC(=O)NCc1ccc(O)cc1)C(=O)O]

In some embodiments, X is a residue of

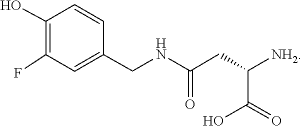 [SMILES: N[C@@H](CC(=O)NCc1ccc(O)c(F)c1)C(=O)O]

In some embodiments, X is a residue of

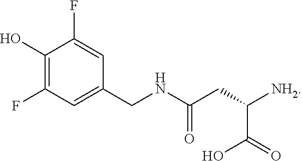 [SMILES: N[C@@H](CC(=O)NCc1cc(F)c(O)c(F)c1)C(=O)O]

In some embodiments, X is a residue of

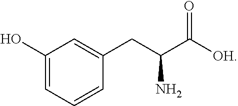 [SMILES: N[C@@H](Cc1cccc(O)c1)C(=O)O]

In some embodiments, X is a residue of

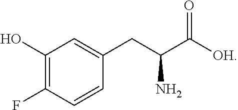 [SMILES: N[C@@H](Cc1ccc(F)c(O)c1)C(=O)O]

In some embodiments, X is a residue of

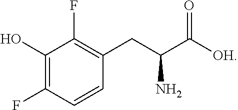 [SMILES: N[C@@H](Cc1ccc(F)c(O)c1F)C(=O)O]

In some embodiments, X is K. In some embodiments, X is D. In some embodiments, X is a residue of Dab. In some embodiments, X is E. In some embodiments, X is a residue of

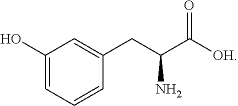 [SMILES: N[C@@H](Cc1cccc(O)c1)C(=O)O]

In some embodiments, the present disclosure provides an amino acid having the structure of

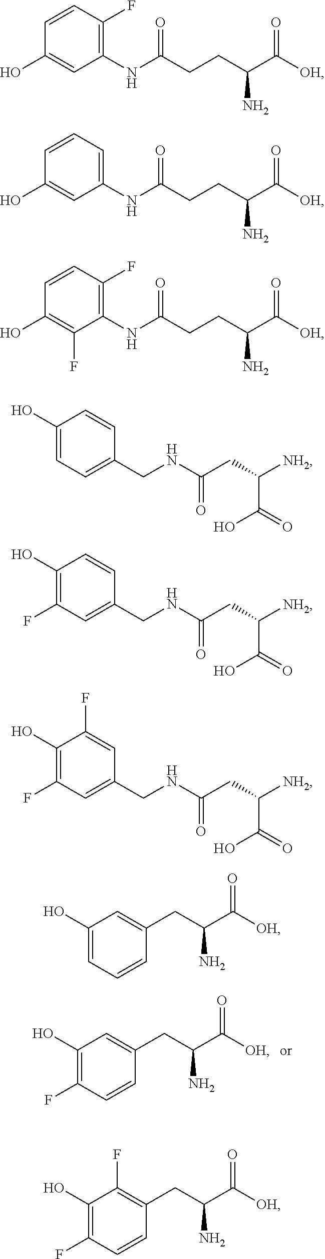

or a salt thereof, or an ester thereof, or an activated ester thereof, or a stereoisomer thereof, or an ester or an activated ester of a stereoisomer. In some embodiments, such antibody binding moieties are antibody binding moieties.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in U.S. Pat. No. 9,745,339, US 201/30131321, etc. In some embodiments, an antibody binding moiety is of such a structure that its corresponding compound is a compound described in U.S. Pat. No. 9,745,339 or US 2013/0131321, the compounds of each of which are independently incorporated herein by reference. In some embodiments, an antibody binding moiety ABT is of such a structure that H-ABT is a compound described in U.S. Pat. No. 9,745,339 or US 2013/0131321, the compounds of each of which are independently incorporated herein by reference. In some embodiments, such a compound can bind to an antibody. In some embodiments, such a compound can bind to Fc region of an antibody.

In some embodiments, an antibody binding moiety is or comprises optionally substituted

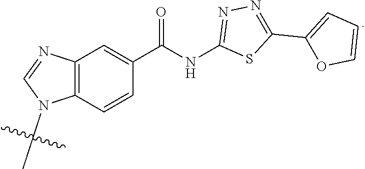 [SMILES: *n1cnc2cc(C(=O)Nc3nnc(-c4ccco4)s3)ccc21]

In some embodiments, an antibody binding moiety is or comprises

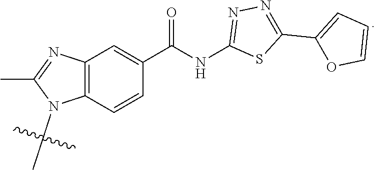 [SMILES: *n1c(C)nc2cc(C(=O)Nc3nnc(-c4ccco4)s3)ccc21]

In some embodiments, an antibody binding moiety is or comprises optionally substituted

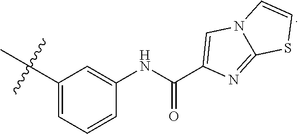 [SMILES: *c1cccc(NC(=O)c2cn3ccsc3n2)c1]

In some embodiments, an antibody binding moiety is or comprises

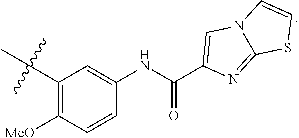 [SMILES: *c1cc(NC(=O)c2cn3ccsc3n2)ccc1OC]

In some embodiments, an antibody binding moiety is or comprises

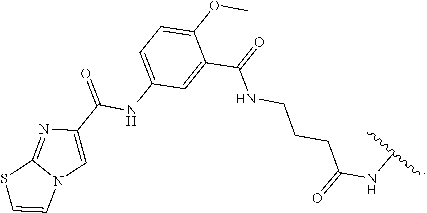 [SMILES: *NC(=O)CCCNC(=O)c1cc(NC(=O)c2cn3ccsc3n2)ccc1OC]

In some embodiments, an antibody binding moiety is or comprises optionally substituted

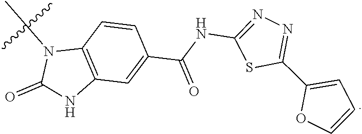 [SMILES: *n1c(=O)[nH]c2cc(C(=O)Nc3nnc(-c4ccco4)s3)ccc21]

In some embodiments, an antibody binding moiety is or comprises

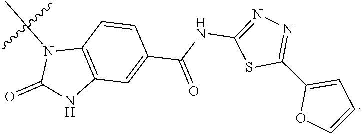 [SMILES: *n1c(=O)[nH]c2cc(C(=O)Nc3nnc(-c4ccco4)s3)ccc21]

In some embodiments, an antibody binding moiety is or comprises optionally substituted

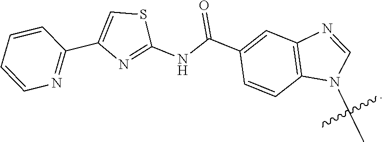 [SMILES: *n1cnc2cc(C(=O)Nc3nc(-c4ccccn4)cs3)ccc21]

In some embodiments, an antibody binding moiety is or comprises

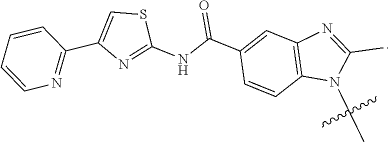 [SMILES: *n1c(C)nc2cc(C(=O)Nc3nc(-c4ccccn4)cs3)ccc21]

In some embodiments, antibody binding moiety is or comprises

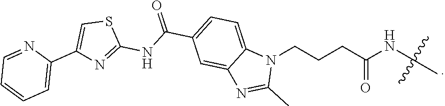 [SMILES: *NC(=O)CCCn1c(C)nc2cc(C(=O)Nc3nc(-c4ccccn4)cs3)ccc21]

In some embodiments, an antibody binding moiety is or comprises

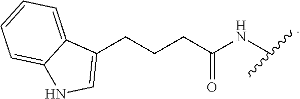 [SMILES: *NC(=O)CCCc1c[nH]c2ccccc12]

In some embodiments, antibody binding moiety is or comprises optionally substituted

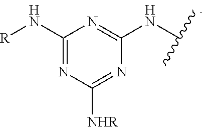

In some embodiments, antibody binding moiety is or comprises optionally substituted

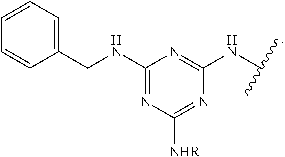

In some embodiments, antibody binding moiety is or comprises

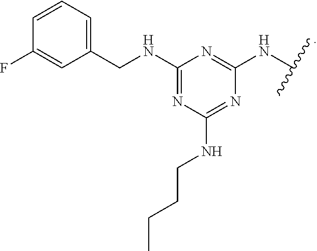 [SMILES: *Nc1nc(NCCCC)nc(NCc2cccc(F)c2)n1]

In some embodiments, an antibody binding moiety is or comprises optionally substituted

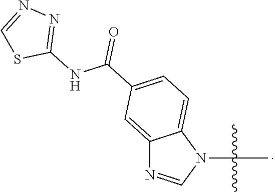 [SMILES: *n1cnc2cc(C(=O)Nc3nncs3)ccc21]

In some embodiments, an antibody binding moiety is or comprises

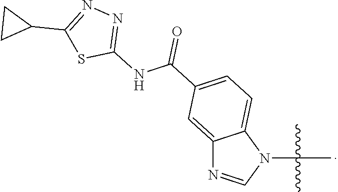 [SMILES: *n1cnc2cc(C(=O)Nc3nnc(C4CC4)s3)ccc21]

In some embodiments, an antibody binding moiety is or comprises

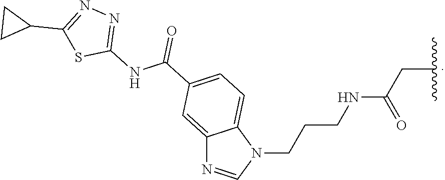 [SMILES: *CC(=O)NCCCn1cnc2cc(C(=O)Nc3nnc(C4CC4)s3)ccc21]

In some embodiments, an antibody binding moiety is or comprises optionally substituted

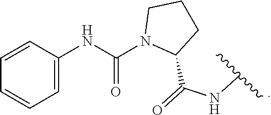 [SMILES: *NC(=O)[C@H]1CCCN1C(=O)Nc1ccccc1]

In some embodiments, an antibody binding moiety is or comprises

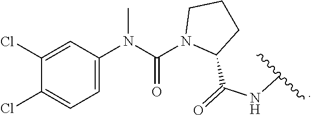 [SMILES: *NC(=O)[C@H]1CCCN1C(=O)N(C)c1ccc(Cl)c(Cl)c1]

In some embodiments, an antibody binding moiety is or comprises optionally substituted

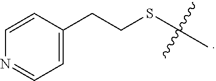 [SMILES: *SCCc1ccncc1]

In some embodiments, an antibody binding moiety is or comprises

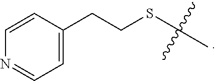 [SMILES: *SCCc1ccncc1]

In some embodiments, such antibody binding moieties are antibody binding moieties.

In some embodiments, antibody binding moiety is or comprises

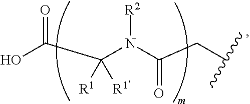

wherein each variable is independently as described herein. In some embodiments, m is 4 to 13. In some embodiments, an antibody binding moiety is or comprises

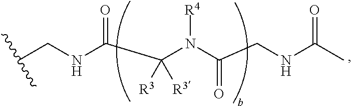

wherein b is 1-20, and each other variable is independently as described herein. In some embodiments, b is 4-13. In some embodiments, an antibody binding moiety, e.g., R^{c}-(Xaa)z-, is or comprises

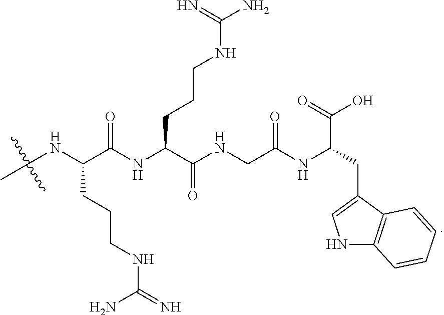 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O]

In some embodiments, an antibody binding moiety, e.g., R^{c}-(Xaa)z-, is or comprises

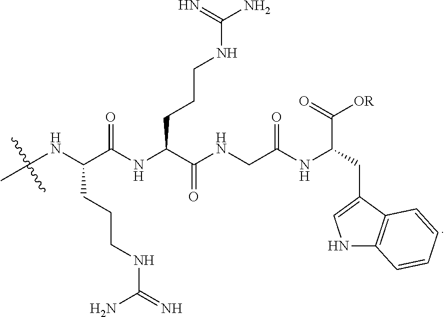

In some embodiments, an antibody binding moiety, e.g., R^{c}-(Xaa)z-, is or comprises

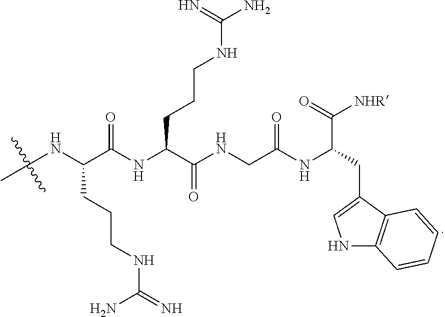

In some embodiments, an antibody binding moiety, e.g., R^{c}-(Xaa)z-, is or comprises

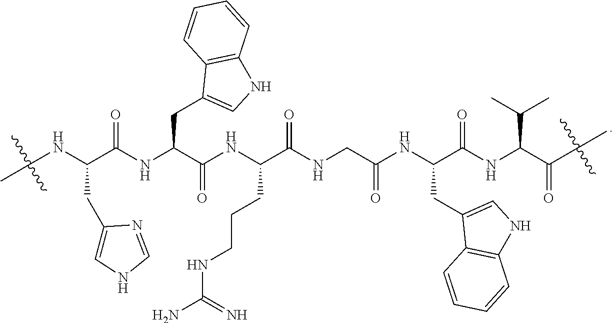 [SMILES: *N[C@@H](Cc1c[nH]cn1)C(=O)N[C@@H](Cc1c[nH]c2ccccc12)C(=O)N[C@@H](CCCNC(=N)N)C(=O)NCC(=O)N[C@@H](Cc1c[nH]c2ccccc12)C(=O)N[C@H](C(*)=O)C(C)C]

In some embodiments, an antibody binding moiety, e.g., R^{c}-(Xaa)z-, is or comprises

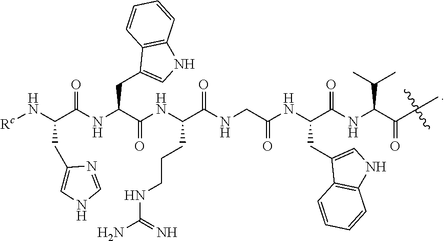

In some embodiments, an antibody binding moiety, e.g., R^{c}-(Xaa)z-, is or comprises

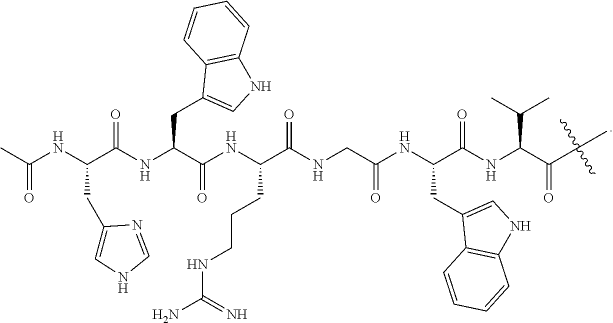 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@H](Cc1c[nH]cn1)NC(C)=O)C(C)C]

In some embodiments, an antibody binding moiety, e.g., R^{c}-(Xaa)z-, is or comprises

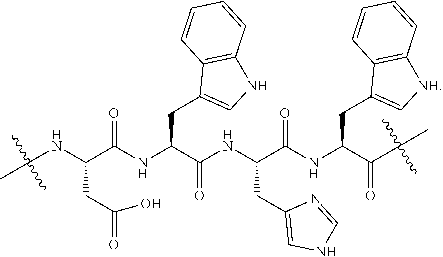 [SMILES: *N[C@@H](CC(=O)O)C(=O)N[C@@H](Cc1c[nH]c2ccccc12)C(=O)N[C@@H](Cc1c[nH]cn1)C(=O)N[C@@H](Cc1c[nH]c2ccccc12)C(*)=O]

In some embodiments, an antibody binding moiety, e.g., R^{c}-(Xaa)z-, is or comprises

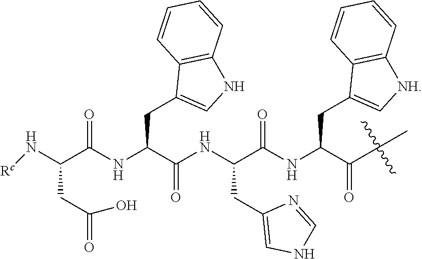

In some embodiments, an antibody binding moiety, e.g., R^{c}-(Xaa)z-, is or comprises

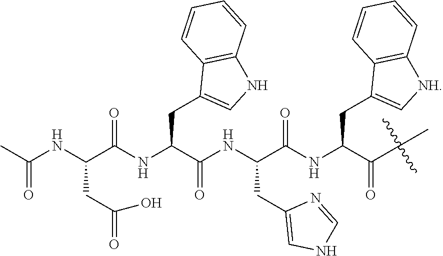 [SMILES: *C(=O)[C@H](Cc1c[nH]c2ccccc12)NC(=O)[C@H](Cc1c[nH]cn1)NC(=O)[C@H](Cc1c[nH]c2ccccc12)NC(=O)[C@H](CC(=O)O)NC(C)=O]

In some embodiments, an antibody binding moiety, e.g., R^{c}-(Xaa)z-, is or comprises

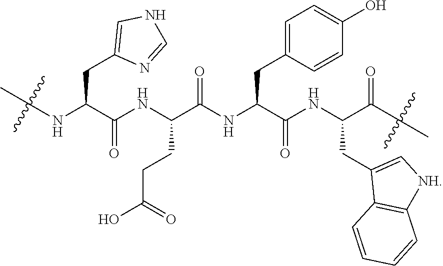 [SMILES: *N[C@@H](Cc1c[nH]cn1)C(=O)N[C@@H](CCC(=O)O)C(=O)N[C@@H](Cc1ccc(O)cc1)C(=O)N[C@@H](Cc1c[nH]c2ccccc12)C(*)=O]

In some embodiments, an antibody binding moiety, e.g., R^{c}-(Xaa)z-, is or comprises

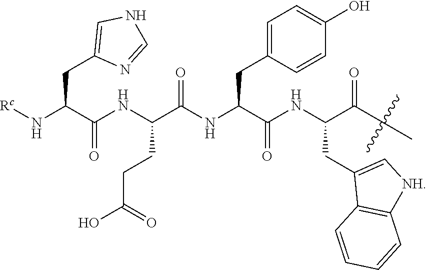

In some embodiments, an antibody binding moiety, e.g., R^{c}-(Xaa)z-, is or comprises

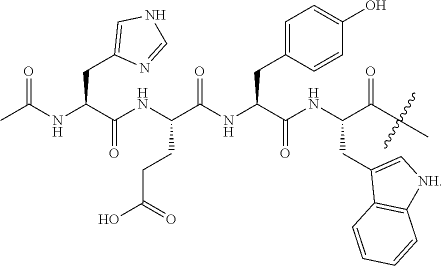 [SMILES: *C(=O)[C@H](Cc1c[nH]c2ccccc12)NC(=O)[C@H](Cc1ccc(O)cc1)NC(=O)[C@H](CCC(=O)O)NC(=O)[C@H](Cc1c[nH]cn1)NC(C)=O]

In some embodiments, an antibody binding moiety, e.g., R^{c}-(Xaa)z-, is or comprises

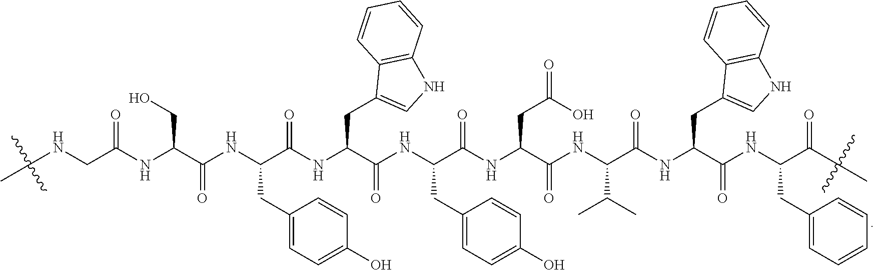 [SMILES: *NCC(=O)N[C@@H](CO)C(=O)N[C@@H](Cc1ccc(O)cc1)C(=O)N[C@@H](Cc1c[nH]c2ccccc12)C(=O)N[C@@H](Cc1ccc(O)cc1)C(=O)N[C@@H](CC(=O)O)C(=O)N[C@H](C(=O)N[C@@H](Cc1c[nH]c2ccccc12)C(=O)N[C@@H](Cc1ccccc1)C(*)=O)C(C)C]

In some embodiments, an antibody binding moiety, e.g., R^{c}-(Xaa)z-, is or comprises

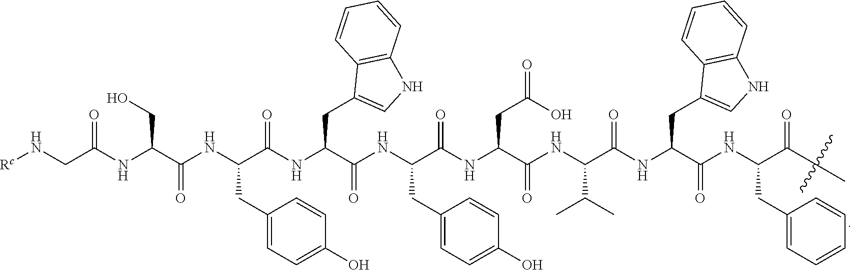

In some embodiments, an antibody binding moiety, e.g., R^{c}-(Xaa)z-, is or comprises

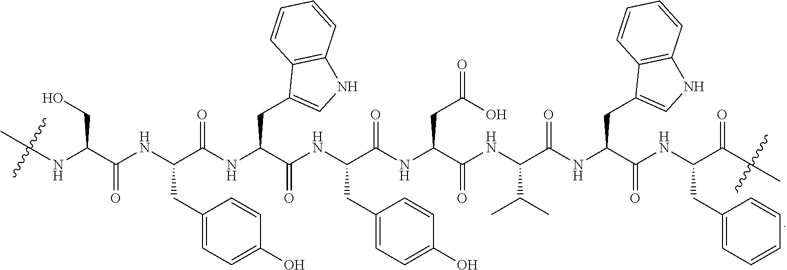 [SMILES: *N[C@@H](CO)C(=O)N[C@@H](Cc1ccc(O)cc1)C(=O)N[C@@H](Cc1c[nH]c2ccccc12)C(=O)N[C@@H](Cc1ccc(O)cc1)C(=O)N[C@@H](CC(=O)O)C(=O)N[C@H](C(=O)N[C@@H](Cc1c[nH]c2ccccc12)C(=O)N[C@@H](Cc1ccccc1)C(*)=O)C(C)C]

In some embodiments, an antibody binding moiety, e.g., R^{c}-(Xaa)z-, is or comprises

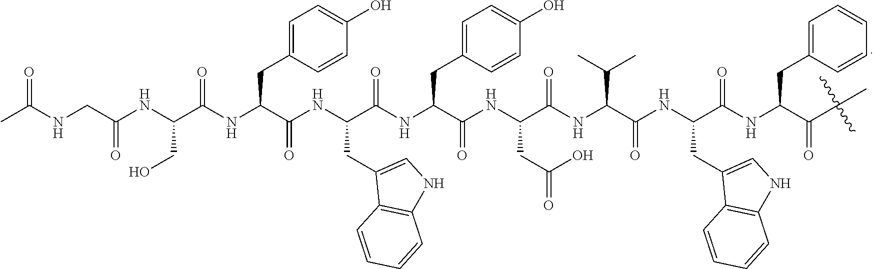 [SMILES: *C(=O)[C@H](Cc1ccccc1)NC(=O)[C@H](Cc1c[nH]c2ccccc12)NC(=O)[C@@H](NC(=O)[C@H](CC(=O)O)NC(=O)[C@H](Cc1ccc(O)cc1)NC(=O)[C@H](Cc1c[nH]c2ccccc12)NC(=O)[C@H](Cc1ccc(O)cc1)NC(=O)[C@H](CO)NC(=O)CNC(C)=O)C(C)C]

In some embodiments, an antibody binding moiety, e.g., R^{c}-(Xaa)z-, is or comprises

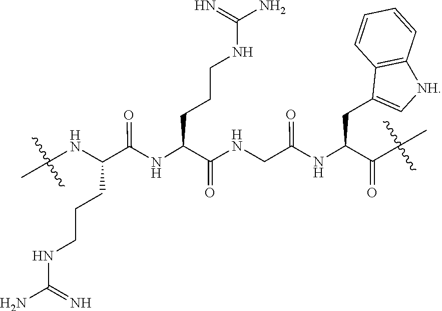 [SMILES: *N[C@@H](CCCNC(=N)N)C(=O)N[C@@H](CCCNC(=N)N)C(=O)NCC(=O)N[C@@H](Cc1c[nH]c2ccccc12)C(*)=O]

In some embodiments, an antibody binding moiety, e.g., R^{c}-(Xaa)z-, is or comprises

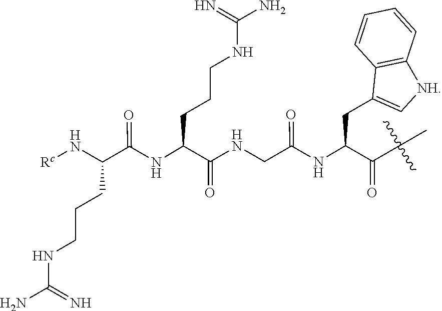

In some embodiments, an antibody binding moiety, e.g., R^{c}-(Xaa)z-, is or comprises

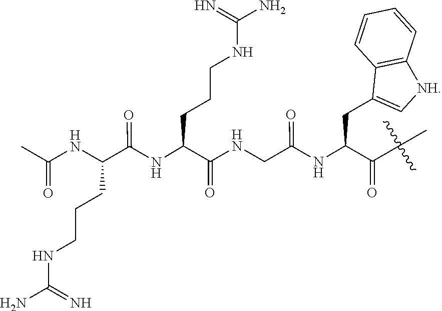 [SMILES: *C(=O)[C@H](Cc1c[nH]c2ccccc12)NC(=O)CNC(=O)[C@H](CCCNC(=N)N)NC(=O)[C@H](CCCNC(=N)N)NC(C)=O]

In some embodiments, —NH— is bonded to a R^{c} group. In some embodiments, R^{c} is R—C(O)—. In some embodiments, R^{c} is CH₃C(O)—. In some embodiments, such antibody binding moieties are antibody binding moieties.

In some embodiments, an antibody binding moiety, e.g.,

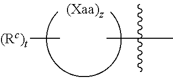

or R^{c}-(Xaa)z-, is or comprises

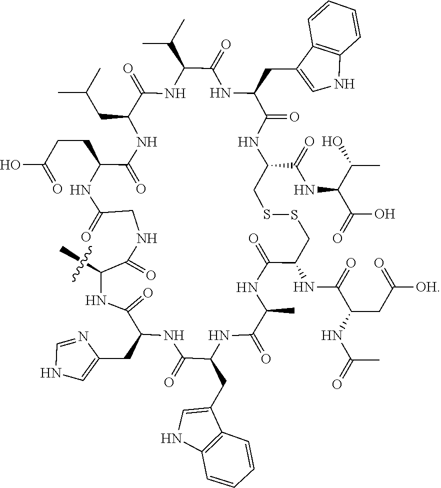

In some embodiments, an antibody binding moiety, e.g.,

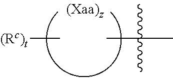

or R^{c}-(Xaa)z-, is or comprises

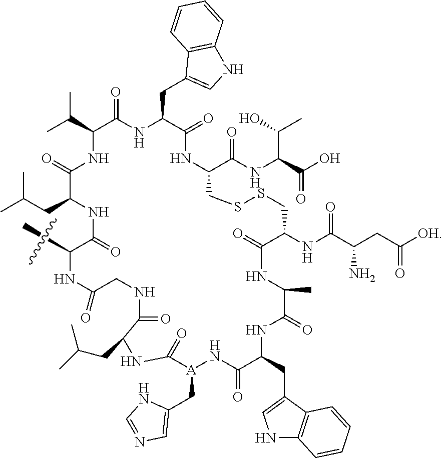

In some embodiments, an antibody binding moiety, e.g.,

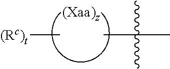

or R^{c}-(Xaa)z-, is or comprises

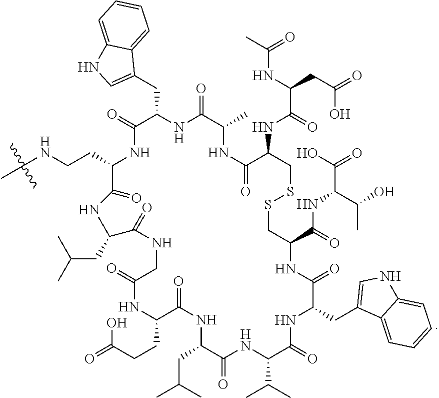 [SMILES: *NCC[C@@H]1NC(=O)[C@H](Cc2c[nH]c3ccccc23)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC1=O ; "."]

In some embodiments, an antibody binding moiety, e.g.,

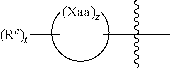

or R^{c}-(Xaa)z-, is or comprises

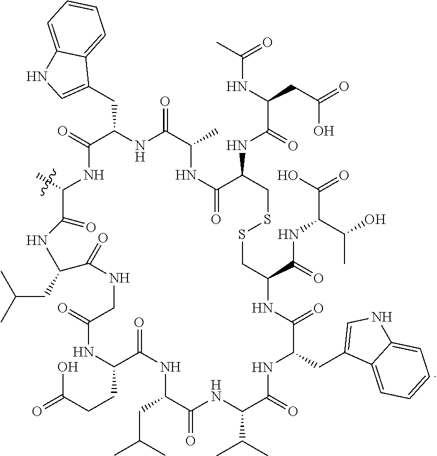 [SMILES: CC(=O)N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](C)NC(=O)[C@H](Cc2c[nH]c3ccccc23)NC(=O)[C@H](C)NC1=O ; "."]

In some embodiments, an antibody binding moiety, e.g.,

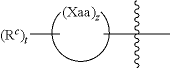

or R^{c}-(Xaa)z-, is or comprises

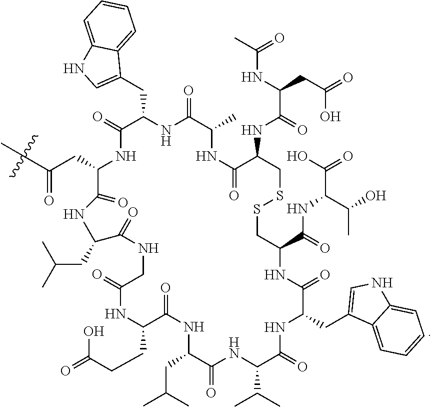 [SMILES: *C(=O)C[C@@H]1NC(=O)[C@H](Cc2c[nH]c3ccccc23)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC1=O]

In some embodiments, an antibody binding moiety, e.g.,

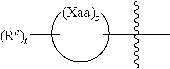

or R^{c}-(Xaa)z-, is or comprises

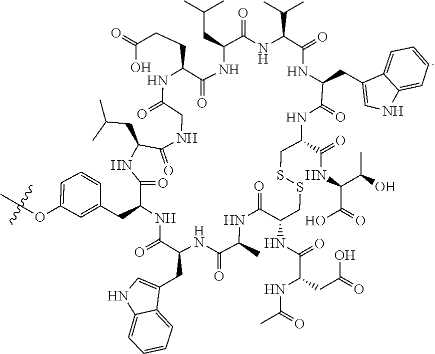 [SMILES: *Oc1cccc(C[C@@H]2NC(=O)[C@H](Cc3c[nH]c4ccccc34)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC2=O)c1]

In some embodiments, an antibody binding moiety, e.g.,

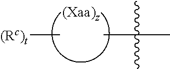

or R^{c}-(Xaa)z-, is or comprises

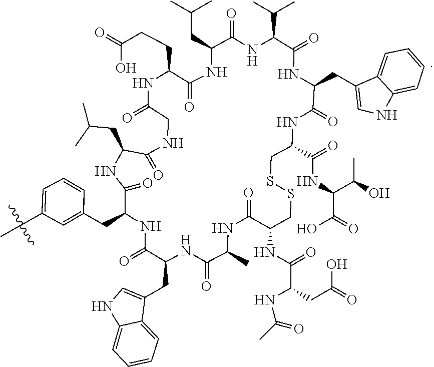 [SMILES: *c1cccc(C[C@@H]2NC(=O)[C@H](Cc3c[nH]c4ccccc34)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC2=O)c1]

In some embodiments, an antibody binding moiety,
e.g.,

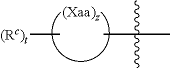

or R^{c}-(Xaa)z-, is or comprises

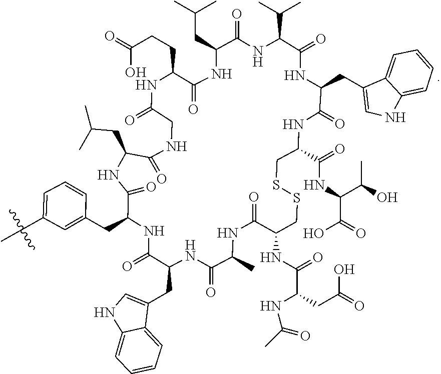 [SMILES: *c1cccc(C[C@@H]2NC(=O)[C@H](Cc3c[nH]c4ccccc34)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC2=O)c1]

In some embodiments, an antibody binding moiety, e.g.,

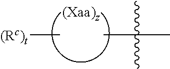

or R^{c}-(Xaa)z-, is or comprises

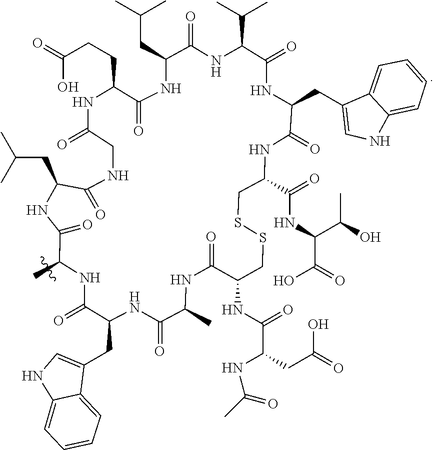 [SMILES: CC(=O)N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](C)NC(=O)[C@H](Cc2c[nH]c3ccccc23)NC(=O)[C@H](C)NC1=O]

In some embodiments, such antibody binding moieties are antibody binding moieties.

In some embodiments, an antibody binding moiety, e.g., R^{c}-(Xaa)z-, is or comprises a Z33 peptide moiety. In some embodiments, an antibody binding moiety, e.g., R^{c}-(Xaa)z-, is or comprises —FNMQQQRRFYEALHDPNLNEEQRNAKIKSIRDD-NH₂ or a fragment thereof. In some embodiments, an antibody binding moiety, e.g., R^{c}-(Xaa)z-, is or comprises FNMQCQRRFYEALHDPNLNEEQRNAKIKSIRDDC or a fragment thereof. In some embodiments, an antibody binding moiety, e.g.,

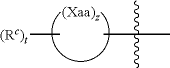

or R^{c}-(Xaa)z-, is or comprises a moiety of a peptide such as FNMQCQRRFYEALHDPNLNEEQRNAKIKSIRDDC, RGNCAYHRGQLVWCTYH, RGNCAYHKGQLVWCTYH, RGNCKYHRGQLVWCTYH, RGNCAWHRGKLVWCTYH, RGNCKWHRGELVWCTYH, RGNCKWHRGQLVWCTYH, RGNCKYHLGELVWCTYH, RGNCKYHLGQLVWCTYH, DCKWHLGELVWCT, DCKYHLGELVWCT, DCKWHRGELVWCT, DCKWHLGQLVWCT, DCKYHRGELVWCT, DCKYHLGQLVWCT, DCKWHRGQLVWCT, DCKYHRGQLVWCT, FNKQCQRRFYEALHDPNLNEEQRNARIRSIRDDC, FNMQCQRRFYEALHDPNLNEEQRNARIRSIKDDC, FNMQCQRRFYEALHDPNLNKEQRNARIRSIRDDC, FNMQCQRRFYEALHDPNLNEEQRNARIRSIRDDC, RGNCAWHLGQLVWCKYH, RGNCAWHLGELVWCKYH, RGNCAYHLGQLVWCTKH, RGNCAYHLGQLVWCTYK, RGNCAYHRGQLVWCTKH, KNMQCQRRFYEALHDPNLNEEQRNARIRSIRDDC, FNMQCQKRFYEALHDPNLNEEQRNARIRSIRDDC, FNMQCQRRFYEAKHDPNLNEEQRNARIRSIRDDC, FNMQCQRRFYEALHDPNLNEEQRKARIRSIRDDC, FNMQCQRRFYEALHDPNLNKEQRNARIRSIRDDC, FNMQCQRRFYEALHDPNLNEEQRNARIRSIKDDC, FNKQCQRRFYEALHDPNLNEEQRNARIRSIRDDC, FNMQCKRRFYEALHDPNLNEEQRNARIRSIRDDC, FNMQCQRRFYEALHDPNLNEEQRNARIRSIRKDC, Fc-III, FcBP-2, Fc-III-4C,

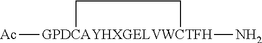

(X═K or R), etc., wherein two cysteine residues may optionally form a disulfide bond. In some embodiments, in a peptide described herein, two cysteine residues form a disulfide bond. In some embodiments, a peptide, such as Z33,

FNMQCQRRFYEALHDPNLNEEQRNAKIKSIRDDC,
RGNCAYHRGQLVWCTYH,
RGNCKYHRGQLVWCTYH,
RGNCAYHKGQLVWCTYH,
RGNCAWHRGKLVWCTYH,
RGNCKWHRGQLVWCTYH,
RGNCKWHRGELVWCTYH,
RGNCKYHLGELVWCTYH,
RGNCKYHLGQLVWCTYH,
DCKWHLGELVWCT,
DCKYHLGELVWCT,
DCKWHRGELVWCT,
DCKWHLGQLVWCT,
DCKYHRGELVWCT,
DCKYHLGQLVWCT,
DCKWHRGQLVWCT,
DCKYHRGQLVWCT,
FNKQCQRRFYEALHDPNLNEEQRNARIRSIRDDC,
FNMQCQRRFYEALHDPNLNEEQRNARIRSIKDDC,
FNMQCQRRFYEALHDPNLNKEQRNARIRSIRDDC,
FNMQCQRRFYEALHDPNLNEEQRNARIRSIRDDC,
RGNCAWHLGQLVWCKYH,
RGNCAWHLGELVWCKYH,
RGNCAYHLGQLVWCTKH,
RGNCAYHLGQLVWCTYK,
RGNCAYHRGQLVWCTKH,
KNMQCQRRFYEALHDPNLNEEQRNARIRSIRDDC,
FNMQCQKRFYEALHDPNLNEEQRNARIRSIRDDC,
FNMQCQRRFYEAKHDPNLNEEQRNARIRSIRDDC,
FNMQCQRRFYEALHDPNLNEEQRKARIRSIRDDC,
FNMQCQRRFYEALHDPNLNKEQRNARIRSIRDDC,
FNMQCQRRFYEALHDPNLNEEQRNARIRSIKDDC,
FNKQCQRRFYEALHDPNLNEEQRNARIRSIRDDC,
FNMQCKRRFYEALHDPNLNEEQRNARIRSIRDDC,
FNMQCQRRFYEALHDPNLNEEQRNARIRSIRKDC,
Fc-III, FcBP-2, Fc-III-4C,

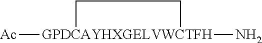

(X═K or R), etc., is connected through its N-terminus, C-terminus, or a side chain (e.g., of K (e.g., of underlined K residues in RGNCAYHKGQLVWCTYH, RGNCKYHRGQLVWCTYH, RGNCAWHRGKLVWCTYH, RGNCKWHRGELVWCTYH, RGNCKWHRGQLVWCTYH, RGNCKYHLGELVWCTYH, RGNCKYHLGQLVWCTYH, DCKWHLGELVWCT, DCKYHLGELVWCT, DCKWHRGELVWCT, DCKWHLGQLVWCT, DCKYHRGELVWCT, DCKYHLGQLVWCT, DCKWHRGQLVWCT, DCKYHRGQLVWCT, RGNCAWHLGQLVWCKYH, FNKQCQRRFYEALHDPNLNEEQRNARIRSIRDDC, FNMQCQRRFYEALHDPNLNEEQRNARIRSIKDDC, FNMQCQRRFYEALHDPNLNKEQRNARIRSIRDDC, RGNCAWHLGELVWCKYH, RGNCAYHLGQLVWCTKH, RGNCAYHLGQLVWCTYK, RGNCAYHRGQLVWCTKH, KNMQCQRRFYEALHDPNLNEEQRNARIRSIRDDC, FNMQCQKRFYEALHDPNLNEEQRNARIRSIRDDC, FNMQCQRRFYEAKHDPNLNEEQRNARIRSIRDDC, FNMQCQRRFYEALHDPNLNEEQRKARIRSIRDDC, FNMQCQRRFYEALHDPNLNKEQRNARIRSIRDDC, FNMQCQRRFYEALHDPNLNEEQRNARIRSIKDDC, FNKQCQRRFYEALHDPNLNEEQRNARIRSIRDDC, FNMQCKRRFYEALHDPNLNEEQRNARIRSIRDDC, FNMQCQRRFYEALHDPNLNEEQRNARIRSIRKDC, etc.)). In some embodiments, one or more amino acid residues of a sequence may be independently and optionally replaced (e.g., 1-5), deleted (e.g., 1-5) and/or inserted (e.g., 1-5) as described herein. In some embodiments, an antibody binding moiety, e.g.,

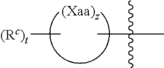

or R^{c}-(Xaa)z-, is or comprises —CXYHXXXLVWC—, —XCXYHXXXLVWC—, —CXYHXXXLVWCX—, —X₀₋₃CXYHXXXLVWCX₀₋₃—, —XCXYHXXXLVWCXXX—XXXCXYHXXXLVWCXXX—, wherein each X is independently an amino acid residue, and the two C residues optionally form a disulfide bond. In some embodiments, X³ (the X after H) is Orn. In some embodiments, X³ is Dab. In some embodiments, X³ is Lys(Ac). In some embodiments, X³ is Orn(Ac). In some embodiments, X³ is Dab(Ac). In some embodiments, X³ is Arg. In some embodiments, X³ is Ne. In some embodiments, X³ is Nva. In some embodiments, X³ is Val. In some embodiments, X³ is Tle. In some embodiments, X³ is Leu. In some embodiments, X³ is Ala(tBu). In some embodiments, X³ is Cha. In some embodiments, X³ is Phe. In some embodiments, an antibody binding moiety, e.g.,

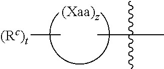

or R^{c}-(Xaa)z-, is or comprises DCAWHLGELVWCT. In some embodiments, a C-terminus and/or a N-terminus of a protein agent/peptide agent moiety are independently capped (e.g., RC(O)— such as CH₃C(O)— for N-terminus, —N(R′)₂ such as —NH₂ for C-terminus, etc.). In some embodiments, such antibody binding moieties are antibody binding moieties. In some embodiments, as described herein, a residue may be modified or replaced for connection with another moiety, e.g., in some embodiments, H may be replaced with an amino acid residue comprises a side chain that contain —COOH or a salt or activated form thereof (e.g., D).

In some embodiments, an antibody binding moiety, e.g.,

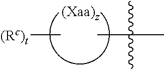

or R^{c}-(Xaa)z-, is or comprises (X₁₋₃)—C—(X₂)—H-(Xaa1)-G-(Xaa2)-L-V—W—C—(X₁₋₃), wherein each of X and Xaa is independently an amino acid residue and optionally not a cysteine residue. In some embodiments, Xaa1 is R, L, L, D, E, a 2-amino suberic acid residue, or a diaminopropionic acid residue. In some embodiments, Xaa2 is L, D, E, N, or Q. In some embodiments, Xaa1 is a lysine residue, a cysteine residue, an aspartic acid residue, a glutamic acid residue, a 2-amino suberic acid residue, or a diaminopropionic acid residue. In some embodiments, Xaa2 is a glutamic acid residue or an aspartic acid residue. In some embodiments, Xaa1 is an arginine residue or a leucine residue. In some embodiments, Xaa2 is a lysine residue, a glutamine residue, or an aspartic acid residue. In some embodiments, such antibody binding moieties are antibody binding moieties.

In some embodiments, an antibody binding moiety, e.g.,

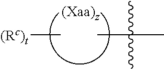

or R^{c}-(Xaa)z-, is or comprises (X¹⁻³)—C-(Xaa3)-(Xaa4)-H-(Xaa1)-G-(Xaa2)-L-V—W—C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such antibody binding moieties are antibody binding moieties.

In some embodiments, an antibody binding moiety, e.g.,

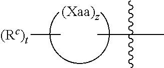

or R^{c}-(Xaa)z-, is or comprises D-C-(Xaa3)-(Xaa4)-H-(Xaa1)-G-(Xaa2)-L-V—W—C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such antibody binding moieties are antibody binding moieties.

In some embodiments, an antibody binding moiety, e.g.,

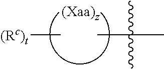

or R^{c}-(Xaa)z-, is or comprises D-C-(Xaa3)-(Xaa4)-H-(Xaa1)-G-(Xaa2)-L-V—W—C-T, wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, such antibody binding moieties are antibody binding moieties.

In some embodiments, an antibody binding moiety, e.g.,

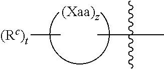

or R^{c}-(Xaa)z-, is or comprises R-G-N—C-(Xaa3)-(Xaa4)-H-(Xaa1)-G-(Xaa2)-L-V—W—C-(Xaa5)- (Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such antibody binding moieties are antibody binding moieties.

In some embodiments, antibody binding moieties, e.g., various antibody binding moieties described above, are protein binding moieties. In some embodiments, antibody binding moieties are antibody binding moieties. In some embodiments, LG is or comprises such an antibody binding moiety. In some embodiments, LG is or comprises a protein binding moiety. In some embodiments, LG is or comprises an antibody binding moiety.

In some embodiments, antibody binding moieties, e.g., antibody binding moieties, and useful technologies for developing and/or assessing such moieties are described in, e.g., Alves, Langmuir 2012, 28, 9640-9648; Choe et al., Materials 2016, 9, 994; doi:10.3390/ma9120994; Gupta et al., Nature Biomedical Engineering, vol. 3, 2019, 917-929; Muguruma, et al., ACS Omega 2019, 4, 14390-14397, doi: 10.1021/acsomega.9b01104; Yamada, et al., Angew Chem/nt Ed Engl. 2019 Apr. 16; 58(17):5592-5597, doi: 10.1002/anie.201814215; Kruljec, et al., Bioconjug Chem. 2017, 28(8): 2009-2030, doi: 10.1021/acs.bioconjchem.7b00335 (e.g., Fabsorbent, triazines, etc.); Kruljec, et al., Bioconjugate Chem. 2018, 29, 8, 2763-2775, doi: 10.1021/acs.bioconjchem.8b00395; WO2012017021A2, etc., the binding moieties (e.g., antibody binding moieties) of each of which is incorporated herein in its entirety by reference.

In some embodiments, an antibody binding moiety, e.g., a protein binding moiety (e.g., an antibody binding moiety), is an affinity substance described in AU 2018259856 or WO 2018199337, the affinity substance of each of which is incorporated herein by reference.

In some embodiments, an antibody binding moiety, e.g., an antibody binding moiety, is or comprises an adapter protein agent, e.g., as described in Hui, et al., Bioconjugate Chem. 2015, 26, 1456-1460, doi: 10.1021/acs.bioconjchem.5b00275. In some embodiments, when utilized in accordance with the present disclosure, adapter proteins do not require reactive residues (e.g., BPA) to achieve one or more or all advantages.

In some embodiments, antibody binding moiety, e.g., an antibody binding moiety is or comprises a triazine moiety, e.g., one described in US 2009/0286693. In some embodiments, an antibody binding moiety, e.g., an antibody binding moiety is of such a structure that its corresponding compound is a compound described in US 2009/0286693, the compounds of which are independently incorporated herein by reference. In some embodiments, an antibody binding moiety, e.g., an antibody binding moiety, is ABT. In some embodiments, ABT is of such a structure that H-ABT is a compound described in US 2009/0286693, the compounds of which are independently incorporated herein by reference. In some embodiments, such a compound can bind to an antibody. In some embodiments, such a compound can bind to Fc region of an antibody.

In some embodiments, an antibody binding moiety, e.g., an antibody binding moiety is or comprises a triazine moiety, e.g., one described in Teng, et al., A strategy for the generation of biomimetic ligands for affinity chromatography. Combinatorial synthesis and biological evaluation of an IgG binding ligand, J. Mol. Recognit. 1999; 12:67-75 (“Teng”). In some embodiments, an antibody binding moiety, e.g., an antibody binding moiety is of such a structure that its corresponding compound is a compound described in Teng, the compounds of which are independently incorporated herein by reference. In some embodiments, an antibody binding moiety, e.g., an antibody binding moiety, ABT is of such a structure that H-ABT is a compound described in Teng, the compounds of which are independently incorporated herein by reference. In some embodiments, such a compound can bind to an antibody. In some embodiments, such a compound can bind to Fc region of an antibody.

In some embodiments, an antibody binding moiety, e.g., an antibody binding moiety is a triazine moiety, e.g., one described in Uttamchandani, et al., Microarrays of Tagged Combinatorial Triazine Libraries in the Discovery of Small-Molecule Ligands of Human IgG, J Comb Chem. 2004 November-December; 6(6):862-8 (“Uttamchandani”). In some embodiments, an antibody binding moiety, e.g., an antibody binding moiety is of such a structure that its corresponding compound is a compound described in Uttamchandani, the compounds of which are independently incorporated herein by reference. In some embodiments, an antibody binding moiety, e.g., an antibody binding moiety, ABT is of such a structure that H-ABT is a compound described in Uttamchandani, the compounds of which are independently incorporated herein by reference. In some embodiments, such a compound can bind to an antibody. In some embodiments, such a compound can bind to Fc region of an antibody.

In some embodiments, an antibody binding moiety binds to one or more binding sites of protein A. In some embodiments, an antibody binding moiety binds to one or more binding sites of protein G. In some embodiments, an antibody binding moiety binds to one or more binding sites of protein L. In some embodiments, an antibody binding moiety binds to one or more binding sites of protein Z. In some embodiments, an antibody binding moiety binds to one or more binding sites of protein LG. In some embodiments, an antibody binding moiety binds to one or more binding sites of protein LA. In some embodiments, an antibody binding moiety binds to one or more binding sites of protein AG. In some embodiments, an antibody binding moiety is described in Choe, W., Durgannavar, T. A., & Chung, S. J. (2016). Fc-binding ligands of immunoglobulin G: An overview of high affinity proteins and peptides. Materials, 9(12). https://doi.org/10.3390/ma9120994.

In some embodiments, an antibody binding moiety, e.g., an antibody binding moiety can bind to a nucleotide-binding site. In some embodiments, an antibody binding moiety, e.g., an antibody binding moiety is a small molecule moiety that can bind to a nucleotide-binding site. In some embodiments, a small molecule is tryptamine. In some embodiments, an antibody binding moiety, e.g., an antibody binding moiety, ABT is of such a structure that H-ABT is tryptamine. Certain useful technologies were described in Mustafaoglu, et al., Antibody Purification via Affinity Membrane Chromatography Method Utilizing Nucleotide Binding Site Targeting With A Small Molecule, Analyst. 2016 November 28; 141(24): 6571-6582.

Many technologies are available for identifying and/or assessing and/or characterizing antibody binding moieties, including protein binding moieties (e.g., antibody binding moieties such as universal antibody binding moieties), and/or their utilization in provided technologies, e.g., those described in WO/2019/023501, the technologies of which are incorporated herein by reference. In some embodiments, an antibody binding moiety is a moiety (e.g., small molecule moiety, peptide moiety, nucleic acid moiety, etc.) that can selectively bind to IgG, and when used in provided technologies can provide and/or stimulate ADCC and/or ADCP. In some embodiments, peptide display technologies (e.g., phase display, non-cellular display, etc.) can be utilized to identify antibody binding moieties. In some embodiments, an antibody binding moiety is a moiety (e.g., small molecule moiety, peptide moiety, nucleic acid moiety, etc.) that can bind to IgG and optionally can compete with known antibody binders, e.g., protein A, protein G, protein L, etc.

As appreciated by those skilled in the art, antibodies of various properties and activities (e.g., antibodies recognizing different antigens, having optional modifications, etc.) may be targeted by antibody binding moieties described in the present disclosure. In some embodiments, such antibodies include antibodies administered to a subject, e.g., for therapeutic purposes. In some embodiments, antibody binding moieties described herein may bind antibodies toward different antigens and are useful for conjugating moieties of interest with various antibodies.

In some embodiments, an antibody binding moiety, e.g., an antibody binding moiety, is or comprises a meditope agent moiety. In some embodiments, a meditope agent is described in, e.g., US 2019/0111149.

In some embodiments, an antibody binding moiety, e.g., an antibody binding moiety, can bind to human IgG. In some embodiments, an antibody binding moiety, e.g., an antibody binding moiety, can bind to rabbit IgG. In some embodiments, an antibody binding moiety, e.g., an antibody binding moiety, binds to IgG1. In some embodiments, an antibody binding moiety, e.g., an antibody binding moiety, binds to IgG2. In some embodiments, an antibody binding moiety, e.g., an antibody binding moiety, binds to IgG3. In some embodiments, an antibody binding moiety, e.g., an antibody binding moiety, binds to IgG4. In some embodiments, an antibody binding moiety, e.g., an antibody binding moiety, binds to IgG1, IgG2 and/or IgG4. In some embodiments, an antibody binding moiety, e.g., an antibody binding moiety, binds to IgG1, IgG2 and IgG4.

In some embodiments,

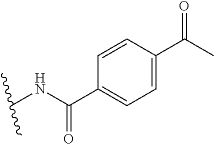 [SMILES: *NC(=O)c1ccc(C(C)=O)cc1]

is utilized in a reference technology as a non-antibody binding moiety. In some embodiments, CH₃— is utilized in a reference technology a non-antibody binding moiety. In some embodiments, CH₃C(O)— is utilized in a reference technology a non-antibody binding moiety. In some embodiments, CH₃C(O)NH— is utilized in a reference technology a non-antibody binding moiety. In some embodiments, CH₃C(O)NHCH₂— is utilized in a reference technology a non-antibody binding moiety. In some embodiments, CH₃CH₂— is utilized in a reference technology a non-antibody binding moiety. In some embodiments, CH₃CH₂NH— is utilized in a reference technology a non-antibody binding moiety. In some embodiments, CH₃CH₂NHC(O)— is utilized in a reference technology a non-antibody binding moiety.

In some embodiments, antibody binding moieties (e.g., antibody binding moieties) bind to targets (e.g., antibody agents for antibody binding moieties) with a Kd that is about 1 mM-1 pM or less. In some embodiments, a Kd is about 1 mM, 0.5 mM, 0.2 mM, 0.1 mM, 0.05 mM, 0.02 mM, 0.01 mM, 0.005 mM, 0.002 mM, 0.001 mM, 500 nM, 200 nM, 100 nM, 50 nM, 20 nM, 10 nM, 5 nM, 2 nM, 1 nM, 0.5 nM, 0.2 nM, 0.1 nM, or less. In some embodiments, Kd is about 1 mM or less. In some embodiments, Kd is about 0.5 mM or less. In some embodiments, Kd is about 0.1 mM or less. In some embodiments, Kd is about 0.05 mM or less. In some embodiments, Kd is about 0.01 mM or less. In some embodiments, Kd is about 0.005 mM or less. In some embodiments, Kd is about 0.001 mM or less. In some embodiments, Kd is about 500 nM or less. In some embodiments, Kd is about 200 nM or less. In some embodiments, Kd is about 100 nM or less. In some embodiments, Kd is about 50 nM or less. In some embodiments, Kd is about 20 nM or less. In some embodiments, Kd is about 10 nM or less. In some embodiments, Kd is about 5 nM or less. In some embodiments, Kd is about 2 nM or less. In some embodiments, Kd is about 1 nM or less. For example, in some embodiments, antibody binding moieties bind to IgG antibody agents with Kd described herein.

As appreciated by those skilled in the art, antibodies of various properties and activities (e.g., antibodies recognizing different antigens, having optional modifications, etc.) may be recruited by antibody binding moieties described in the present disclosure. In some embodiments, such antibodies include antibodies administered to a subject, e.g., for therapeutic purposes. In some embodiments, antibodies recruited by antibody binding moieties comprise antibodies toward different antigens. In some embodiments, antibodies recruited by antibody binding moieties comprise antibodies whose antigens are not present on the surface or cell membrane of target cells. In some embodiments, antibodies recruited by antibody binding moieties comprise antibodies which are not targeting antigens present on surface or cell membrane of targets. In some embodiments, antigens on surface of target cells may interfere with the structure, conformation, and/or one or more properties and/or activities of recruited antibodies which bind such antigens. In some embodiments, as appreciated by those skilled in the art, provided technologies comprise universal antibody binding moieties which recruit antibodies of diverse specificities, and no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of recruited antibodies are toward the same antigen, protein, lipid, carbohydrate, etc. Among other things, one advantage of the present disclosure is that provided technologies comprising universal antibody binding moieties can utilize diverse pools of antibodies such as those present in serum. In some embodiments, universal antibody binding moieties of the present disclosure (e.g., those in ARMs) are contacted with a plurality of antibodies, wherein no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of the plurality of antibodies are toward the same antigen, protein, lipid, carbohydrate, etc. In some embodiments, recruited antibodies are those in IVIG. In some embodiments, IVIG may be administered prior to, concurrently with or subsequently to an agent or composition. Among other things, antibodies of various types of immunoglobulin structures may be recruited. In some embodiments, one or more subclasses of IgG are recruited. In some embodiments, recruited antibodies comprise IgG1. In some embodiments, recruited antibodies comprise IgG2. In some embodiments, recruited antibodies comprise IgG3. In some embodiments, recruited antibodies comprise IgG4. In some embodiments, recruited antibodies are or comprise IgG1 and IgG2. In some embodiments, recruited antibodies are or comprise IgG1, IgG2 and IgG4. In some embodiments, recruited antibodies are or comprise IgG1, IgG2, IgG3 and IgG4. Recruited antibodies may interact various types of receptors, e.g., those expressed by various types of immune cells. In some embodiments, recruited antibodies can effectively interact various types of Fc receptors and provide desired immune activities. In some embodiments, recruited antibodies can recruit immune cells. In some embodiments, recruited antibodies can effectively interact with hFcγRIIIA. In some embodiments, recruited antibodies can effectively interact with hFcγRIIIA on macrophages. In some embodiments, macrophages are recruited to provide ADCC and/or ADCP activities toward a virus, e.g., a SARS-CoV-2 virus, and/or cells infected thereby. In some embodiments, NK cells are recruited to provide immune activities. In some embodiments, recruited antibodies can effectively interact with hFcγRIIA. In some embodiments, recruited antibodies can effectively interact with hFcγRIIA on dendritic cells. In some embodiments, antibody moieties in agents of the present disclosure comprise one or more properties, structures and/or activities of recruited antibodies described herein.

### Amino Acids

In some embodiments, provided compounds and agents may comprise one or more amino acid moieties, e.g., in antibody binding moieties, linker moieties, etc. Amino acid moieties can either be those of natural amino acids or unnatural amino acids. In some embodiments, an amino acid has the structure of formula A-I:

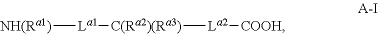

or a salt thereof, wherein:
   each of R^{a1}, R^{a2} and R^{a3} is independently -L^{a}-R′ or an amino acid side chain;
   each of L^{a1} and L^{a2} is independently L^{a};
   each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
   each R′ is independently —R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
   two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, an amino acid residue, e.g., of an amino acid having the structure of formula A-I, has the structure of —N(R^{a1})-L^{a1}-C(R^{a2})(R^{a3})-L^{a2}-CO—. In some embodiments, each amino acid residue in a peptide independently has the structure of —N(R^{a1})-L^{a1}-C(R^{a2})(R^{a3})-L^{a2}-CO—.

In some embodiments, the present disclosure provides a derivative of an amino acid of formula A-I or a salt thereof. In some embodiments, a derivative is an ester. In some embodiments, the present disclosure provides a compound of formula NH(R^{a1})-L^{a1}-C(R^{a2})(R^{a3})-L^{a2}-COORCT salt thereof, wherein R^{CT} is R′ and each other variable is independently as described herein. In some embodiments, R^{CT} is R. In some embodiments, R^{CT} is optionally substituted aliphatic. In some embodiments, R^{CT} is t-butyl.

In some embodiments, L^{a1} is a covalent bond. In some embodiments, a compound of formula A-I is of the structure NH(R^{a1})—C(R^{a2})(R^{a3})-L^{a2}-COOH. In some embodiments, L^{a2} is —CH₂SCH₂—.

In some embodiments, L^{a2} is a covalent bond. In some embodiments, a compound of formula A-I is of the structure NH(R^{a1})-L^{a1}-C(R^{a2})(R^{a3})—COOH. In some embodiments, an amino acid residue has the structure of —N(R^{a1})-L^{a1}-C(R^{a2})(R^{a3})—CO—. In some embodiments, L^{a1} is —CH₂CH₂S—. In some embodiments, L^{a1} is —CH₂CH₂S—, wherein the CH₂ is bonded to NH(R^{a1}).

In some embodiments, L^{a1} is a covalent bond and L^{a2} is a covalent bond. In some embodiments, a compound of formula A-I is of the structure NH(R^{a1})—C(R^{a2})(R^{a3})—COOH. In some embodiments, a compound of formula A-I is of the structure NH(R^{a1})—CH(R^{a2})—COOH. In some embodiments, a compound of formula A-I is of the structure NH(R^{a1})—CH(R^{a3})—COOH. In some embodiments, a compound of formula A-I is of the structure NH₂—CH(R^{a2})—COOH. In some embodiments, a compound of formula A-I is of the structure NH₂—CH(R^{a3})—COOH. In some embodiments, an amino acid residue has the structure of —N(R^{a1})—C(R^{a2})(R^{a3})—CO—. In some embodiments, an amino acid residue has the structure of —N(R^{a1})—CH(R^{a2})—CO—. In some embodiments, an amino acid residue has the structure of —N(R^{a1})—CH(R^{a3})—CO—. In some embodiments, an amino acid residue has the structure of —NH—CH(R^{a2})—CO—. In some embodiments, an amino acid residue has the structure of —NH—CH(R^{a3})—CO—.

In some embodiments, L^{a} is a covalent bond. In some embodiments, L^{a} is optionally substituted C₁₋₆ bivalent aliphatic. In some embodiments, L^{a} is optionally substituted C₁₋₆ alkylene. In some embodiments, L^{a} is —CH₂—. In some embodiments, L^{a} is —CH₂CH₂—. In some embodiments, L^{a} is —CH₂CH₂CH₂—.

In some embodiments, L^{a} is bivalent optionally substituted C₁₋₂₀ aliphatic, wherein one or more methylene units are independently replaced with —C(O)—, —N(R′)—, -Cy-, and/or —O—. In some embodiments, L^{a} is bivalent optionally substituted C₁₋₂₀ aliphatic, wherein one or more methylene units are independently replaced with —C(O)N(R′)—, -Cy-, and —O—. In some embodiments, L^{a} is bivalent optionally substituted C₁₋₂₀ aliphatic, wherein two or more methylene units are independently replaced with —C(O)N(R′)—, and -Cy- in addition to other optional replacements. In some embodiments, -Cy- is optionally substituted. In some embodiments, -Cy- is optionally substituted with an electron-withdrawing group as described herein. In some embodiments, -Cy- is substituted with one or more —F. In some embodiments, -Cy- is optionally substituted 1,3-phenylene. In some embodiments, -Cy- is optionally substituted 1,4-phenylene. In some embodiments, L^{a} is or comprises

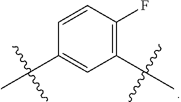 [SMILES: *c1ccc(F)c(*)c1]

In some embodiments, L^{a} is or comprises

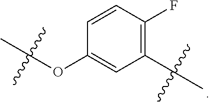 [SMILES: *Oc1ccc(F)c(*)c1]

In some embodiments, L^{a} is or comprises

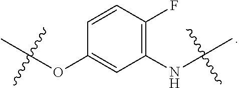 [SMILES: *Nc1cc(O*)ccc1F]

In some embodiments, L^{a} is or comprises

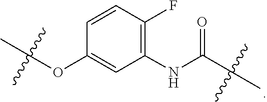 [SMILES: *Oc1ccc(F)c(NC(*)=O)c1]

In some embodiments, L^{a} is or comprises

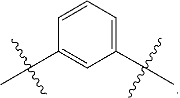 [SMILES: *c1cccc(*)c1]

In some embodiments, L^{a} is or comprises

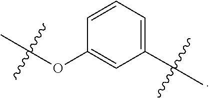 [SMILES: *Oc1cccc(*)c1]

In some embodiments, L^{a} is or comprises

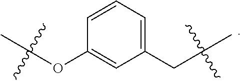 [SMILES: *Cc1cccc(O*)c1]

In some embodiments, L^{a} is or comprises

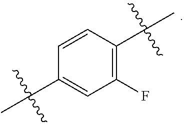 [SMILES: *c1ccc(*)c(F)c1]

In some embodiments, L^{a} is or comprises

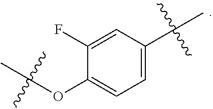 [SMILES: *Oc1ccc(*)cc1F]

In some embodiments, L^{a} is or comprises

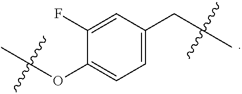 [SMILES: *Cc1ccc(O*)c(F)c1]

In some embodiments, L^{a} is or comprises

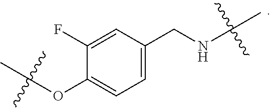 [SMILES: *NCc1ccc(O*)c(F)c1]

In some embodiments, L^{a} is or comprises

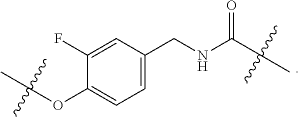 [SMILES: *Oc1ccc(CNC(*)=O)cc1F]

In some embodiments, L^{a} is or comprises

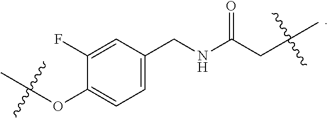 [SMILES: *CC(=O)NCc1ccc(O*)c(F)c1]

In some embodiments, L^{a} is or comprises

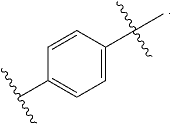 [SMILES: *c1ccc(*)cc1]

In some embodiments, L^{a} is or comprises

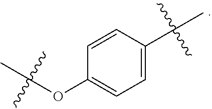 [SMILES: *Oc1ccc(*)cc1]

In some embodiments, L^{a} is or comprises

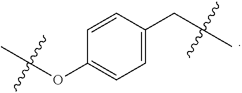 [SMILES: *Cc1ccc(O*)cc1]

In some embodiments, L^{a} is or comprises

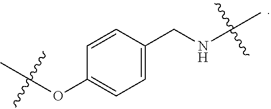 [SMILES: *NCc1ccc(O*)cc1]

In some embodiments, L^{a} is or comprises

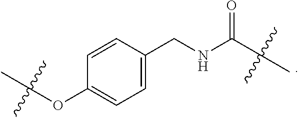 [SMILES: *Oc1ccc(CNC(*)=O)cc1]

In some embodiments, L^{a} is or comprises

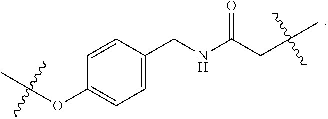 [SMILES: *CC(=O)NCc1ccc(O*)cc1]

In some embodiments, R′ is R. In some embodiments, R^{a1} is R, wherein R is as described in the present disclosure. In some embodiments, R^{a1} is R, wherein R methyl. In some embodiments, R² is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a1} is a protective group. In some embodiments, R^{a1} is -Fmoc. In some embodiments, R^{a1} is -Dde.

In some embodiments, each of R^{a1}, R^{a2} and R^{a3} is independently -L^{a}-R′.

In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen. In some embodiments, R² is -L^{a}-R and R^{a3} is —H. In some embodiments, R^{a3} is -L^{a}-R and R² is —H. In some embodiments, R² is —CH₂—R and R^{a3} is —H. In some embodiments, R^{a3} is —CH₂—R and R^{a2} is —H. In some embodiments, R^{a2} is R and R^{a3} is —H. In some embodiments, R^{a3} is R and R^{a2} is —H.

In some embodiments, R^{a2} is -L^{a}-R, wherein R is as described in the present disclosure. In some embodiments, R^{a2} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is -L^{a}-R, wherein R is as described in the present disclosure. In some embodiments, R^{a3} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a3} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a3} is a side chain of an amino acid. In some embodiments, R^{a3} is a side chain of a standard amino acid.

In some embodiments, one or R^{a2} and R^{a3} is —H. In some embodiments, one or R^{a2} and R^{a3} is -L^{a}-R, wherein L^{a} is as described herein. In some embodiments, L^{a} is not a covalent bond. In some embodiments, one or more methylene units of L^{a} are independently and optionally replaced as described herein, e.g., with —C(O)—, —N(R′)—, —O—, —C(O)—N(R′)— and/or -Cy-, etc. In some embodiments, L^{a} is or comprises —C(O)—, —N(R′)— and -Cy-. In some embodiments, L^{a} is or comprises —C(O)N(R′)— and -Cy-. In some embodiments, as described herein, -Cy- is substituted and one or more substituents are independently an electron-withdrawing group.

In some embodiments, an amino acid side chain is R^{a2} or R^{a3}. In some embodiments, an amino acid side chain is or comprises -L^{LG1}-L^{LG2}-L^{LG3}-L^{LG4}-H. In some embodiments, an amino acid side chain is or comprises -L^{LG2}-L^{LG3}-L^{LG4}-H. In some embodiments, an amino acid side chain is or comprises -L^{LG3}-L^{LG4}-H. In some embodiments, an amino acid side chain is or comprises -L^{LG4}-H. In some embodiments, such a side chain is

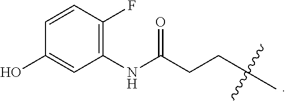 [SMILES: *CCC(=O)Nc1cc(O)ccc1F]

In some embodiments, such a side chain is

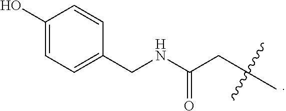 [SMILES: *CC(=O)NCc1ccc(O)cc1]

In some embodiments, such a side chain

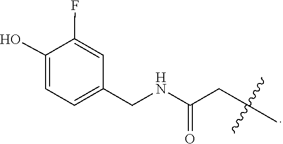 [SMILES: *CC(=O)NCc1ccc(O)c(F)c1]

In some embodiments, such a side chain is

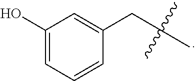 [SMILES: *Cc1cccc(O)c1]

In some embodiments, R is an optionally substituted C₁₋₆ aliphatic. In some embodiments, R is an optionally substituted C₁₋₆ alkyl. In some embodiments, R is —CH₃. In some embodiments, R is optionally substituted pentyl. In some embodiments, R is n-pentyl.

In some embodiments, R is a cyclic group. In some embodiments, R is an optionally substituted C₃₋₃₀ cycloaliphatic group. In some embodiments, R is cyclopropyl.

In some embodiments, R is an optionally substituted aromatic group, and an amino acid residue of an amino acid of formula A-I is a Xaa^{A}. In some embodiments, R^{a2} or R^{a3} is —CH₂—R, wherein R is an optionally substituted aryl or heteroaryl group. In some embodiments, R is optionally substituted phenyl. In some embodiments, R is phenyl. In some embodiments, R is optionally substituted phenyl. In some embodiments, R is 4-trifluoromethylphenyl. In some embodiments, R is 4-phenylphenyl. In some embodiments, R is optionally substituted 5-30 membered heteroaryl having 1-10 heteroatoms independently selected from oxygen, nitrogen, sulfur, phosphorus and silicon. In some embodiments, R is optionally substituted 5-14 membered heteroaryl having 1-5 heteroatoms independently selected from oxygen, nitrogen, and sulfur. In some embodiments, R is

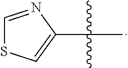 [SMILES: *c1cscn1]

In some embodiments, R is optionally substituted pyridinyl. In some embodiments, R is 1-pyridinyl. In some embodiments, R is 2-pyridinyl. In some embodiments, R is 3-pyridinyl. In some embodiments, R is

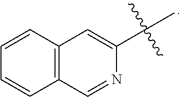 [SMILES: *c1cc2ccccc2cn1]

In some embodiments, R′ is-COOH. In some embodiments, a compound of and an amino acid residue of an amino acid of formula A-I is a Xaa^{N}.

In some embodiments, R′ is —NH₂. In some embodiments, a compound of an amino acid residue of an amino acid of formula A-I is a Xaa^{P}.

In some embodiments, R^{a2} or R^{a3} is R, wherein R is C₁₋₂₀ aliphatic as described in the present disclosure. In some embodiments, a compound of an amino acid residue of an amino acid of formula A-I is a Xaa^{H}. In some embodiments, R is —CH₃. In some embodiments, R is ethyl. In some embodiments, R is propyl. In some embodiments, R is n-propyl. In some embodiments, R is butyl. In some embodiments, R is n-butyl. In some embodiments, R is pentyl. In some embodiments, R is n-pentyl. In some embodiments, R is cyclopropyl.

In some embodiments, two or more of R^{a1}, R^{a2}, and R^{a3} are R and are taken together to form an optionally substituted ring as described in the present disclosure.

In some embodiments, R^{a1} and one of R^{a2} and R^{a3} are R and are taken together to form an optionally substituted 3-6 membered ring having no additional ring heteroatom other than the nitrogen atom to which R^{a1} is bonded to. In some embodiments, a formed ring is a 5-membered ring as in proline.

In some embodiments, R^{a2} and R^{a3} are R and are taken together to form an optionally substituted 3-6 membered ring as described in the present disclosure. In some embodiments, R^{a2} and R^{a3} are R and are taken together to form an optionally substituted 3-6 membered ring having one or more nitrogen ring atom. In some embodiments, R^{a2} and R^{a3} are R and are taken together to form an optionally substituted 3-6 membered ring having one and no more than one ring heteroatom which is a nitrogen atom. In some embodiments, a ring is a saturated ring.

In some embodiments, an amino acid is a natural amino acid. In some embodiments, an amino acid is an unnatural amino acid. In some embodiments, an amino acid is an alpha-amino acid. In some embodiments, an amino acid is a beta-amino acid. In some embodiments, a compound of formula A-I is a natural amino acid. In some embodiments, a compound of formula A-I is an unnatural amino acid.

In some embodiments, an amino acid comprises a hydrophobic side chain. In some embodiments, an amino acid with a hydrophobic side chain is A, V, I, L, M, F, Y or W. In some embodiments, an amino acid with a hydrophobic side chain is A, V, I, L, M, or F. In some embodiments, an amino acid with a hydrophobic side chain is A, V, I, L, or M. In some embodiments, an amino acid with a hydrophobic side chain is A, V, I, or L. In some embodiments, a hydrophobic side chain is R wherein R is C₁₋₁₀ aliphatic. In some embodiments, R is C₁₋₁₀ alkyl. In some embodiments, R is methyl. In some embodiments, R is ethyl. In some embodiments, R is propyl. In some embodiments, R is butyl. In some embodiments, R is pentyl. In some embodiments, R is n-pentyl. In some embodiments, an amino acid with a hydrophobic side chain is NH₂CH(CH₂CH₂CH₂CH₂CH₃)COOH. In some embodiments, an amino acid with a hydrophobic side chain is (S)—NH₂CH(CH₂CH₂CH₂CH₂CH₃)COOH. In some embodiments, an amino acid with a hydrophobic side chain is (R)—NH₂CH(CH₂CH₂CH₂CH₂CH₃)COOH. In some embodiments, a hydrophobic side chain is —CH₂R wherein R is optionally substituted phenyl. In some embodiments, R is phenyl. In some embodiments, R is phenyl substituted with one or more hydrocarbon group. In some embodiments, R is 4-phenylphenyl. In some embodiments, an amino acid with a hydrophobic side chain is NH₂CH(CH₂₋₄-phenylphenyl)COOH. In some embodiments, an amino acid with a hydrophobic side chain is (S)—NH₂CH(CH₂₋₄-phenylphenyl)COOH. In some embodiments, an amino acid with a hydrophobic side chain is (R)—NH₂CH(CH₂₋₄-phenylphenyl)COOH.

In some embodiments, an amino acid comprises a positively charged side chain (e.g., at physiological pH) as described herein. In some embodiments, such an amino acid comprises a basic nitrogen in its side chain. In some embodiments, such an amino acid is Arg, His or Lys. In some embodiments, such an amino acid is Arg. In some embodiments, such an amino acid is His. In some embodiments, such an amino acid is Lys.

In some embodiments, an amino acid comprises a negatively charged side chain (e.g., at physiological pH) as described herein. In some embodiments, such an amino acid comprises a —COOH in its side chain. In some embodiments, such an amino acid is Asp. In some embodiments, such an amino acid is Glu.

In some embodiments, an amino acid comprises a side chain comprising an aromatic group as described herein. In some embodiments, such an amino acid is Phe, Tyr, Trp, or His. In some embodiments, such an amino acid is Phe. In some embodiments, such an amino acid is Tyr. In some embodiments, such an amino acid is Trp. In some embodiments, such an amino acid is His. In some embodiments, such an amino acid is NH₂—CH(CH₂₋₄-phenylphenyl)-COOH. In some embodiments, such an amino acid is (S)—NH₂—CH(CH₂₋₄-phenylphenyl)-COOH. In some embodiments, such an amino acid is (R)—NH₂—CH(CH₂₋₄-phenylphenyl)-COOH.

In some embodiments, an amino acid is

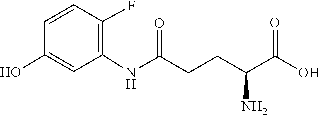 [SMILES: N[C@@H](CCC(=O)Nc1cc(O)ccc1F)C(=O)O]

or a salt thereof. In some embodiments, an amino acid is

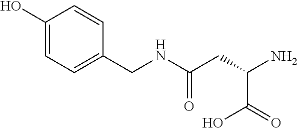 [SMILES: N[C@@H](CC(=O)NCc1ccc(O)cc1)C(=O)O]

or a salt thereof. In some embodiments, an amino acid is

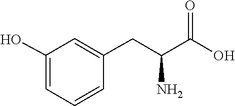 [SMILES: N[C@@H](Cc1cccc(O)c1)C(=O)O]

or a salt thereof. In some embodiments, an amino acid is

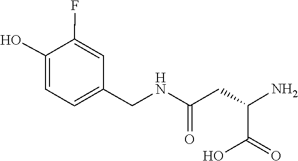 [SMILES: N[C@@H](CC(=O)NCc1ccc(O)c(F)c1)C(=O)O]

or a salt thereof. In some embodiments, an amino acid is

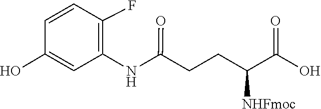

or a salt thereof. In some embodiments, an amino acid is

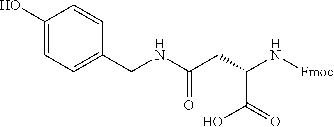

or a salt thereof. In some embodiments, an amino acid is

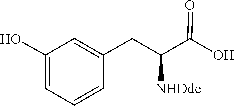

or a salt thereof. In some embodiments, an amino acid is

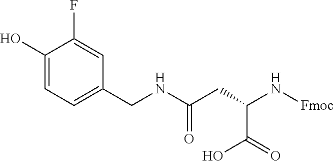

or a salt thereof. In some embodiments, a provided compound is

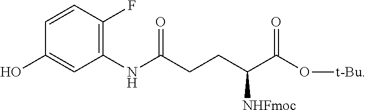

In some embodiments, the present disclosure provides polypeptide agents comprising one or more amino acid residues described in the present disclosure.

### General Methods of Providing Various Agents

Agents of the present disclosure may be prepared or isolated in general by synthetic and/or semi-synthetic methods or recombinant methods in accordance with the present disclosure. Certain technologies are described in the Examples. In some embodiments, polypeptide agents, e.g., cellular receptor binding moiety peptide agents, maybe be prepared using biological expression systems. In some embodiments, provided agents are prepared synthetically. In some embodiments, provided agents are prepared using certain technologies described in WO2019/023501, which is incorporated herein in its entirety by reference.

Various technologies, e.g., those for preparing antibody-drug conjugates, may be utilized in preparation of MATE agents. In many such technologies, conjugation is not selective with respect to amino acid residue sites, and product compositions usually contain various different types of agents which may differ from each other with respect to number of target binding moieties conjugated and/or conjugation sites. In some embodiments, the present disclosure provides technologies that can be utilized for selective conjugation of target binding moieties at particular amino acid residue sites.

In some embodiments, the present disclosure provides a method, comprising: contacting a first agent comprising a cellular receptor binding moiety linked to a first reactive group optionally through a first linker with a second agent comprising an antibody moiety linked to a second reactive group optionally through a second linker, wherein the first reactive group reacts with a second reactive group, and forming a product agent comprising a cellular receptor binding moiety and an antibody binding moiety optionally through a linker.

In some embodiments, the present disclosure provides a method comprising:
contacting a first composition comprising a plurality of first agents each independently comprising a cellular receptor binding moiety linked to a first reactive group optionally through a first linker moiety with a second composition comprising a plurality of second agents each independently comprising an antibody moiety optionally linked to a second reactive group optionally through a second linker moiety,
wherein a product composition comprising a plurality of product agents each independently comprising a cellular receptor binding moiety and an antibody binding moiety optionally through a linker is formed.

In some embodiments, a first composition is a composition comprising a first agent as described herein. In some embodiments, second agents independently comprise second reactive groups. In some embodiments, a second composition is a composition comprising a plurality of agents as described herein, wherein each cellular receptor binding moiety is independently a reactive group as described herein. In some embodiments, a second composition is an antibody composition, wherein antibodies in the composition are not chemically modified. In some embodiments, a second composition is an IVIG preparation. In some embodiments, a product composition is a composition comprising a plurality of agents as described herein, wherein each cellular receptor binding moiety is independently a cellular receptor binding moiety as described herein.

In some embodiments, a cellular receptor binding moiety in a product agent is a cellular receptor binding moiety in a first agent. In some embodiments, an antibody moiety in a product agent is an antibody moiety in a second agent. In some embodiments, a second agent is an antibody agent, e.g., a monoclonal antibody, an antibody in a polyclonal antibody, an antibody in an IVIG preparation, etc. In some embodiments, a second reactive group is a function group of an amino acid residue, e.g., —NH2 of Lys, —SH of Cys, etc. In some embodiments, a second reactive group is —NH2 of a Lys residue, e.g., of a residue selected from K246 and K248 of an IgG1 heavy chain amino acid residues corresponding thereto, K251 and K253 of an IgG2 heavy chain and amino acid residues corresponding thereto, and K239 and K241 of an IgG4 heavy chain and amino acid residues corresponding thereto. In some embodiments, the present disclosure provides selective reactions at particular amino acid residues of antibody moieties.

In some embodiments, a second reactive group is installed to an antibody moiety optionally through a linker. In some embodiments, a second reactive group is installed to an antibody moiety through a linker. In some embodiments, a second reactive group is selectively linked to certain location(s) of an antibody moiety, e.g., certain location(s) selected from K246 and K248 of an IgG1 heavy chain amino acid residues corresponding thereto, K251 and K253 of an IgG2 heavy chain and amino acid residues corresponding thereto, and K239 and K241 of an IgG4 heavy chain and amino acid residues corresponding thereto. In some embodiments, the present disclosure provides selective reactions at particular amino acid residues of antibody moieties.

In some embodiments, the present disclosure provides agents each independently comprising an antibody binding moiety that binds to an antibody agent, a reactive group, a cellular receptor binding moiety, and optionally one or more linker moieties linking such groups/moieties. In some embodiments, such agents are useful as reaction partners (e.g., first agents) for conjugating moieties of interest, e.g., target binding moieties, reactive groups (e.g., second reactive groups) to agents comprising antibody moieties (e.g., second agents). In some embodiments, the present disclosure provides agents for conjugating moieties of interest to antibody moieties in various agents or antibody agents (e.g., monoclonal antibody agents, polyclonal antibody agents, antibody agents of IVIG preparations, etc.). In some embodiments, provided agents each comprise a cellular receptor binding moiety, a reactive group, an antibody binding moiety, and optionally one or more linker moieties (linkers) linking such moieties. In some embodiments, an antibody binding moiety is part of a leaving group that is released upon contacting such an agent (e.g., a first agent) with an antibody moiety (e.g., of a second agent) and reacting a reactive group of such an agent (e.g., a first reactive group of a first agent) with a reactive group of an antibody moiety (e.g., a second reactive group of a second agent, such as —NH2 of a Lys residue of an antibody protein). In some embodiments, provided technologies among other things can provide improved conjugation efficiency, high selectivity, and/or fewer steps (in some cases, single step) to conjugation product agents. In some embodiments, a provided agent, e.g., a first agent, is a compound of formula R-I or a salt thereof:

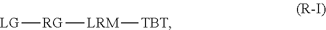

or a salt thereof, wherein:
LG is a group comprising an antibody binding moiety;
RG is a reactive group;
LRM is a linker; and
TBT is a cellular receptor binding moiety.

In some embodiments, LG is or comprises an antibody binding moiety as described herein, and a linker which links an antibody binding moiety and RG.

As used in the present disclosure, a moiety generally refers to a part of a molecule, e.g., in an ester RCOOR′, the alcohol moiety is RO—. In some embodiments, a moiety of an agent (e.g., a target agent, a peptide agent, an antibody agent, etc.) retains one or more or all desirable structural features, properties, functions, and/or activities of a compound. For example, in some embodiments, a cellular receptor binding moiety can bind to a target, optionally in a comparable fashion, as its corresponding target binding agent; in some embodiments, a target agent moiety maintains one or more desired structural features, properties, functions, and/or properties comparable to its corresponding target agent; in some embodiments, an antibody agent moiety maintains one or more desired structural features, properties, functions, and/or properties (e.g., 3-dimension structure, antigen specificity, antigen-binding capacity, and/or immunological functions, etc.) comparable to its corresponding antibody agent. In some embodiments, a moiety of an agent, e.g., a target agent moiety, a peptide agent moiety, an antibody agent moiety, etc. is a monovalent (for a monovalent moiety), bivalent (for a bivalent moiety), or polyvalent (for a polyvalent moiety) radical of an agent, e.g., a target agent (for a target agent moiety), a peptide agent (for a peptide agent moiety), an antibody agent (for an antibody agent moiety), etc. In some embodiments, a monovalent radical is formed by removing a monovalent part (e.g., hydrogen, halogen, another monovalent group like alkyl, aryl, etc.) from a compound/agent. In some embodiments, a bivalent or polyvalent radical is formed by removing one or more monovalent (e.g., hydrogen, halogen, monovalent groups like alkyl, aryl, etc.), bivalent and/or polyvalent parts from a compound/agent. In some embodiments, radicals are formed by removing hydrogen atoms. In some embodiments, a moiety is monovalent. In some embodiments, a moiety is bivalent. In some embodiments, a moiety is polyvalent.

In some embodiments, LG is or comprises R^{LG}-L^{LG}-, wherein R^{LG} is or comprises an antibody binding moiety, and L^{LG} is a linker moiety as described herein. In some embodiments, LG is ABT-L^{LG}-. In some embodiments, L^{LG} is -L^{LG}1-L^{LG2}-, wherein each of L^{LG1} and L^{LG2} is independently a linker moiety as described herein. In some embodiments, L^{LG} is -L^{LG}1-L^{LG2}-L^{LG3}-, wherein each of L^{LG1}, L^{LG2} and L^{LG3} is independently as linker moiety described herein. In some embodiments, L^{LG} is L^{LG1}-L^{LG2}-L^{LG3}-L^{LG4}- wherein each of L^{LG11} L^{LG2}, L^{LG3} and L^{LG4} is independently a linker moiety as described herein. In some embodiments, L^{LG1} is bonded to R^{LG}. In some embodiments, L^{LG1} is bonded to cellular receptor binding moiety. In some embodiments, L^{LG} is -L^{LG1}-, and a reactive group comprises L^{LG2}, L^{LG3} and L^{LG4}. In some embodiments, L^{LG} is -L^{LG1}-L^{LG2}-, and a reactive group comprises L^{LG3} and L^{LG4}. In some embodiments, L^{LG} is -L^{LG1}-L^{LG2}-L^{LG3}-, and a reactive group comprises L^{LG4}. In some embodiments, each of L^{LG1}, L^{LG2} L^{LG3} and L^{LG4} is independently L as described herein.

In some embodiments, antibody binding moieties, LG, etc. are released after reactions, e.g., after first agents (e.g., wherein MOIs are target binding moieties) react with second agents (e.g., which are antibody agents comprising reactive amino acid residues such as amino groups as second reactive groups and/or second agents comprising second reactive groups introduced to antibody agents), or after first agents (e.g., wherein MOIs are reactive groups such as second reactive groups) react with second agents which are antibody agents. In some embodiments, an antibody binding moiety is released after a reaction. In some embodiments, LG is released after a reaction. In some embodiments, a leaving group is released as part of a compound having the structure of LG-H or a salt thereof. In some embodiments, an antibody binding moiety is released as part of a compound having the structure of LG-H or a salt thereof. In some embodiments, LG is released as part of a compound having the structure of LG-H or a salt thereof. In some embodiments, a released compound has the structure of R^{LG}-L^{LG1}-L^{LG2}-L^{LG3}-L^{LG4}-H or a salt thereof. In some embodiments, an antibody binding moiety is released as part of a compound having the structure of R^{LG}-L^{LG1}-L^{LG2}-L^{LG3}-L^{LG4}-H or a salt thereof. In some embodiments, an antibody binding moiety is released as part of a compound having the structure of R^{LG}-L^{LG1}-L^{LG2}-L^{LG3}-L^{LG4}-H or a salt thereof, wherein R^{LG} is or comprises an antibody binding moiety. In some embodiments, LG is released as part of a compound having the structure of R^{LG}-L^{LG1}-L^{LG2}-L^{LG3}-L^{LG4}-H or a salt thereof, wherein LG is R^{LG}-L^{LG}, and L^{LG} is -L^{LG1}-, -L^{LG1}-L^{LG2} L^{LG1}L^{LG2}L^{LG3} or L^{LG1}-L^{LG2}L^{LG3}L^{LG4} In some embodiments, LG is released as part of a compound having the structure of R^{LG}-L^{LG1}-L^{LG2}-L^{LG3}-L^{LG4}-H or a salt thereof, wherein LG is R^{LG}-L^{LG}1-. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}. In some embodiments, LG is released as part of a compound having the structure of R^{LG}-L^{LG1}-L^{LG2}-L^{LG3}-L^{LG4}-H or a salt thereof, wherein LG is R^{LG}-L^{LG}1-L^{LG2}-L^{LG3}. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}-L^{LG3}-L^{LG4}.

In some embodiments, L is a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂—)ₙ]—, wherein n is 1-20. In some embodiments, L is a covalent bond, or a bivalent optionally substituted, linear or branched C₁₋₁₀₀ aliphatic or heteroaliphatic group 1-20 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, or -[(—O—C(R′)₂—C(R′)₂—)ₙ]—, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, amino acid residues, or -[(—O—C(R′)₂—C(R′)₂—)ₙ]—, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, amino acid residues, or -[(—O—C(R′)₂—C(R′)₂—)n]—, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, amino acid residues, or -[(—O—C(R′)₂—C(R′)₂—)ₙ]—, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or -[(—O—C(R′)₂—C(R′)₂—)ₙ]—, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, -Cy-, or —[(—O—C(R′)₂—C(R′)₂—)ₙ]—, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or —[(—O—C(R′)₂—C(R′)₂—)ₙ]—, wherein n is 1-10. In some embodiments, L comprises no —C(O)O—. In some embodiments, L comprises no —C(O)—N(R′)—. In some embodiments, L comprises no —S—. In some embodiments, L comprises no —S-Cy-. In some embodiments, L comprises no —S—S—. In some embodiments, L does not contain one or more or any of —C(O)O—, —C(O)—N(R′)—, —S—, and —S—S—. In some embodiments, L does not contain one or more or any of —C(O)O—, —C(O)—N(R′)—, —S-Cy-, and —S—S—. In some embodiments, L does not contain one or more or any of —C(O)O—, —S—, and —S—S—. In some embodiments, L does not contain one or more or any of —C(O)O—, —S-Cy-, and —S—S—. In some embodiments, L contains none of —C(O)O—, —S—, and —S—S—. In some embodiments, L contains none of —C(O)O—, —S-Cy-, and —S—S—. In some embodiments, L contains none of —C(O)O— and —S—S—.

In some embodiments, L is a covalent bond. In some embodiments, L is not a covalent bond.

In some embodiments, L^{LG1} is a covalent bond. In some embodiments, L^{LG1} is not a covalent bond. In some embodiments, L^{LG1} is or comprises —(CH₂CH₂O)n-. In some embodiments, L^{LG1} is or comprises —(CH₂)n-O—(CH₂CH₂O)n-(CH₂)n-, wherein each n is independently as described herein, and each —CH₂— is independently optionally substituted. In some embodiments, L^{LG1} is —(CH₂)n-O—(CH₂CH₂O)n-(CH₂)n-, wherein each n is independently as described herein, and each —CH₂— is independently optionally substituted. In some embodiments, L^{LG1} is —(CH₂)₂—O—(CH₂CH₂O)n-(CH₂)₂—, wherein n is as described herein, and each —CH₂— is independently optionally substituted. In some embodiments, L^{LG1} is —(CH₂)₂—O—(CH₂CH₂O)n-(CH₂)₂—, wherein n is as described herein.

In some embodiments, L^{LG1} is —CH₂—. In some embodiments, L^{LG1} is —(CH₂)₂—. In some embodiments, L^{LG1} is —(CH₂)₂—C(O)—. In some embodiments, L^{LG1} is —(CH₂)₂—C(O)—NH—. In some embodiments, L^{LG1} is —(CH₂)₃—. In some embodiments, L^{LG1} is —(CH₂)₃NH—. In some embodiments, L^{LG1} is —(CH₂)₃NH—C(O)—. In some embodiments, L^{LG1} is —C(O)—(CH₂)₃NH—C(O)—. In some embodiments, L^{LG1} is —C(O)—(CH₂)₃—. In some embodiments, L^{LG1} is —NH—C(O)—(CH₂)₃—. In some embodiments, L^{LG1} is —NHC(O)—(CH₂)₃NH—C(O)—. In some embodiments, a —CH₂— is bonded to an antibody binding moiety.

In some embodiments, L^{LG1} is —CH₂CH₂—O—CH₂CH₂—O—CH₂CH₂—. In some embodiments, L^{LG1} is —CH₂CH₂—O—CH₂CH₂—O—CH₂CH₂—C(O)—. In some embodiments, L^{LG1} is —CH₂CH₂—O—CH₂CH₂—O—CH₂CH₂—C(O)NH—. In some embodiments, L^{LG1} is —CH₂CH₂—O—CH₂CH₂—O—CH₂CH₂—C(O)NH—CH₂—. In some embodiments, —CH₂CH₂— is bonded to an antibody binding moiety.

In some embodiments, L^{LG1} is —(CH₂CH₂O)n-. In some embodiments, L^{LG1} is —(CH₂CH₂O)n-CH₂—CH₂—. In some embodiments, L^{LG1} is —(CH₂CH₂O)n-CH₂—CH₂—C(O)—. In some embodiments, L^{LG1} is —(CH₂CH₂O)₂—CH₂—CH₂—C(O)—. In some embodiments, L^{LG1} is —(CH₂CH₂O)₄—CH₂—CH₂—C(O)—. In some embodiments, L^{LG1} is —(CH₂CH₂O)₃—CH₂—CH₂—C(O)—. In some embodiments, —C(O)— is bonded to an antibody binding moiety.

In some embodiments, L^{LG1} is —N(R′)—. In some embodiments, L^{LG1} is —NH—. In some embodiments, L^{LG1} is —NH—[(—CH₂CH₂—O-)]n-. In some embodiments, L^{LG1} is —NH—[(—CH₂CH₂—O-)]n-CH₂CH₂—. In some embodiments, L^{LG1} is —NH—[(—CH₂CH₂—O-)]n-CH₂CH₂—NH—. In some embodiments, L^{LG1} is —NH—[(—CH₂CH₂—O-)]n-CH₂CH₂—NH—C(O)—. In some embodiments, n is 1. In some embodiments, n is 2. In some embodiments, n is 3. In some embodiments, n is 4. In some embodiments, n is 5. In some embodiments, L^{LG1} is —NH—CH₂CH₂—O—. In some embodiments, L^{LG1} is —NH—CH₂CH₂—O—CH₂CH₂—. In some embodiments, L^{LG1} is —NH—CH₂CH₂—O—CH₂CH₂—NH—. In some embodiments, L^{LG1} is —NH—CH₂CH₂—O—CH₂CH₂—NH—C(O)—.

In some embodiments, L^{LG1} is NH—[(—CH₂CH₂—O—)]₂—. In some embodiments, L^{LG1} is —NH—[(—CH₂CH₂—O—)]₂—CH₂CH₂—. In some embodiments, L^{LG1} is —NH—[(—CH₂CH₂—O—)]₂—CH₂CH₂—NH—. In some embodiments, L^{LG1} is —NH—[(—CH₂CH₂—O—)]₂—CH₂CH₂—NH—C(O)—.

In some embodiments, L^{LG1} is NH—[(—CH₂CH₂—O—)]₃—. In some embodiments, L^{LG1} is —NH—[(—CH₂CH₂—O—)]₃—CH₂CH₂—. In some embodiments, L^{LG1} is —NH—[(—CH₂CH₂—O—)]₃—CH₂CH₂—NH—. In some embodiments, L^{LG1} is —NH—[(—CH₂CH₂—O—)]₃—CH₂CH₂—NH—C(O)—. In some embodiments, L^{LG1} is —NH—[(—CH₂CH₂—O—)]₄-. In some embodiments, L^{LG1} is —NH—[(—CH₂CH₂—O—)]₄—CH₂CH₂—. In some embodiments, L^{LG1} is —NH—[(—CH₂CH₂—O—)]₄—CH₂CH₂—NH—. In some embodiments, L^{LG1} is —NH—[(—CH₂CH₂—O—)]₄—CH₂CH₂—NH—C(O)—. In some embodiments, L^{LG1} is —NH—[(—CH₂CH₂—O—)]₅-. In some embodiments, L^{LG1} is —NH—[(—CH₂CH₂—O—)]₅—CH₂CH₂—. In some embodiments, L^{LG1} is —NH—[(—CH₂CH₂—O—)]₅—CH₂CH₂—NH—. In some embodiments, L^{LG1} is —NH—[(—CH₂CH₂—O—)]₅—CH₂CH₂—NH—C(O)—. In some embodiments, —NH— is bonded to an antibody binding moiety.

In some embodiments, L^{LG1} is —CH₂—. In some embodiments, L^{LG1} is —CH₂CH₂—. In some embodiments, L^{LG1} is —CH₂CH₂NH—. In some embodiments, L^{LG1} is —CH₂CH₂NH—(CO)—. In some embodiments, —CH₂— is bonded to an antibody binding moiety.

In some embodiments, L^{LG1} is —CH₂—. In some embodiments, L^{LG1} is —CH₂C(O)—. In some embodiments, L^{LG1} is —CH₂C(O)NH—. In some embodiments, L^{LG1} is —CH₂(CO)NHCH₂—. In some embodiments, —CH₂—C(O)— is bonded to an antibody binding moiety at —CH₂—.

In some embodiments, L^{LG2} is a covalent bond. In some embodiments, L^{LG2} is not a covalent bond. In some embodiments, L^{LG2} is —N(R′)C(O)—. In some embodiments, L^{LG2} is —NHC(O)—. In some embodiments, L^{LG2} is —(CH₂)n-N(R′)C(O)—, wherein —(CH₂)n- is optionally substituted. In some embodiments, L^{LG2} is —(CH₂)n-OC(O)—, wherein —(CH₂)n- is optionally substituted. In some embodiments, L^{LG2} is —(CH₂)n-OC(O)N(R′)—, wherein —(CH₂)n- is optionally substituted. In some embodiments, L^{LG2} is —(CH₂)n-OC(O)NH—, wherein —(CH₂)n- is optionally substituted. In some embodiments, n is 1-10, e.g., 1, 2, 3, 4, 5, 6, 7, 8, 9, or 10. In some embodiments, n is 1. In some embodiments, n is 2. In some embodiments, n is 3. In some embodiments, —(CH₂)n- is substituted. In some embodiments, —(CH₂)n- is unsubstituted. In some embodiments, L^{LG2} is —CH₂N(CH₂CH₂CH₂S(O)₂OH)—C(O)—. In some embodiments, L^{LG2} is —C(O)—NHCH₂—. In some embodiments, L^{LG2} is —C(O)—NHCH₂CH₂—. In some embodiments, L^{LG}2 is —C(O)O—CH₂—. In some embodiments, L^{LG2} is —NH—C(O)O—CH₂—. In some embodiments, —C(O)— is bonded to L^{LG3}. In some embodiments, —N(R′)—, —NH—, or an optionally substituted —CH₂— unit (of optionally substituted —(CH₂)n-) is bonded to L^{LG3}. In some embodiments L^{LG2} is —NH—, —NHC(O)—, —(CH₂)n-NHC(O)—, —(CH₂)n-OC(O)—, —(CH₂)n-OC(O)NH—, —C(O)—NHCH₂—, —C(O)—NHCH₂CH₂—, —C(O)O—CH₂—, or —NH—C(O)O—CH₂—.

In some embodiments, L^{LG2} is —N(R′)—. In some embodiments, L^{LG}2 is —N(R)—. In some embodiments, L^{LG2} is —NH—.

In some embodiments, L^{LG2} is optionally substituted bivalent C₁₋₆ aliphatic. In some embodiments, L^{LG2} is —CH₂—. In some embodiments, L^{LG2} is —CH₂NH—. In some embodiments, L^{LG2} is —CH₂NH—C(O)—. In some embodiments, L^{LG2} is —CH₂NH—C(O)—CH₂—.

In some embodiments, L^{LG3} is or comprises an optionally substituted aryl ring. In some embodiments, L^{LG3} is or comprises an optionally substituted phenyl ring. In some embodiments, L^{LG3} is a phenyl ring substituted with one or more electron-withdrawing groups. As appreciated by those skilled in the art, various electron-withdrawing groups are known in the art and may be utilized in accordance with the present disclosure. In some embodiments, an electron-withdrawing group is halogen. In some embodiments, an electron-withdrawing group is —F. In some embodiments, it is —C₁. In some embodiments, it is —Br. In some embodiments, it is —I. In some embodiments, an electron-withdrawing group comprises an X═Y double bond, wherein X is bonded to the group to which the electron-withdrawing group is a substituent, and at least one of X and Y is a heteroatom. In some embodiments, X is a heteroatom. In some embodiments, Y is a heteroatom. In some embodiments, each of X and Y is independently a heteroatom. In some embodiments, Y is 0. In some embodiments, Y is S. In some embodiments, X is C. In some embodiments, X is N. In some embodiments, X is P. In some embodiments, X is S. In some embodiments, X═Y is C═O. In some embodiments, X═Y is N═O. In some embodiments, X═Y is S═O. In some embodiments, X═Y is P═O. In some embodiments, an electron-withdrawing group is —C(O)-L-R′. In some embodiments, an electron-withdrawing group is —C(O)—R′. In some embodiments, it is —NO₂. In some embodiments, it is —S(O)-L-R′. In some embodiments, it is —S(O)—R′. In some embodiments, it is —S(O)₂-L-R′. In some embodiments, it is —S(O)₂—O—R′. In some embodiments, it is —S(O)₂—N(R′)₂. In some embodiments, it is —P(O)(-L-R′)₂. In some embodiments, it is —P(O)(R′)₂. In some embodiments, it is —P(O)(OR′)₂. In some embodiments, it is —P(O)[N(R′)₂]₂.

In some embodiments, L^{LG3} is -L^{LG3a}-L^{LG3b}-, wherein L^{LG3a} is a covalent bond or —C(O)O—CH₂—, wherein —CH₂— is optionally substituted, and L^{LG3b} is an optionally substituted aryl ring. In some embodiments, L^{LG3a} is bonded to L^{LG2}, and L^{LG3b} is bonded to L^{LG4}.

In some embodiments, L^{LG3a} is a covalent bond. In some embodiments, L^{LG3a} is —C(O)O—CH₂—, wherein —CH₂— is optionally substituted. In some embodiments, L^{LG3a} is —C(O)O—CH₂—, wherein —CH₂— is substituted. In some embodiments, L^{LG3a} is —C(O)O—CH₂—, wherein —CH₂— is unsubstituted.

In some embodiments, a first group, an antibody binding moiety, and/or LG is released as part of a compound having the structure of R^{LG}-L^{LG1}-L^{LG2}-H or a salt thereof.

In some embodiments, L^{LG3b} is an optionally substituted phenyl ring. In some embodiments, at least one substituent is an electron-withdrawing group as described herein.

In some embodiments, L^{LG3} is

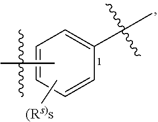

wherein s is 0-4, each R⁵ is independently halogen, —NO₂, -L-R′, —C(O)-L-R′, —S(O)-L-R′, —S(O)₂-L-R′, or —P(O)(-L-R′)₂. In some embodiments, C₁ is bonded to L^{LG4}. In some embodiments, L^{LG3} is

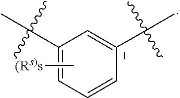

In some embodiments, L^{LG3} is

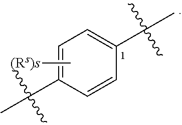

In some embodiments, L^{LG3} is

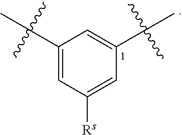

In some embodiments, L^{LG3} is

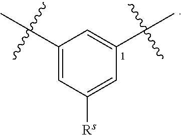

In some embodiments, L^{LG3} is

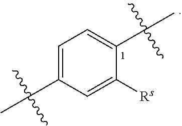

In some embodiments, L^{LG3} is

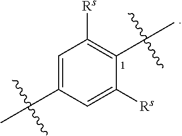

In some embodiments, L^{LG3b} is

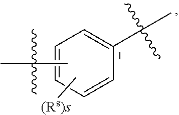

wherein s is 0-4, each R⁵ is independently halogen, —NO₂, -L-R′, —C(O)-L-R′, —S(O)-L-R′, —S(O)₂-L-R′, or —P(O)(-L-R′)₂. In some embodiments, C₁ is bonded to L^{LG4}. In some embodiments, L^{LG3b} is

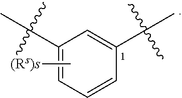

In some embodiments, L^{LG3b} is

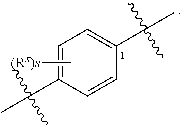

In some embodiments, L^{LG3b} is

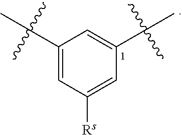

In some embodiments, L^{LG3b} is

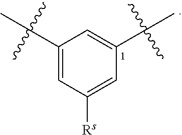

In some embodiments, L^{LG3b} is

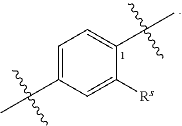

In some embodiments, L^{LG3b} is

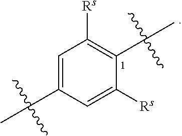

In some embodiments, s is 0. In some embodiments, s is 1-4. In some embodiments, s is 1. In some embodiments, s is 2. In some embodiments, s is 3. In some embodiments, s is 4.

In some embodiments, s is 1-4, and at least one R⁵ is an electron-withdrawing group, e.g., an electron-withdrawing group described above. In some embodiments, at least one R⁵ is —NO₂. In some embodiments, at least one R⁵ is —F. In some embodiments, each R⁵ is independently an electron-withdrawing group. In some embodiments, each R⁵ is —NO₂. In some embodiments, each R⁵ is —F.

In some embodiments, an electron-withdrawing group or R⁵ is at C2. In some embodiments, an electron-withdrawing group or R⁵ is at C3. In some embodiments, an electron-withdrawing group or R⁵ is at C4. In some embodiments, an electron-withdrawing group or R⁵ is at C2 and C5.

In some embodiments, L^{LG3} is

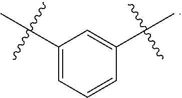 [SMILES: *c1cccc(*)c1]

In some embodiments, L^{LG3} is

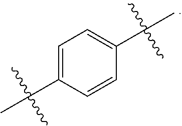 [SMILES: *c1ccc(*)cc1]

In some embodiments, L^{LG3} is

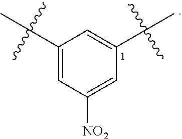 [SMILES: *c1cc(*)cc([N+](=O)[O-])c1]

In some embodiments, L^{LG3} is

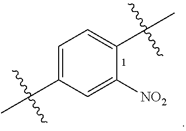 [SMILES: *c1ccc(*)c([N+](=O)[O-])c1]

In some embodiments, L^{LG3} is

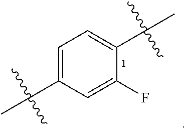 [SMILES: *c1ccc(*)c(F)c1]

In some embodiments, L^{LG3} is

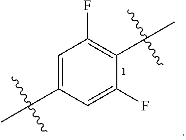 [SMILES: *c1cc(F)c(*)c(F)c1]

In some embodiments, L^{LG3} is

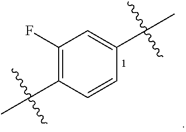 [SMILES: *c1ccc(*)c(F)c1]

In some embodiments, L^{LG3} is

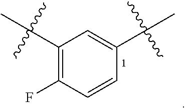 [SMILES: *c1ccc(F)c(*)c1]

In some embodiments, L^{LG3b} is

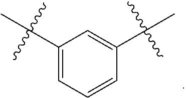 [SMILES: *c1cccc(*)c1]

In some embodiments, L^{LG3b} is

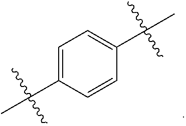 [SMILES: *c1ccc(*)cc1]

In some embodiments, L^{LG3b} is

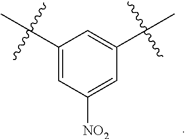 [SMILES: *c1cc(*)cc([N+](=O)[O-])c1]

In some embodiments, L^{LG3b} is

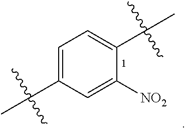 [SMILES: *c1ccc(*)c([N+](=O)[O-])c1]

In some embodiments, L^{LG3b} is

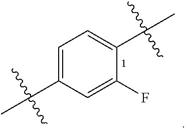 [SMILES: *c1ccc(*)c(F)c1]

In some embodiments, L^{LG3b} is

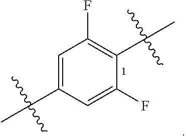 [SMILES: *c1cc(F)c(*)c(F)c1]

In some embodiments, L^{LG3b} is

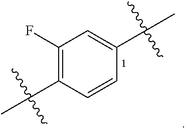 [SMILES: *c1ccc(*)c(F)c1]

In some embodiments, L^{LG3b} is

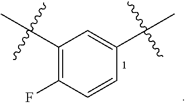 [SMILES: *c1ccc(F)c(*)c1]

In some embodiments, L^{LG3b} is optionally substituted

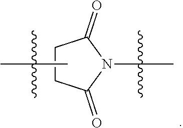

In some embodiments, the nitrogen atom is bound to L^{LG4} which is —O—. In some embodiments, the nitrogen atom is bound to L^{LG4} which is —O—, and -L^{RG1}-L^{RG2}- is —C(O)—.

In some embodiments, -L^{LG4}-L^{RG1}-L^{RG2}- is —O—C(O)—. In some embodiments, -L^{LG4}-L^{RG1}-L^{RG2}- is —S—C(O)—.

In some embodiments, L^{LG4} is a covalent bond. In some embodiments, L^{LG4} is not a covalent bond. In some embodiments, L^{LG4} is —O—. In some embodiments, L^{LG4} is —N(R′)—. In some embodiments, L^{LG4} is —NH—. In some embodiments, L^{LG4} is —N(CH₃)—. In some embodiments, L^{LG4} is —N(R′)—, and L^{LG3} is —O—. In some embodiments, R′ is optionally substituted C₁₋₆ alkyl. In some embodiments, L^{LG4} is —S—.

As described herein, in some embodiments, R^{LG} is or comprises an antibody binding moiety. In some embodiments, R^{LG} is or comprises a protein binding moiety. In some embodiments, R^{LG} is or comprises an antibody binding moiety. In some embodiments, R^{LG} is an antibody binding moiety. In some embodiments, R^{LG} is a protein binding moiety. In some embodiments, R^{LG} is an antibody binding moiety.

In some embodiments, R^{LG} is

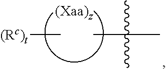

R^{c}-(Xaa)z-, a nucleic acid moiety, or a small molecule moiety. In some embodiments, R^{LG} is or comprises

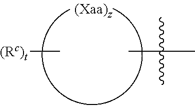

as described herein. In some embodiments, R^{LG} is or comprises R^{c}-(Xaa)z- as described herein. In some embodiments, R^{LG} is or comprises a small molecule moiety. In some embodiments, R^{LG} is or comprises a peptide agent. In some embodiments, R^{LG} is or comprises a nucleic acid agent. In some embodiments, R^{LG} is or comprises an aptamer agent. In some embodiments, an antibody binding moiety is or comprises

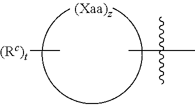

as described herein. In some embodiments, a protein binding moiety is or comprises

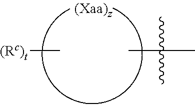

as described herein. In some embodiments, an antibody binding moiety is or comprises

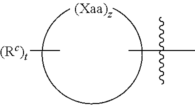

as described herein. In some embodiments, an antibody binding moiety is or comprises R^{c}-(Xaa)z- as described herein. In some embodiments, a protein binding moiety is or comprises R^{c}-(Xaa)z- as described herein. In some embodiments, an antibody binding moiety is or comprises R^{c}-(Xaa)z- as described herein.

In some embodiments, target binding moieties may be conjugated to antibody moieties optionally through linker moieties utilizing technologies described in US 2020/0190165 in accordance with the present disclosure.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g. mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that provided agents may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

As appreciated by those skilled in the art, reaction partners are generally contacted with each other under conditions and for a time sufficient for production of the desired results, e.g., formation of product agents and compositions thereof to desired extents. Many reaction conditions/reaction times may be assessed and utilized if they are suitable for desired purposes in accordance with the present disclosure; certain such conditions, reaction times, assessment, etc. are described in the Examples.

In some embodiments, an agent formed, e.g., a product MATE agent, has the structure of formula M-I or M-II, or a salt thereof. In some embodiments, a cellular receptor binding moiety in a product agent (e.g., a MATE agent) is the same as a cellular receptor binding moiety in a reaction partner (e.g., a first agent comprising a cellular receptor binding moiety) utilized to prepare a product agent. In some embodiments, an antibody moiety in a product agent (e.g., a MATE agent) is the same as an antibody moiety in a reaction partner (e.g., a second agent comprising an antibody moiety) utilized to prepare a product agent.

In some embodiments, linker moieties (or a part thereof) connected to target binding moieties and/or antibody moieties may be transferred from reaction partners (e.g., L^{RM} of formula R-I or a salt thereof). In some embodiments, a linker moiety in a product agent (may be referred to as L^{PM}; e.g., L in formula M-I or M-II) is or comprises a linker moiety in a reaction partner (e.g., one between a reactive group and a cellular receptor binding moiety, e.g., L^{RM}). In some embodiments, L^{PM} is or comprises L^{RM}. In some embodiments, L^{PM} is -L^{RM}-L^{RG2}-. In some embodiments, L^{RG2} is —C(O)—. In some embodiments, L^{RG2} is —C(O)—, and is bonded to —NH— of a target agent moiety, e.g., —NH— in a side chain of a lysine residue of a protein moiety, which in some embodiments, is an antibody moiety.

Reaction partners, e.g., compounds of formula R-I or salts thereof, typically do not contain moieties that can react with reactive groups under conditions under which reactive groups react with target agents. In some embodiments, to the extent that some moieties in reaction partners may react with reactive groups under conditions under which reactive groups react with target agents, reactions between such moieties and reactive groups are significantly slower and/or less efficient compared to reactions between reactive groups and target agents. In some embodiments, reactions between such moieties and reactive groups do not significantly reduce (e.g., no more than about 5%, 10%, 15%, 20%, 25%, 30%, 40%, 50%, 60%, 70%, 80%, 90%, etc. of reduction) efficiencies, yields, rates, and/or conversions, etc., of reactions between reactive groups and target agents. In some embodiments, reactive groups (e.g., ester groups, activated carboxylic acid derivatives, etc.) react with amino groups (e.g., —NH₂ groups) of target agents (e.g., protein agents such as antibody agents). In some embodiments, reaction partners, e.g., compounds of formula R-I or salts thereof, do not contain amine groups. In some embodiments, compounds of formula R-I or salts thereof (or portions thereof, such as R^{LG}, L^{LG}, L^{LG1}, L^{LG2}, L^{LG3}, L^{LG4}, L^{RG1}, L^{RG2}, L^{RM}, and/or MOI) do not contain amine groups. In some embodiments, they do not contain primary amine groups (—NH₂). In some embodiments, they do not contain —CH₂NH₂. In some embodiments, they do not contain —CH₂CH₂NH₂. In some embodiments, they do not contain —CH₂CH₂CH₂NH₂. In some embodiments, they do not contain —CH₂CH₂CH₂CH₂NH₂. In some embodiments, amine groups, e.g., primary amine groups, are capped (e.g., by introduction of acyl groups (e.g., R—C(O)— (e.g., acetyl)) to form amide groups) to prevent or reduce undesired reactions.

In some embodiments, reactions are performed in buffer systems. In some embodiments, buffer systems of present disclosure maintains structures and/or functions of target agents, cellular receptor binding moiety, etc. In some embodiments, a buffer is a phosphate buffer. In some embodiments, a buffer is a PBS buffer. In some embodiments, a buffer is a borate buffer. In some embodiments, buffers of the present disclosure provide and optionally maintain certain pH value or range. For example, in some embodiments, a useful pH is about 7-9, e.g., 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 9.0, etc. In some embodiments, a pH is 7.4. In some embodiments, a pH is 7.5. In some embodiments, a pH is 7.8. In some embodiments, a pH is 8.0. In some embodiments, a pH is 8.2. In some embodiments, a pH is 8.3.

Provided technologies can provide various advantages. Among other things, in some embodiments, connection of a cellular receptor binding moiety in a reaction partner (e.g., a compound comprising a reactive group located between an antibody binding moiety and a cellular receptor binding moiety (e.g., a compound of formula R-I or a salt thereof)) to an agent comprising an antibody moiety (e.g., a second agent such as an antibody agent) and release of an antibody binding moiety in a provided reaction partner can be achieved in one reaction and/or in one pot. Thus, in many embodiments, no separate reactions/steps are performed to remove antibody binding moieties. As appreciated by those skilled in the art, by performing connection of cellular receptor binding moiety and release of antibody binding moiety in a single reaction/operation, provided technologies can avoid separate steps for antibody binding moiety removal and can improve overall efficiency (e.g., by simplify operations, increasing overall yield, etc.), reduce manufacturing cost, improve product purity (e.g., by avoiding exposure to antibody binding moiety removal conditions, which typically involve one or more of reduction, oxidation, hydrolysis (e.g., of ester groups), etc., conditions and may damage target agent moieties (e.g., for protein agent moieties, protein amino acid residues, overall structures, and/or post-translational modifications (e.g., glycans of antibodies) thereof. In some embodiments, provided technologies among other things can provided improved efficiency (e.g., in terms of reaction rates and/or conversion percentages), increased yield, increased purity/homogeneity, and/or enhanced selectivity, particularly compared to reference technologies wherein a reaction partner containing no antibody binding moieties is used, without introducing step(s) for antibody binding moiety removal (e.g., antibody binding moiety is removed in the same step as cellular receptor binding moiety conjugation).

In some embodiments, the present disclosure provides products of provided processes, which, among other things, contain low levels of damage to antibody moieties compared to processes comprising steps which are performed for antibody binding moiety removal but not for substantial conjugation of moieties of interest (e.g. target binding moieties). In some embodiments, provided product agent compositions have high homogeneity (e.g., with respect to the number of cellular receptor binding moiety per antibody moiety, and/or positions of amino acid residues in antibody moieties conjugated to moieties of interest) compared to reference product compositions (e.g., those from technologies without using antibody binding moieties, or utilizing extra step(s) for antibody binding moiety removal (e.g., not utilizing reaction partners described herein which comprise a reactive group located between an antibody binding moiety and a cellular receptor binding moiety).

In some embodiments, the present disclosure provides a product agent which is an agent comprising an antibody moiety, a cellular receptor binding moiety and optionally a linker moiety linking an antibody binding moiety and a cellular receptor binding moiety. In some embodiments, the present disclosure provides compositions of such agents.

In some embodiments, the present disclosure provides a composition comprising a plurality of agents, wherein each agent independently comprises:
an antibody moiety,
a cellular receptor binding moiety, and
optionally a linker moiety linking an antibody binding moiety and a cellular receptor binding moiety.

In some embodiments, product agents are MATE agents. In some embodiments, an antibody agent moiety comprises IgG Fc region. In some embodiments, an antibody moiety is connected to a cellular receptor binding moiety through an amino group optionally through a linker. In some embodiments, it is through a lysine residue wherein the amino group of the side chain is connected to a cellular receptor binding moiety optionally through a linker (e.g., forming —NH—C(O)— as part of an amide group, a carbamate group, etc.).

In some embodiments, selected locations of antibody moieties are utilized for conjugation. For example, in some embodiments, K246 or K248 of an antibody agent (EU numbering, or corresponding residues) are conjugation locations. In some embodiments, a conjugation location is K246 of heavy chain (unless otherwise specified, locations herein include corresponding residues in, e.g., modified sequence (e.g., longer, shorter, rearranged, etc., sequences)). In some embodiments, a location is K248 of heavy chain. In some embodiments, a location is K288 or K290 of heavy chain. In some embodiments, a location is K288 of heavy chain. In some embodiments, a location is K290 of heavy chain. In some embodiments, a location is K317. In some embodiments, an antibody moiety is a moiety of an IgG1 antibody or a fragment thereof. In some embodiments, an antibody moiety is a moiety of an IgG2 antibody or a fragment thereof. In some embodiments, an antibody moiety is a moiety of an IgG4 antibody or a fragment thereof. In some embodiments, a composition comprises a plurality of MATE agents, wherein antibody moieties of the plurality of MATE agents are independently an antibody moiety of an IgG1, IgG2, or IgG4 antibody, or a fragment thereof.

In some embodiments, antibody heavy chains are selectively conjugated/labeled over light chains.

Among other things, the present disclosure can provide controlled cellular receptor binding moiety (e.g., a cellular receptor binding moiety)/antibody moiety ratios (e.g., for cellular receptor binding moiety being peptide cellular receptor binding moiety, peptide cellular receptor binding moiety/antibody ratio (PAR)). In some embodiments, a ratio is about 0.1-10, 0.5-6, etc., e.g., about 0.1, 0.2, 0.3, 0.4, 0.5, 0.6, 0.7, 0.8, or 0.9 to about 1, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, 6, 7, 8, 9, 10, about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, 6, 7, 8, 9, 10, etc.). In some embodiments, a ratio is of moieties of interest conjugated to antibody moiety and antibody moieties conjugated to moieties of interest (e.g., when a ratio is in the context of a ratio of an agent). In some embodiments, a ratio is of moieties of interest conjugated to antibody moieties and all antibodies in a composition (e.g., when a ratio is in the context of a ratio of a composition). In some embodiments, a ratio is about 0.1-6. In some embodiments, a ratio is about 0.5-2.5. In some embodiments, a ratio is about 0.5-2. In some embodiments, a ratio is about 1-2. In some embodiments, a ratio is about 1.5-2. In some embodiments, a ratio is about 1.5-2 for IgG1, IgG2 and/or IgG4 antibodies or fragments thereof. In some embodiments, for a composition, e.g., target binding moieties conjugated to IVIG, a ratio is about 1.5-2.5. In some embodiments, a ratio is about 0.1. In some embodiments, a ratio is about 0.2. In some embodiments, a ratio is about 0.3. In some embodiments, a ratio is about 0.4. In some embodiments, a ratio is about 0.5. In some embodiments, a ratio is about 0.6. In some embodiments, a ratio is about 0.7. In some embodiments, a ratio is about 0.8. In some embodiments, a ratio is about 0.9. In some embodiments, a ratio is about 1. In some embodiments, a ratio is about 1.1. In some embodiments, a ratio is about 1.2. In some embodiments, a ratio is about 1.3. In some embodiments, a ratio is about 1.4. In some embodiments, a ratio is about 1.5. In some embodiments, a ratio is about 1.6. In some embodiments, a ratio is about 1.7. In some embodiments, a ratio is about 1.8. In some embodiments, a ratio is about 1.9. In some embodiments, a ratio is about 2. In some embodiments, a ratio is about 2.1. In some embodiments, a ratio is about 2.2. In some embodiments, a ratio is about 2.3. In some embodiments, a ratio is about 2.4. In some embodiments, a ratio is about 2.5. In some embodiments, a ratio is about 1.8 for a composition wherein antibody moieties of a plurality of agents are those of an IVIG preparation. In some embodiments, a ratio of target binding moieties and antibody moieties is about 1.5-2 wherein antibody moieties of a plurality of agents are those of IgG1. In some embodiments, a ratio of target binding moieties and antibody moieties is about 1.5-2 wherein antibody moieties of a plurality of agents are those of IgG2. In some embodiments, a ratio of target binding moieties and antibody moieties is about 1.5-2 wherein antibody moieties of a plurality of agents are those of IgG4. In some embodiments, a ratio is about 1.9-2.

In some embodiments, in provided agents (e.g., agents of formula M-I or M-II, or a salt thereof) substantially all conjugation sites of antibody moieties have the same modifications (e.g., all share the same moieties of interest optionally connected through the same linker moieties). In some embodiments, no conjugation sites bear different modifications (e.g., different moieties of interest and/or no moieties of interest and/or different linker moieties).

In some embodiments, about 10%-100% of all, or substantially all, moieties of interest, e.g., target binding moieties, conjugated to antibody moieties of a particular type of antibodies (e.g., IgG1) or fragments thereof are conjugated to one or more particularly sites, typically one or two particularly sites (e.g., K246 and K248 of an IgG1 heavy chain and amino acid residues corresponding thereto). In some embodiments, about 10%-100% of all, or substantially all, moieties of interest, e.g., target binding moieties, conjugated to antibody moieties of IgG2 antibodies or fragments thereof are at K251 and K253 of an IgG2 heavy chain and amino acid residues corresponding thereto. In some embodiments, about 10%-100% of all, or substantially all, moieties of interest, e.g., target binding moieties, conjugated to antibody moieties of IgG2 antibodies or fragments thereof are at K239 and K241 of an IgG4 heavy chain and amino acid residues corresponding thereto. In some embodiments, about 10%-100% of all, or substantially all, moieties of interest (e.g., for a plurality of agents, for a composition, etc.) are conjugated to antibody moieties of IgG1, IgG2, and/or IgG4 antibodies, or fragments thereof (e.g., for conjugation products with IgG1 antibodies or fragments thereof (antibody moieties being of IgG1 antibodies or fragments thereof), IgG2 antibodies or fragments thereof (antibody moieties being of IgG2 antibodies or fragments thereof), IgG4 antibodies or fragments thereof (antibody moieties being of IgG4 antibodies or fragments thereof), or for conjugation products with IVIG (when certain provided technologies described herein are utilized, selective conjugation with IgG1, IgG2 and IgG4). In some embodiments, a percentage is about 10% or more. In some embodiments, a percentage is about 20% or more. In some embodiments, a percentage is about 25% or more. In some embodiments, a percentage is about 30% or more. In some embodiments, a percentage is about 40% or more. In some embodiments, a percentage is about 50% or more. In some embodiments, a percentage is about 60% or more. In some embodiments, a percentage is about 65% or more. In some embodiments, a percentage is about 70% or more. In some embodiments, a percentage is about 75% or more. In some embodiments, a percentage is about 80% or more. In some embodiments, a percentage is about 85% or more. In some embodiments, a percentage is about 90% or more. In some embodiments, a percentage is about 95% or more. In some embodiments, a percentage is about 100%.

In some embodiments, a composition comprises a plurality of agents (e.g., MATE agents, agents of formula M-I or M-II, or a salt thereof), each independent comprising a cellular receptor binding moiety, an antibody moiety, and optionally a linker moiety linking a cellular receptor binding moiety and an antibody moiety. In some embodiments, substantially all target binding moieties of a plurality of agents are the same. In some embodiments, substantially all target binding moieties of a plurality of agents comprise peptide moieties of a common amino acid sequence. In some embodiments, substantially all target binding moieties of a plurality of agents are peptide moieties of a common amino acid sequence. In some embodiments, substantially all conjugation sites of antibody moieties in a plurality of agents have the same modifications (e.g., all share the same moieties of interest optionally connected through the same linker moieties). In some embodiments, no conjugation sites of a plurality of agents bear different modifications (e.g., different moieties of interest and/or no moieties of interest and/or different linker moieties). In some embodiments, a plurality of agents do not contain agents that share the same (or substantially the same) antibody moieties but different modifications (e.g., different moieties of interest and/or no moieties of interest and/or different linker moieties). In some embodiments, agents that share the same (or substantially the same) antibody moieties but different modifications (e.g., different moieties of interest and/or no moieties of interest and/or different linker moieties) are intermediates of multiple-step preparations (e.g., comprising steps for removal of antibody binding moieties in addition to steps for cellular receptor binding moiety conjugation) of final product agents.

In some embodiments, the present disclosure provides a composition comprising a plurality of agents each of which independently comprising:
an antibody moiety,
a cellular receptor binding moiety, and
optionally a linker moiety linking the antibody moiety and the cellular receptor binding moiety; wherein antibody moieties of agents of the plurality comprise a common amino acid sequence, and agents of the plurality share a common cellular receptor binding moiety independently at at least one common amino acid residue of the common amino acid sequence; and wherein about 1%-100% of all agents that comprise an antibody moiety that comprise the common amino acid sequence and the cellular receptor binding moiety are agents of the plurality.

In some embodiments, the present disclosure provides a composition comprising a plurality of agents each of which independently comprising:
an antibody moiety,
a cellular receptor binding moiety, and
optionally a linker moiety linking an antibody moiety and a cellular receptor binding moiety; wherein agents of the plurality share the same or substantially the same antibody moiety, and a cellular receptor binding moiety at at least one common location; and
wherein about 1%-100% of all agents that comprise the antibody moiety and the cellular receptor binding moiety are agents of the plurality.

In some embodiments, an antibody moiety is a moiety of an IgG1 antibody or a fragment thereof. In some embodiments, an antibody moiety is a moiety of an IgG2 antibody or a fragment thereof. In some embodiments, an antibody moiety is a moiety of an IgG3 antibody or a fragment thereof. In some embodiments, an antibody moiety is a moiety of an IgG4 antibody or a fragment thereof. In some embodiments, about 1-100% of all moieties of interest are at common location(s). In some embodiments, a cellular receptor binding moiety is a cellular receptor binding moiety as described herein. In some embodiments, agents of a plurality are each independently of formula M-I or M-II, or a salt thereof.

In some embodiments, antibody moieties of agents of a plurality comprise a common amino acid sequence. In some embodiments, antibody moieties of agents of a plurality comprise a common amino acid sequence in a Fc region. In some embodiments, antibody moieties of agents of a plurality comprise a common Fc region. In some embodiments, antibody moieties of agents of a plurality can bind a common antigen specifically. In some embodiments, antibody moieties are monoclonal antibody moieties. In some embodiments, antibody moieties are polyclonal antibody moieties. In some embodiments, antibody moieties bind to two or more different antigens. In some embodiments, antibody moieties bind to two or more different proteins. In some embodiments, antibody moieties are IVIG moieties.

In some embodiments, a cellular receptor binding moiety in an agent of a plurality is a cellular receptor binding moiety. In some embodiments, each cellular receptor binding moiety is independently a cellular receptor binding moiety. In some embodiments, a composition comprises a plurality of agents, antibody moieties of agents of the plurality comprise a common amino acid sequence, and agents of a plurality share a common cellular receptor binding moiety independently linked to a common amino acid residue in the common amino acid sequence, each independently and optionally through a linker; and wherein about 1%-100% of all agents that comprise an antibody moiety that comprises a common amino acid sequence and a common cellular receptor binding moiety independently comprise a common cellular receptor binding moiety linked to a common amino acid residue independently and optionally through a linker. In some embodiments, a composition comprises a plurality of agents, antibody moieties of agents of a plurality comprise a common amino acid sequence, and agents of a plurality share a common cellular receptor binding moiety independently linked to a common amino acid residue in the common amino acid sequence, each independently through a common linker; and wherein about 1%-100% of all agents that comprise an antibody moiety that comprises a common amino acid sequence and a common cellular receptor binding moiety independently comprise a common cellular receptor binding moiety linked to a common amino acid residue independently and through a common linker. In some embodiments, a composition comprises a plurality of agents, antibody moieties of agents of a plurality comprise a common amino acid sequence, and agents of a plurality share a common cellular receptor binding moiety independently linked to a common amino acid residue in the common amino acid sequence, each independently and optionally through a linker; and wherein about 1%-100% of all agents that comprise an antibody moiety that comprises a common amino acid sequence and a common cellular receptor binding moiety are agents of a plurality. In some embodiments, a composition comprises a plurality of agents, wherein antibody moieties of agents of a plurality comprise a common amino acid sequence, and agents of a plurality share a common cellular receptor binding moiety independently linked to a common amino acid residue in the common amino acid sequence, each independently through a common linker; and wherein about 1%-100% of all agents that comprise an antibody moiety that comprises a common amino acid sequence, a common cellular receptor binding moiety, and a common linker are agents of a plurality.

As used in the present disclosure, in some embodiments, “at least one” or “one or more” is 1-1000, 1-500, 1-200, 1-100, 1-90, 1-80, 1-70, 1-60, 1-50, 1-40, 1-30, 1-20, 1-10, 1-5, or about 1, 2, 3, 4, 5, 6, 7, 8, 9, 10, 11, 12, 13, 14, 15, 16, 17, 18, 19, 20, or more. In some embodiments, it is one. In some embodiments, it is two or more. In some embodiments, it is about 3. In some embodiments, it is about 4. In some embodiments, it is about 5. In some embodiments, it is about 6. In some embodiments, it is about 7. In some embodiments, it is about 8. In some embodiments, it is about 9. In some embodiments, it is about 10. In some embodiments, it is about 10 or more.

In some embodiments, a common amino acid sequence comprises 1-1000, 1-500, 1-400, 1-300, 1-200, 1-100, 1-50, 10-1000, 10-500, 10-400, 10-300, 10-200, 10-100, 10-50, 20-1000, 20-500, 20-400, 20-300, 20-200, 20-100, 20-50, 50-1000, 50-500, 50-400, 50-300, 50-200, 50-100, 1, 2, 3, 4, 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 200, 250, 300, 400, 500, 600 or more amino acid residues. In some embodiments, a length is at least 5 amino acid residues. In some embodiments, a length is at least 10 amino acid residues. In some embodiments, a length is at least 50 amino acid residues. In some embodiments, a length is at least 100 amino acid residues. In some embodiments, a length is at least 150 amino acid residues. In some embodiments, a length is at least 200 amino acid residues. In some embodiments, a length is at least 300 amino acid residues. In some embodiments, a length is at least 400 amino acid residues. In some embodiments, a length is at least 500 amino acid residues. In some embodiments, a length is at least 600 amino acid residues.

In some embodiments, a common amino acid sequence is at least 10%-100%, 50%-100%, 50%, 55%, 60%, 65%, 70%, 75%, 80%, 85%, 90%, 91%, 92%, 93%, 94%, 95%, 96%, 97%, 98% or 99% of an amino acid sequence of an antibody moiety, a protein agent moiety, etc. In some embodiments, it is 10% or more. In some embodiments, it is 20% or more. In some embodiments, it is 30% or more. In some embodiments, it is 40% or more. In some embodiments, it is 50% or more. In some embodiments, it is 60% or more. In some embodiments, it is 70% or more. In some embodiments, it is 80% or more. In some embodiments, it is 90% or more. In some embodiments, it is 100%.

In some embodiments, in a common amino acid sequence, one and only one amino acid residue is linked to a common cellular receptor binding moiety, e.g., a common cellular receptor binding moiety. In some embodiments, in a common amino acid sequence, two and only two amino acid residues are linked to a common cellular receptor binding moiety, e.g., a common cellular receptor binding moiety. In some embodiments, in a common amino acid sequence, two or more amino acid residues are linked to a common cellular receptor binding moiety, e.g., a common cellular receptor binding moiety. In some embodiments, each common cellular receptor binding moiety, e.g., a common cellular receptor binding moiety, is independently linked to an amino acid residue in a common amino acid sequence.

In some embodiments, a common amino acid sequence comprises one or more amino acid residues selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto. In some embodiments, a common amino acid sequence comprises one or more amino acid residues selected from K246 and K248 of an IgG1 heavy chain and amino acid residues corresponding thereto. In some embodiments, a common amino acid sequence comprises one or more amino acid residues selected from K251 and K253 of an IgG2 heavy chain and amino acid residues corresponding thereto. In some embodiments, a common amino acid sequence comprises one or more amino acid residues selected from K239 and K241 of an IgG4 heavy chain and amino acid residues corresponding thereto. In some embodiments, a cellular receptor binding moiety is connected to such an amino acid residue (unless explicitly noted, optionally through a linker moiety). In some embodiments, each cellular receptor binding moiety is connected to such an amino acid residue each optionally and independently through a linker moiety.

In some embodiments, antibody moieties share a high percentage of amino acid sequence homology. In some embodiments, it is about 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, a percentage used herein, e.g., about 1%-100%, is about 10% or more. In some embodiments, a percentage is about 20% or more. In some embodiments, a percentage is about 25% or more. In some embodiments, a percentage is about 30% or more. In some embodiments, a percentage is about 40% or more. In some embodiments, a percentage is about 50% or more. In some embodiments, a percentage is about 60% or more. In some embodiments, a percentage is about 65% or more. In some embodiments, a percentage is about 70% or more. In some embodiments, a percentage is about 75% or more. In some embodiments, a percentage is about 80% or more. In some embodiments, a percentage is about 85% or more. In some embodiments, a percentage is about 90% or more. In some embodiments, a percentage is about 95% or more. In some embodiments, a percentage is about 100%.

In some embodiments, antibody moiety of agents of a plurality comprise a common Fc region or a fragment thereof.

In some embodiments, moieties of interest of agents of a plurality are at particular locations. In some embodiments, all moieties of interest are at amino acid residues of a common amino acid sequence. In some embodiments, all moieties of interest are at common locations of amino acid residues of a common amino acid sequence. In some embodiments, the number of common locations is 1. In some embodiments, it is 2. In some embodiments, it is 3. In some embodiments, it is 4. In some embodiments, antibody moieties comprise two heavy chains or fragments thereof, and the number of common locations is 2 (one on each chain). In some embodiments, common locations are selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto.

In some embodiments, agents of a plurality share a common cellular receptor binding moiety independently at at least one location. In some embodiments, agents of a plurality share a common cellular receptor binding moiety and linker independently at at least one location. In some embodiments, moieties of interest at two or more or all locations comprise a common cellular receptor binding moiety. In some embodiments, moieties of interest are the same.

In some embodiments, agents share a common modification at least one common amino acid residue. In some embodiments, agents of a plurality share a common modification at each location which is connected to a cellular receptor binding moiety and optionally a linker. In some embodiments, agents of a plurality the same -L^{PM}-TBT at each location that is connected to a linker moiety.

In some embodiments, a location is selected from K246, K248, K288, K290, K317 of antibody agents and locations corresponding thereto. In some embodiments, a location is selected from K246 and K248, and locations corresponding thereto. In some embodiments, a location is selected from K288 and K290, and locations corresponding thereto. In some embodiments, a location is K246 or a location corresponding thereto. In some embodiments, a location is K248 or a location corresponding thereto. In some embodiments, a location is K288 or a location corresponding thereto. In some embodiments, a location is K290 or a location corresponding thereto. In some embodiments, a location is K317 or a location corresponding thereto. In some embodiments, a location is K185 of light chain or a location corresponding thereto. In some embodiments, a location is K187 of light chain or a location corresponding thereto. In some embodiments, a location is K133 of heavy chain or a location corresponding thereto. In some embodiments, a location is K246 or K248 of heavy chain or a location corresponding thereto. In some embodiments, a location is K414 of heavy chain or a location corresponding thereto. In some embodiments, a common sequence is a sequence that is about or at least about 10-100, 20-50, e.g., about or at least about 10, 20, 30, 40, 50, 60, 70, 80, 90, or 100, amino acid residues in length, and comprises one or more of such residues or residues corresponding thereto. In some embodiments, a common sequence is a sequence that is about or at least about 10-100, 20-50, e.g., about or at least about 10, 20, 30, 40, 50, 60, 70, 80, 90, or 100, amino acid residues in length, and comprises one, two or more residues selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto.

In some embodiments, about 1%-100% of all agents that comprise an antibody moiety and a cellular receptor binding moiety are agents of a plurality. In some embodiments, about 1%-100% of all agents that comprise an antibody moiety that comprises a common amino acid sequence and a cellular receptor binding moiety are agents of a plurality. In some embodiments, about 1%-100% of all agents that comprise an antibody moiety that comprise a common amino acid sequence or can bind to a common antigen and a cellular receptor binding moiety are agents of a plurality. In some embodiments, about 1%-100% of all agents that comprise an antibody moiety are agents of a plurality. In some embodiments, about 1%-100% of all agents that comprise an antibody moiety that comprise the common amino acid sequence are agents of a plurality. In some embodiments, about 1%-100% of all agents that comprise a protein agent moiety that comprise the common amino acid sequence are agents of a plurality. In some embodiments, about 1%-100% of all agents that comprise an antibody agent moiety that comprise the common amino acid sequence or can bind to the common antigen are agents of a plurality. In some embodiments, a percentage is about 5%-100%. In some embodiments, a percentage is about 10%-100%. In some embodiments, a percentage is about 20%-100%. In some embodiments, a percentage is about 25%-100%. In some embodiments, a percentage is about 30%-100%. In some embodiments, a percentage is about 40%-100%. In some embodiments, a percentage is about 50%-100%. In some embodiments, it is about 5%. In some embodiments, it is about 10%. In some embodiments, it is about 20%. In some embodiments, it is about 25%. In some embodiments, it is about 30%. In some embodiments, it is about 40%. In some embodiments, it is about 50%. In some embodiments, it is about 60%. In some embodiments, it is about 70%. In some embodiments, it is about 80%. In some embodiments, it is about 90%. In some embodiments, it is about 91%. In some embodiments, it is about 50%. In some embodiments, it is about 92%. In some embodiments, it is about 93%. In some embodiments, it is about 94%. In some embodiments, it is about 95%. In some embodiments, it is about 96%. In some embodiments, it is about 97%. In some embodiments, it is about 98%. In some embodiments, it is about 99%. In some embodiments, it is about 100%. In some embodiments, it is at least about 5%. In some embodiments, it is at least about 10%. In some embodiments, it is at least about 20%. In some embodiments, it is at least about 25%. In some embodiments, it is at least about 30%. In some embodiments, it is at least about 40%. In some embodiments, it is at least about 50%. In some embodiments, it is at least about 60%. In some embodiments, it is at least about 70%. In some embodiments, it is at least about 80%. In some embodiments, it is at least about 90%. In some embodiments, it is at least about 91%. In some embodiments, it is at least about 50%. In some embodiments, it is at least about 92%. In some embodiments, it is at least about 93%. In some embodiments, it is at least about 94%. In some embodiments, it is at least about 95%. In some embodiments, it is at least about 96%. In some embodiments, it is at least about 97%. In some embodiments, it is at least about 98%. In some embodiments, it is at least about 99%.

In some embodiments, each agent of the plurality does not contain —S-Cy-, wherein -Cy- is optionally substituted 5-membered monocyclic ring, does not contain —S—S— which is not formed by cysteine residues and does not contain —SH or salt form thereof that is not of a cysteine residue. In some embodiments, each agent of the plurality does not contain —S—CH₂—CH₂—. In some embodiments, each agent of the plurality does not contain a moiety that can specifically bind to an antibody agent. In some embodiments, a composition is substantially free from a moiety that can specifically bind to an antibody agent.

In some embodiments, provided agents, compounds, etc., e.g., those of formula R-I, M-I, M-II, etc. and salts thereof have high purity. In some embodiments, a percentage is about 5%-100%. In some embodiments, a percentage is about 10%-100%. In some embodiments, a percentage is about 20%-100%. In some embodiments, a percentage is about 25%-100%. In some embodiments, a percentage is about 30%-100%. In some embodiments, a percentage is about 40%-100%. In some embodiments, a percentage is about 50%-100%. In some embodiments, it is about 5%. In some embodiments, it is about 10%. In some embodiments, it is about 20%. In some embodiments, it is about 25%. In some embodiments, it is about 30%. In some embodiments, it is about 40%. In some embodiments, it is about 50%. In some embodiments, it is about 60%. In some embodiments, it is about 70%. In some embodiments, it is about 80%. In some embodiments, it is about 90%. In some embodiments, it is about 91%. In some embodiments, it is about 50%. In some embodiments, it is about 92%. In some embodiments, it is about 93%. In some embodiments, it is about 94%. In some embodiments, it is about 95%. In some embodiments, it is about 96%. In some embodiments, it is about 97%. In some embodiments, it is about 98%. In some embodiments, it is about 99%. In some embodiments, it is about 100%. In some embodiments, it is at least about 5%. In some embodiments, it is at least about 10%. In some embodiments, it is at least about 20%. In some embodiments, it is at least about 25%. In some embodiments, it is at least about 30%. In some embodiments, it is at least about 40%. In some embodiments, it is at least about 50%. In some embodiments, it is at least about 60%. In some embodiments, it is at least about 70%. In some embodiments, it is at least about 80%. In some embodiments, it is at least about 90%. In some embodiments, it is at least about 91%. In some embodiments, it is at least about 50%. In some embodiments, it is at least about 92%. In some embodiments, it is at least about 93%. In some embodiments, it is at least about 94%. In some embodiments, it is at least about 95%. In some embodiments, it is at least about 96%. In some embodiments, it is at least about 97%. In some embodiments, it is at least about 98%. In some embodiments, it is at least about 99%.

In some embodiments, the present disclosure provides product agent compositions comprising product agents (e.g., agents of formula M-I or M-II, or a salt thereof). In some embodiments, a product agent composition (e.g., a formed agent composition from certain methods) comprises a product agent comprising an antibody moiety and a cellular receptor binding moiety and optionally a linker (e.g., an agent of formula M-I or M-II, or a salt thereof), a released antibody binding moiety (e.g., a compound comprising R^{LG} or a compound comprising a released antibody binding moiety (e.g., a compound having the structure of R^{LG}-(L^{LG1})₀₋₁(L^{LG2})₀₋₁(L^{LG3})₀₋₁(L^{LG4})₀₋₁-H or a salt thereof), and a reaction partner (e.g., a compound of formula R-I or a salt thereof). In some embodiments, released antibody binding moieties may bind to antibody moieties in target agents and/or formed product agents. Various technologies are available to separate released antibody binding moieties from antibody moieties in accordance with the present disclosure, for example, in some embodiments, contacting a composition with a composition comprising glycine at certain pH. In some embodiments, each agent of a plurality is independently such a product agent.

### Reactive Group

In some embodiments, provided agents, compounds, e.g., those useful as reaction partners such as first agents, comprise reactive groups (e.g., RG). In some embodiments, reactive groups (e.g., RG) are located between antibody binding moieties (e.g., ABT) and moieties of interest (e.g., MOI), and are optionally and independently linked to antibody binding moieties and moieties of interest via linkers. In some embodiments, RG is a reaction group as described herein.

In some embodiments, reactive groups when utilized in agents that comprise no antibody binding moieties react slowly and provide low level of, in some embodiments, substantially no conjugation of moieties of interest with target agents. As demonstrated herein, combination of reactive groups with antibody binding moieties in the same agents, e.g., as in compounds of formula R-I or salts thereof, can, among other things, promote reactions between reactive groups and target agents, enhance reaction efficiency, reduce side reactions, and/or improve reaction selectivity (e.g., in terms of target sites wherein conjugation of moieties of interest with target agents occurs).

Reactive groups in agents can react with various types of groups in target agents. In some embodiments, reactive groups in agents selectively react with amino groups of target agents, e.g., —NH₂ groups on side chains of lysine residues of proteins. In some embodiments, reactive groups when utilized in agents, e.g., those of formula R-I or salts thereof, selectively react with particular sites of target agents, e.g., as shown in examples herein, one or more of K246, K248, K288, K290, K317, etc. of IgG1, K251, K 253, etc. for IgG2, K239, K241 for IgG4, etc. In some embodiments, a site is K246 or K248 of an antibody heavy chain. In some embodiments, sites are K246 and/or K248 of an antibody heavy chain. In some embodiments, a site is K246 of an antibody heavy chain. In some embodiments, a site is K248 of an antibody heavy chain. In some embodiments, a site is K288 or K290 of an antibody heavy chain. In some embodiments, a site is K288 of an antibody heavy chain. In some embodiments, a site is K290 of an antibody heavy chain. In some embodiments, a site is K317. In some embodiments, a site is K414 of an antibody heavy chain. In some embodiments, a site is K185 of an antibody light chain. In some embodiments, a site is K187 of an antibody light chain. In some embodiments, sites are K251 and/or K253 of an IgG2 heavy chain. In some embodiments, a site is K251 of an IgG2 heavy chain. In some embodiments, a site is K253 of an IgG2 heavy chain. In some embodiments, sites are K239 and/or K241 of an IgG4 heavy chain. In some embodiments, a site is K239 of an IgG4 heavy chain. In some embodiments, a site is K241 of an IgG4 heavy chain. In some embodiments, conjugation selectively occurs at one or more heavy chain sites over light chain sites. In some embodiments, for technologies without antibody binding moieties, conjugation occurs at light chain sites more than heavy chain sites.

In some embodiments, a reactive group, e.g., RG, is or comprises an ester group. In some embodiments, a reactive group, e.g., RG, is or comprises an electrophilic group, e.g., a Michael acceptor.

In some embodiments, a reactive group, e.g., RG, is or comprises -L^{RG1}-L^{RG2}-, wherein each of L^{RG1} and L^{RG2} is independently L as described herein. In some embodiments, a reactive group, e.g., RG, is or comprises -L^{LG4}-L^{RG1}-L^{RG2}-, wherein each variable is as described herein. In some embodiments, a reactive group, e.g., RG, is or comprises -L^{LG3}-L^{LG4}-L^{RG1}-L^{RG2}-, wherein each variable is as described herein. In some embodiments, a reactive group, e.g., RG, is or comprises -L^{LG2}-L^{LG3}-L^{LG4}-L^{RG1}-L^{RG2}- wherein each variable is as described herein. In some embodiments, a reactive group, e.g., RG, is or comprises -L^{LG4}-L^{RG2}-, wherein each variable is as described herein. In some embodiments, a reactive group, e.g., RG, is or comprises -L^{LG3}-L^{LG4}-L^{RG2}-, wherein each variable is as described herein. In some embodiments, a reactive group, e.g., RG, is or comprises -L^{LG2}-L^{LG3}-L^{LG4}-L^{RG2}- wherein each variable is as described herein.

In some embodiments, as described herein, L^{LG4} is —O—. In some embodiments, L^{LG4} is —N(R)—. In some embodiments, L^{LG4} is —NH—.

In some embodiments, as described herein, L^{LG3} is or comprises an optionally substituted aryl ring. In some embodiments, L^{LG3} is or comprises a phenyl ring. In some embodiments, an aryl or phenyl ring is substituted. In some embodiments, a substituent is an electron-withdrawing group as described herein, e.g., —NO₂, —F, etc.

In some embodiments, L^{RG1} is a covalent bond. In some embodiments, L^{RG1} is not a covalent bond. In some embodiments, L^{RG1} is —S(O)₂—.

In some embodiments, L^{RG2} is —C(O)—. In some embodiments, a reactive group is or comprises -L^{LG4}-C(O)—, wherein each variable is as described herein. In some embodiments, a reactive group is or comprises -L^{LG3}-L^{LG4}-C(O)—, wherein each variable is as described herein. In some embodiments, a reactive group is or comprises -L^{LG2}-L^{LG3}-L^{LG4}-C(O)—, wherein each variable is as described herein.

In some embodiments, L^{RG2} is -L^{RG3}-C(═CR^{RG1}R^{RG2})—CR^{RG3}R^{RG4}—, wherein each of R^{RG1}, R^{RG2}, R^{RG3} and R^{RG4} is independently -L-R′, and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—. In some embodiments, each of R^{RG1}, R^{RG2}, R^{RG3} and R^{RG4} is independently R′. In some embodiments, one or more of R^{RG1}, R^{RG2}, R^{RG3} and R^{RG4} is independently —H. In some embodiments, L^{RG3} is —C(O)—. In some embodiments, L^{RG3} is —C(O)O—. In some embodiments, —O—, —N(R′)—, etc. of L^{RG3} is bonded to L^{P}M.

In some embodiments, R^{RG1} is —H. In some embodiments, R^{RG3} is —H.

In some embodiments, L^{RG2} is optionally substituted -L^{RG3}-C(═CHR^{RG2})—CHR^{RG4}—, wherein each variable is as described herein.

In some embodiments, R^{RG2} and R^{RG4} are taken together with their intervening atoms to form an optionally substituted ring as described herein. In some embodiments, a formed ring is an optionally substituted 3-10 membered monocyclic or bicyclic ring having 0-5 heteroatoms. In some embodiments, a formed ring is an optionally substituted 3-10 membered cycloaliphatic ring. In some embodiments, a formed ring is an optionally substituted 3-8 membered cycloaliphatic ring. In some embodiments, a formed ring is an optionally substituted 5-8 membered cycloaliphatic ring. In some embodiments, a formed ring is an optionally substituted 5-membered cycloaliphatic ring. In some embodiments, a formed ring is an optionally substituted 6-membered cycloaliphatic ring. In some embodiments, a formed ring is an optionally substituted 7-membered cycloaliphatic ring. In some embodiments, a formed ring is substituted. In some embodiments, a formed ring is not substituted. In some embodiments, a formed ring contains no additional unsaturation in addition to the double bond in C(═CHR^{RG2}) or C(═CR^{RG1}R^{RG2}).

In some embodiments, —C(═CHR^{RG2})—CHR^{RG4} or —C(═CR^{RG1}R^{RG2})—CR^{RG3}R^{RG4} is optionally substituted

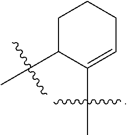 [SMILES: *C1=CCCCC1*]

In some embodiments, —C(═CHR^{RG2})—CHR^{RG4} or —C(═CR^{RG1}R^{RG2})—CR^{RG3}R^{RG4} is

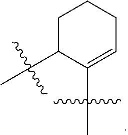 [SMILES: *C1=CCCCC1*]

In some embodiments, —[C(═CHR^{RG2})—CHR^{RG4}]-L^{RG3}- or —[C(═CR^{RG1}R^{RG2})—CR^{RG3}R^{RG4}]-L^{RG3}- is optionally substituted

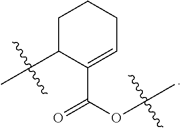 [SMILES: *OC(=O)C1=CCCCC1*]

In some embodiments, —[C(═CHR^{RG2})—CHR^{RG4}]-L^{RG3}- or —[C(═CR^{RG1}R^{RG2})—CR^{RG3}R^{RG4}]-L^{RG3}- is

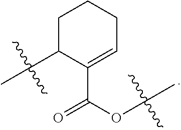 [SMILES: *OC(=O)C1=CCCCC1*]

In some embodiments, -L^{RG1}-[C(═CHR^{RG2})—CHR^{RG4}]-L^{RG3}- is optionally substituted

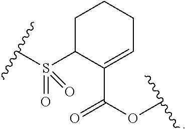 [SMILES: *OC(=O)C1=CCCCC1S(*)(=O)=O]

In some embodiments, -L^{RG1}-[C(═H^{RG2})—CH^{RG4}]-L^{RG3} or -L^{RG1}-[C═R^{RG1}R^{RG2})—C^{RG3}R^{RG4}]-L^{RG3} is optionally substituted

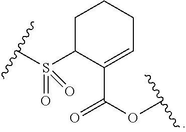 [SMILES: *OC(=O)C1=CCCCC1S(*)(=O)=O]

In some embodiments, a reactive group is a structure selected from the Table below. In some embodiments, -L^{LG2}-L^{LG3}-L^{LG4}-L^{RG1}-R^{RG2}— is a structure selected from Table below. In some embodiments, -L^{LG2}-L^{LG3}L^{LG4}-L^{RG1} is a structure selected from the Table below.

TABLE RG-1
Certain structures as examples.

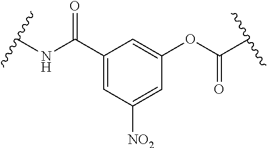 [SMILES: *NC(=O)c1cc(OC(*)=O)cc([N+](=O)[O-])c1]

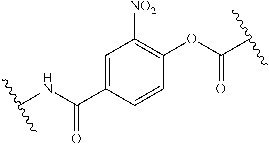 [SMILES: *NC(=O)c1ccc(OC(*)=O)c([N+](=O)[O-])c1]

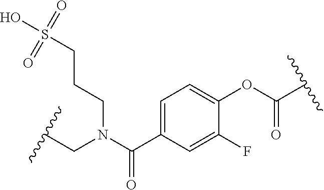 [SMILES: *CN(CCCS(=O)(=O)O)C(=O)c1ccc(OC(*)=O)c(F)c1]

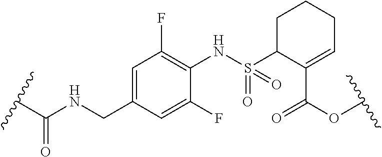 [SMILES: *OC(=O)C1=CCCCC1S(=O)(=O)Nc1c(F)cc(CNC(*)=O)cc1F]

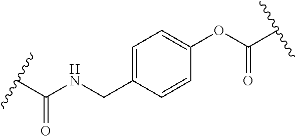 [SMILES: *C(=O)NCc1ccc(OC(*)=O)cc1]

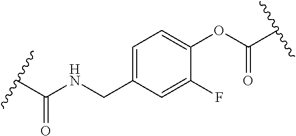 [SMILES: *C(=O)NCc1ccc(OC(*)=O)c(F)c1]

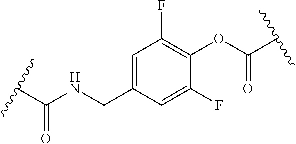 [SMILES: *C(=O)NCc1cc(F)c(OC(*)=O)c(F)c1]

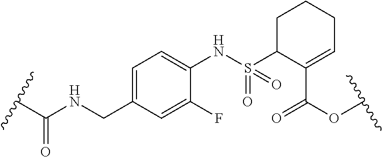 [SMILES: *OC(=O)C1=CCCCC1S(=O)(=O)Nc1ccc(CNC(*)=O)cc1F]

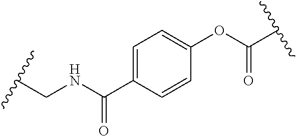 [SMILES: *CNC(=O)c1ccc(OC(*)=O)cc1]

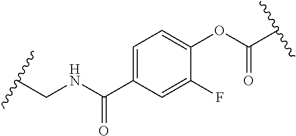 [SMILES: *CNC(=O)c1ccc(OC(*)=O)c(F)c1]

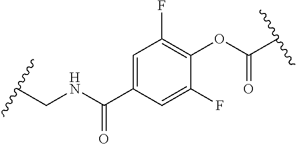 [SMILES: *CNC(=O)c1cc(F)c(OC(*)=O)c(F)c1]

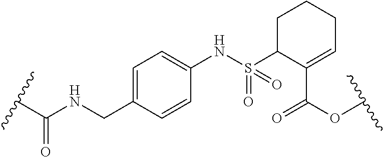 [SMILES: *OC(=O)C1=CCCCC1S(=O)(=O)Nc1ccc(CNC(*)=O)cc1]

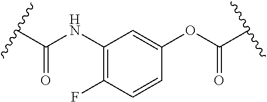 [SMILES: *C(=O)Nc1cc(OC(*)=O)ccc1F]

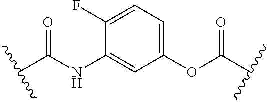 [SMILES: *C(=O)Nc1cc(OC(*)=O)ccc1F]

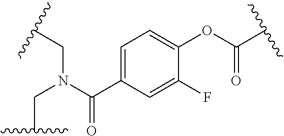 [SMILES: *CN(C*)C(=O)c1ccc(OC(*)=O)c(F)c1]

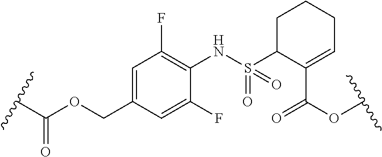 [SMILES: *OC(=O)C1=CCCCC1S(=O)(=O)Nc1c(F)cc(COC(*)=O)cc1F]

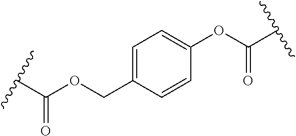 [SMILES: *C(=O)OCc1ccc(OC(*)=O)cc1]

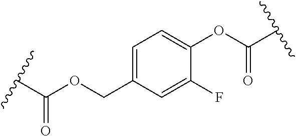 [SMILES: *C(=O)OCc1ccc(OC(*)=O)c(F)c1]

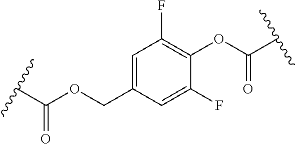 [SMILES: *C(=O)OCc1cc(F)c(OC(*)=O)c(F)c1]

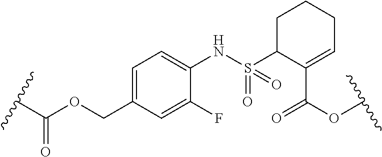 [SMILES: *OC(=O)C1=CCCCC1S(=O)(=O)Nc1ccc(COC(*)=O)cc1F]

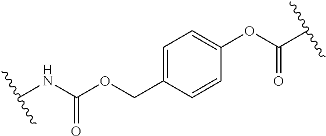 [SMILES: *NC(=O)OCc1ccc(OC(*)=O)cc1]

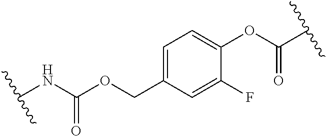 [SMILES: *NC(=O)OCc1ccc(OC(*)=O)c(F)c1]

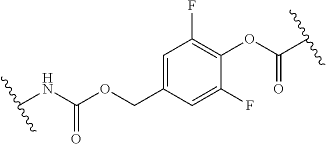 [SMILES: *NC(=O)OCc1cc(F)c(OC(*)=O)c(F)c1]

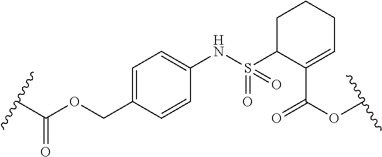 [SMILES: *OC(=O)C1=CCCCC1S(=O)(=O)Nc1ccc(COC(*)=O)cc1]

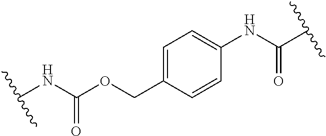 [SMILES: *NC(=O)OCc1ccc(NC(*)=O)cc1]

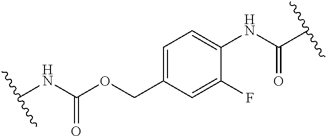 [SMILES: *NC(=O)OCc1ccc(NC(*)=O)c(F)c1]

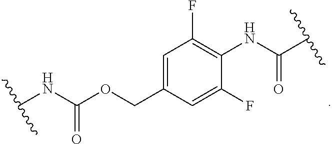 [SMILES: *NC(=O)OCc1cc(F)c(NC(*)=O)c(F)c1]

In some embodiments, -L^{LG4}-L^{RG2}- is —O—C(O). In some embodiments, -L^{LG4}-L^{RG2}- is —S—C(O)—. In some embodiments, -L^{LG4}-L^{RG1}-L^{RG2}- is —S—C(O)—.

In some embodiments, -L^{LG4}-L^{RG2}- is —N(−)—C(O)—, wherein N is a ring atom of an optionally substituted heteroaryl ring. In some embodiments, -L^{LG4}-L^{RG2}- is —N(−)—C(O)—, wherein N is a ring atom of L^{LG4} which is or comprises an optionally substituted heteroaryl ring. In some embodiments, -L^{LG4}-L^{RG2} is —N(−)—C(O)—O—, wherein N is a ring atom of L^{LG4} which is or comprises an optionally substituted heteroaryl ring.

In some embodiments, L^{RG2} is optionally substituted —CH₂—C(O)—, wherein —CH₂— is bonded to an electron-withdrawing group comprising or connected to an antibody binding moiety. In some embodiments, L^{RG2} is optionally substituted —CH₂— bonded to an electron-withdrawing group comprising or connected to an antibody binding moiety. In some embodiments, L^{RG1} is an electron-withdrawing group. In some embodiments, L^{RG1} is —C(O)—. In some embodiments, L^{RG1} is-S(O)—. In some embodiments, L^{RG1} is —S(O)₂—. In some embodiments, L^{RG1} is —P(O(OR)—. In some embodiments, L^{RG1} is —P(O(SR)—. In some embodiments, L^{RG1} is —P(O(N(R)₂)—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, L^{RG2} is optionally substituted —CH₂—C(O)—, wherein —CH₂— is bonded to a leaving group comprising or connected to an antibody binding moiety. In some embodiments, L^{RG2} is optionally substituted —CH₂— bonded to a leaving group comprising or connected to an antibody binding moiety. In some embodiments, L^{RG1} is —O—C(O)—. In some embodiments, L^{RG1} is —OS(O)₂—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, a reactive group reacts with an amino group of a target agent. In some embodiments, an amino group is —NH₂ of the side chain of a lysine residue.

In some embodiments, a target agent is a protein agent. In some embodiments, a target agent is an antibody agent. In some embodiments, a reactive group reacts with an amino acid residue of such protein or antibody agent. In some embodiments, an amino acid residue is a lysine residue. In some embodiments, a reactive group reacts with —NH₂ of the side chain of a lysine residue. In some embodiments, a reactive group is or comprises —C(O)—O—, it reacts with —NH₂ (e.g., of the side chain of a lysine residue), and forms an amide group —C(O)—O— with the —NH₂.

In some embodiments, reactive groups, e.g., a first reactive group, a second reactive group, etc., are located at terminal locations. In some embodiments, agents such as first agents comprise first reactive groups linked to target binding moieties optionally through linker moieties, and do not contain antibody binding moieties.

In some embodiments, the present disclosure provides methods for preparing a composition comprising a plurality of agents, wherein each agent independently comprises:
an antibody moiety,
a cellular receptor binding moiety, and
optionally a linker moiety linking an antibody moiety and a cellular receptor binding moiety; which method comprise:
contacting a plurality of agents each of which independently comprises a reactive group with a plurality of antibody agents.

In some embodiments, an agent comprising a reactive group comprises an antibody binding moiety, a cellular receptor binding moiety and optionally a linker. In some embodiments, agents comprising a reactive group share the same cellular receptor binding moiety. In some embodiments, agents agent comprising a reactive group share the same structure. In some embodiments, antibody molecules are of such structures, properties and/or activities to provide antibody moieties in agents described herein. In some embodiments, a plurality of antibody molecules comprise two or more IgG subclasses. In some embodiments, a plurality of antibody molecules comprise IgG1. In some embodiments, a plurality of antibody molecules comprise IgG2. In some embodiments, a plurality of antibody molecules comprise IgG4. In some embodiments, a plurality of antibody molecules comprise IgG1 and IgG2. In some embodiments, a plurality of antibody molecules comprise IgG1, IgG2 and IgG4. In some embodiments, a plurality of antibody molecules comprise IgG1, IgG2, IgG3 and IgG4. In some embodiments, a plurality of antibody molecules are IVIG antibody molecules.

In some embodiments, provided agents comprise a reactive group, e.g.,

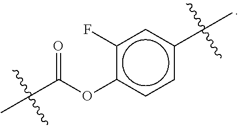

In some embodiments, —C(O)— is connected to a cellular receptor binding moiety, or a moiety comprising -(Xaa)y-, optionally through a linker and the other end is connected to an antibody binding moiety. In some embodiments,

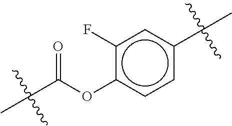

reacts with an amino group of another moiety, e.g., an antibody moiety, forming an amide group with the moiety and releasing a moiety which is or comprises antibody binding moiety. In some embodiments, an amino group is —NH₂ of a lysine side chain. In some embodiments, —C(O)— is connected to a cellular receptor binding moiety, or a moiety comprising -(Xaa)y-, optionally through a linker and the other end is connected to R′ or an optional substituent. In some embodiments, provided agents comprise optionally substituted

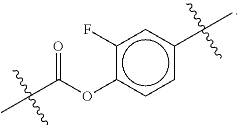

Such reactive groups may be useful for conjugation with detection, diagnosis or therapeutic agents. Those skilled in the art will appreciate that a variety of agents, and many technologies (e.g., click chemistry, reactions based on functional groups such as amino groups (e.g., amide formation), hydroxyl groups, carboxyl groups, etc.) can be utilized for conjugation in accordance with the present disclosure.

In some embodiments, antibody binding moieties bind to Fc regions of antibodies. In some embodiments, reactions occur at residues at Fc regions. In some embodiments, target binding moieties are conjugated to residues of Fc regions, optionally through linker moieties. In some embodiments, a residue is a Lys residue. In some embodiments, an antibody is or comprises IgG1. In some embodiments, an antibody is or comprises IgG2. In some embodiments, an antibody is or comprises IgG4. In some embodiments, an antibody composition utilized in a method comprises IgG1 and IgG2. In some embodiments, an antibody composition utilized in a method comprises IgG1, IgG2 and IgG4. In some embodiments, an antibody composition utilized in a method comprises IgG1, IgG2, IgG3 and IgG4.

In some embodiments, a product is or comprises IgG1. In some embodiments, a product is or comprises IgG2. In some embodiments, a product is or comprises IgG4. In some embodiments, a product composition comprises IgG1 and IgG2. In some embodiments, a product composition comprises IgG1, IgG2 and IgG4. In some embodiments, a product composition comprises IgG1, IgG2, IgG3 and IgG4.

In some embodiments, provided agents comprising antibody moieties provide one or more or substantially all antibody immune activities, e.g. for recruiting one or more types of immune cells and/or provide short-term and long-term immune activities. In some embodiments, provided agents comprising antibody moieties do not significantly reduce one or more or substantially all relevant antibody immune activities. In some embodiments, provided agents comprising antibody moieties improve one or more or substantially all relevant antibody immune activities (e.g., compared to antibody moieties by themselves). In some embodiments, provided agents provides comparable or better stability compared to antibody moieties by themselves (e.g., residence time in blood). In some embodiments, antibody moieties in provided agents can bind to FcRy of immune cells (e.g., various FcRy of immune effector cells for desired immune activities; typically at comparable or better levels). In some embodiments, antibody moieties in provided agents have comparable Fab/antigen binding capabilities. In some embodiments, antibody moieties in provided agents have comparable Fab/antigen binding capabilities. In some embodiments, antibody moieties in provided agents provide FcRn binding. In some embodiments, antibody moieties in provided agents provide FcRn binding, e.g., for antibody recycle and/or prolonged half-life. In some embodiments, provided technologies are particularly useful for modifying blood-derived IgG products as provided technologies are suitable for and can utilize all IgG subclasses.

In some embodiments, a provided method comprises one of the steps described below. In some embodiments,

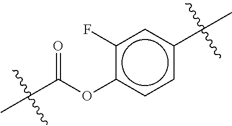

reacts with an amino group of a lysine side chain to form an amide bond with an antibody molecule, and releases

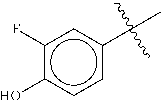

or a salt form thereof.

### Linker Moieties

In some embodiments, moieties are optionally connected to each other through linker moieties. For example, in some embodiments, a reactive group, e.g., RG, is connected to a cellular receptor binding moiety, e.g., TBT, through a linker, e.g., L^{RM}. In some embodiments, a moiety, e.g., LG, may also comprise one or more linkers, e.g., L^{LG1}, L^{LG2}, L^{LG3}, L^{LG4}, etc., to link various portions. In some embodiments, L^{LG} is a linker moiety described herein. In some embodiments, L^{LG1} is a linker moiety described herein. In some embodiments, L^{LG2} is a linker moiety described herein. In some embodiments, L^{LG3} is a linker moiety described herein. In some embodiments, L^{LG4} is a linker moiety described herein. In some embodiments, L^{RM} is a linker moiety described herein. In some embodiments, L^{PM} is L as described herein. In some embodiments, L^{PM} is a linker moiety described herein. In some embodiments, L^{PM} is L as described herein.

Linker moieties of various types and/or for various purposes, e.g., those utilized in antibody-drug conjugates, etc., may be utilized in accordance with the present disclosure.

Linker moieties can be either bivalent or polyvalent depending on how they are used. In some embodiments, a linker moiety is bivalent. In some embodiments, a linker is polyvalent and connecting more than two moieties.

In some embodiments, a linker moiety, e.g., L^{z} (wherein z represents superscript text; e.g., L^{PM}, L^{RM}, L^{LG}, L^{LG1}, etc.), is or comprises L.

In some embodiments, L is a covalent bond, or a bivalent or polyvalent optionally substituted, linear or branched C₁₋₁₀₀ group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or -[(—O—C(R′)₂—C(R′)₂—)ₙ]—, wherein n is 1-20. The linker optionally contains a cyclic group, Cy, defined below, and a reactive group, RG as defined below. In some embodiments, each amino acid residue is independently a residue of an amino acid having the structure of formula A-I or a salt thereof. In some embodiments, each amino acid residue independently has the structure of —N(R^{a1})-L^{a1}-C(R^{a2})(R^{a3})-L^{a2}-CO— or a salt form thereof.

In some embodiments, L is bivalent. In some embodiments, L is a covalent bond.

In some embodiments, L is a bivalent or optionally substituted, linear or branched group selected from C₁₋₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or -[(—O—C(R′)₂—C(R′)₂—)ₙ]—. In some embodiments, L is a bivalent or optionally substituted, linear or branched group selected from C₁₋₂₀ aliphatic and C₁₋₂₀ heteroaliphatic having 1-10 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or -[(—O—C(R′)₂—C(R′)₂—)ₙ]—. In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or -[(—O—C(R′)₂—C(R′)₂—)ₙ]—. In some embodiments, L is a bivalent or optionally substituted, linear or branched C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, an amino acid residue or -[(—O—C(R′)₂—C(R′)₂—)ₙ]—. In some embodiments, L is a bivalent or optionally substituted, linear or branched group C₁₋₁₀₀ aliphatic wherein one or more methylene units of the group are optionally and independently replaced with —C≡C—, -Cy-, —C(R′)₂—, —O—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, an amino acid residue or -[(—O—C(R′)₂—C(R′)₂—)ₙ]—. In some embodiments, L is a bivalent or optionally substituted, linear or branched group C₁₋₅₀ aliphatic wherein one or more methylene units of the group are optionally and independently replaced as described herein. In some embodiments, L is a bivalent or optionally substituted, linear or branched group C₁₋₄₀ aliphatic wherein one or more methylene units of the group are optionally and independently replaced as described herein. In some embodiments, L is a bivalent or optionally substituted, linear or branched group C₁₋₂₀ aliphatic wherein one or more methylene units of the group are optionally and independently replaced as described herein. In some embodiments, L is a bivalent or optionally substituted, linear or branched group C₁₋₁₀ aliphatic wherein one or more methylene units of the group are optionally and independently replaced as described herein. In some embodiments, L is a bivalent or optionally substituted, linear or branched group C₁₋₁₀₀ alkylene wherein one or more methylene units of the group are optionally and independently replaced as described herein. In some embodiments, L is a bivalent or optionally substituted, linear or branched group C₁₋₅₀ alkylene wherein one or more methylene units of the group are optionally and independently replaced as described herein. In some embodiments, L is a bivalent or optionally substituted, linear or branched group C₁₋₄₀ alkylene wherein one or more methylene units of the group are optionally and independently replaced as described herein. In some embodiments, L is a bivalent or optionally substituted, linear or branched group C₁₋₂₀ alkylene wherein one or more methylene units of the group are optionally and independently replaced as described herein. In some embodiments, L is a bivalent or optionally substituted, linear or branched group C₁₋₁₀ alkylene wherein one or more methylene units of the group are optionally and independently replaced as described herein.

In some embodiments, a linker moiety, e.g., L, L^{PM}, L^{RM}, etc., comprises an acidic group, e.g., —S(O)₂OH.

In some embodiments, L is or comprises -[(—O—C(R′)₂—C(R′)₂—)ₙ]—. In some embodiments, L is or comprises -[(—O—CH₂—CH₂—)ₙ]—. In some embodiments, L is -[(—CH₂—CH₂—O)₆]—CH₂—CH₂—. In some embodiments, L is -[(—CH₂—CH₂—O)₈]—CH₂—CH₂—. In some embodiments, —CH₂—CH₂—O— is bonded to an antibody binding moiety at a —CH₂—. In some embodiments, —CH₂—CH₂—O— is bonded to a cellular receptor binding moiety at a —CH₂—. In some embodiments, L^{PM} is such L as described herein. In some embodiments, L^{RM} is such L as described herein.

In some embodiments, a linker moiety, e.g., L, is or comprises, one or more —(CH₂)n-O—, wherein each n is independently 1-20. In some embodiments, it is or comprises one or more —[(CH₂)n-O]m-, wherein each n is independently 1-20, and m is 1-100. In some embodiments, it comprises two or more —[(CH₂)n-O]m-, wherein each n is independently 1-20, and each m is 1-100. In some embodiments, it is or comprises one or more —(O)C—[(CH₂)nO]m(CH₂)nNH—, —[(CH₂)nO]mNHC(O)[(CH₂)nO]mNH—, —[(CH₂)nO]m{NHC(O)[(CH₂)nO]m}pNH— wherein each n is independently 1-20, and each m is independently 1-100, and where each p is independently 1 to 10. In some embodiments, n is 1-10. In some embodiments, n is 1-5. In some embodiments, each n is 2. In some embodiments, m is 1-50. In some embodiments, m is 1-40. In some embodiments, m is 1-30. In some embodiments, m is 1-20. In some embodiments, m is 1-10. In some embodiments. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20.

In some embodiments, a linker moiety, or L, is or comprises —(CH₂CH₂O)n-, wherein each —CH₂— is independently and optionally substituted and n is 1-20. In some embodiments, a linker moiety, or L, is or comprises —(CH₂)n-O—(CH₂CH₂O)n-(CH₂)n-, wherein each n is independently 1-10, and each —CH₂— is independently and optionally substituted.

In some embodiments, a linker moiety is trivalent or polyvalent. For example, in some embodiments, a linker moiety is L as described herein and L is trivalent or polyvalent. In some embodiments, L is trivalent. For example, in some embodiments, L is —CH₂—N(—CH₂—)—C(O)—.

In some embodiments, a linker moiety, e.g., L, comprises one or more amino acid residues or analogs thereof.

In some embodiments, a linker moiety, e.g., L, L^{RM}, etc., is or comprises a reactive group as described herein. In some embodiments, an agent comprises an antibody binding moiety and a cellular receptor binding moiety linked through a linker which is or comprises a reactive group. In some embodiments, a reactive group can react with a lysine residue of an antibody in an aqueous buffer as described herein. In some embodiments, a reactive group is or comprises —C(O)—O—. In some embodiments, a reactive group is or comprises —C(O)—O—, wherein —O— is bonded to an optionally substituted aryl group. In some embodiments, a reactive group is or comprises —C(O)—O—, wherein —O— is bonded to an aryl group substituted with one or more electron-withdrawing groups. In some embodiments, one or more or each electron-withdrawing group is independently selected from —NO₂ and —F. In some embodiments, an aryl group has the structure of

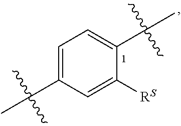

wherein R⁵ is halogen, —NO₂, —F, -L-R′, —C(O)-L-R′, —S(O)-L-R′, —S(O)₂-L-R′, or —P(O)(-L-R′)₂. In some embodiments, an aryl group has the structure of

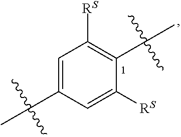

wherein each R⁵ is independently halogen, —NO₂, —F, -L-R′, —C(O)-L-R′, —S(O)-L-R′, —S(O)₂-L-R′, or —P(O)(-L-R′)₂. In some embodiments, an aryl group is

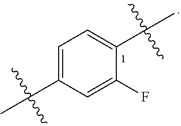 [SMILES: *c1ccc(*)c(F)c1]

In some embodiments, an aryl group is

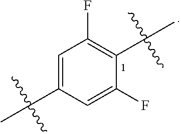 [SMILES: *c1cc(F)c(*)c(F)c1]

In some embodiments, C1 is bound to the —O— of —C(O)—O—. In some embodiments, a cellular receptor binding moiety is at the side of —C(O)— and an antibody binding moiety is at the side of —O—.

In some embodiments, a linker moiety, e.g., L, L^{RM}, etc., comprises a reactive group, wherein upon contact with an antibody, the reactive group reacts with a group of the antibody and conjugates a cellular receptor binding moiety, or a moiety comprising -(Xaa)y-, to the antibody optionally through a linker. In some embodiments, a reactive group is or comprises

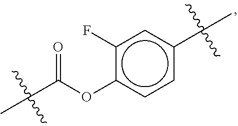

wherein the —C(O)— is connected to a cellular receptor binding moiety, or a moiety comprising -(Xaa)y-, optionally through a linker. In some embodiments, a reactive group is or comprises

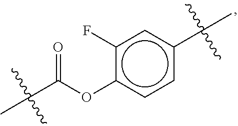

wherein the —C(O)— is connected to a cellular receptor binding moiety, or a moiety comprising -(Xaa)y-, optionally through a linker and the other end of the reactive group is connected to an antibody binding moiety.

In some embodiments, L is or comprises a bioorthogonal or enzymatic reaction product moiety. In some embodiments, L is or comprise an optionally substituted triazole moiety (which is optionally part of a bi- or poly-cyclic ring system). In some embodiments, L is or comprises LPXTG. In some embodiments, L is or comprises LPETG. In some embodiments, L is or comprises LPXT(G)n, wherein n is 1, 2, 3, 4, 5, 6, 7, 8, 9, or 10. In some embodiments, L is or comprises LPET(G)n, wherein n is 1, 2, 3, 4, 5, 6, 7, 8, 9, or 10.

In some embodiments, a linker moiety, e.g., L, L^{RM}, etc., does not contain a reactive group. In some embodiments, a linker moiety, e.g., L, L^{RM}, etc., does not contain a reactive group that readily reacts with proteins under aqueous conditions with pH about 6-9 (e.g., physiological conditions). In some embodiments, a linker moiety, e.g., L, L^{RM}, etc., does not contain a reactive group that readily reacts with natural amino acid residues under aqueous conditions with pH about 6-9 (e.g., physiological conditions).

In some embodiments, a linker moiety, e.g., L, L^{RM}, etc., comprises no —S—, wherein none of the two atoms to which the —S— is bonded to is S. In some embodiments, a linker moiety, e.g., L, L^{RM} etc., comprises no —S—S—. In some embodiments, a linker moiety, e.g., L, L^{RM}, etc., comprises no —S-bonded to a beta carbon of a carbonyl group or a double or triple bond conjugated to a carbonyl group. In some embodiments, a linker moiety, e.g., L, L^{RM}, etc., comprises no

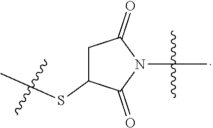 [SMILES: *SC1CC(=O)N(*)C1=O]

In some embodiments, a linker moiety, e.g., L, L^{RM}, etc., comprises no —S—.

In some embodiments, an agent comprises a linker which is not a covalent bond. In some embodiments, a linker has a length of (shortest path between linked moieties) about 1-200, 1-150, 1-100, 1-90, 1-80, 1-70, 1-60, 1-50, 1-40, 1-30, 10-200, 10-150, 10-100, 10-90, 10-80, 10-70, 10-60, 10-50, 10-40, 10-30, 20-200, 20-150, 20-100, 20-90, 20-80, 20-70, 20-60, 20-50, 20-40, 20-30, or about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 atoms or bonds.

In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not substantially damage or transform target agents and/or agents comprising target agent moieties (e.g., conjugation products comprising target agent moieties). In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not render target agents and/or agents comprising target agent moieties (e.g., conjugation products comprising target agent moieties) for one or more uses (e.g., for use as diagnostic agents, therapeutic agents, etc.). In some embodiments, provided compounds/agents (e.g., reaction partners, agents (e.g., products of provided methods and/or steps therein) comprise no cleavable groups which can be cleaved under bioorthogonal conditions. In some embodiments, provided compounds/agents (e.g., reaction partners, agents (e.g., products of provided methods and/or steps therein) comprise no cleavable groups except those which can be cleaved without substantively damaging and/or transforming proteins. In some embodiments, a cleavable group is or comprises —S—, —S—S—, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a cleavable group is or comprises —S—S—, —S—CH₂-Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a cleavage group is a cleavable linker or a cleavable portion described in US 2020/0190165, the cleavable linkers and cleavable portions of each of which is incorporated herein by reference. In some embodiments, a cleavage group is:

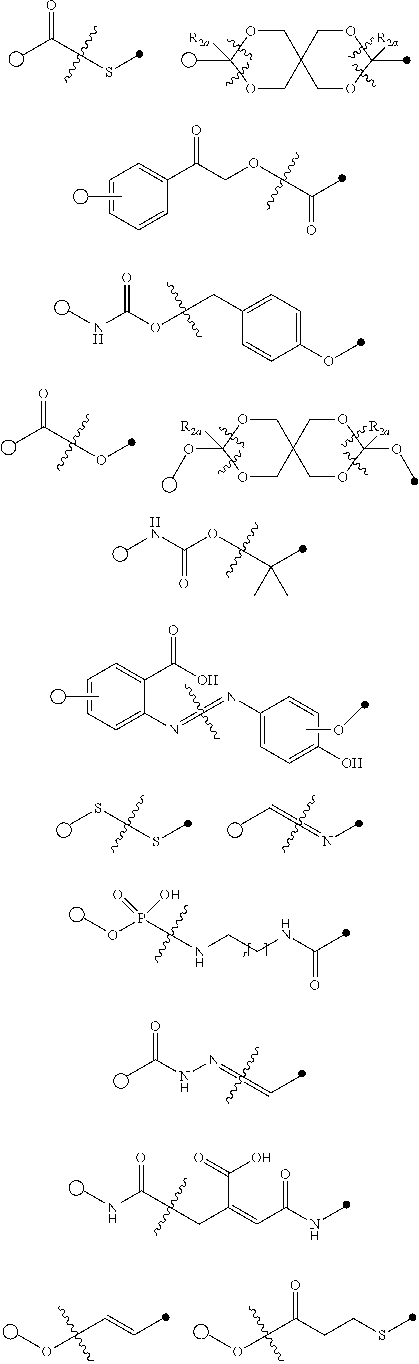

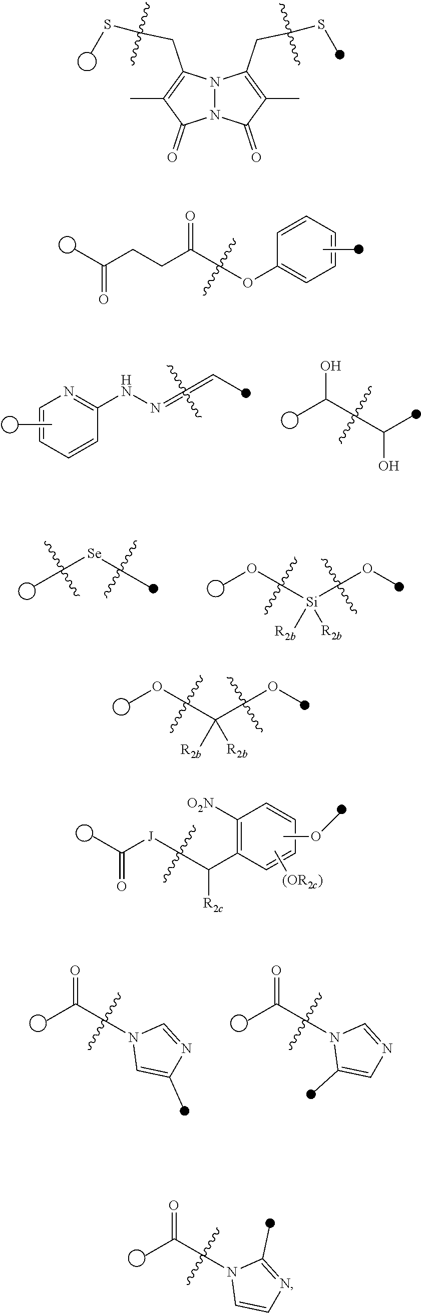

wherein:
   a wavy line orthogonal to the bond indicates a potential cleavage site,
   R^{2a}, R^{2b} and R^{2c} are the same or different and each is independently:
   (i) a hydrogen atom or a halogen atom;
   (ii) a monovalent hydrocarbon group;
   (iii) aralkyl;
   (iv) a monovalent heterocyclic group;
   (v) R_{c}—O—, R_{c}—C(O)—, R_{c}—O—C(O)—, or R_{c}—C(O)—O—, wherein R_{c} is hydrogen or a monovalent hydrocarbon group;
   (vi) —NR_{d}Rₑ, —NR_{d}Rₑ—C(O)—, —NR_{d}Rₑ—C(O)O—, —NR_{d}—C(O)—, —NR_{d}—C(O)O—, or R_{d}—C(O)—NRₑ—,
wherein R_{d} and Rₑ are the same or different and each is a hydrogen atom or a monovalent hydrocarbon group; or
   (vii) selected from a group consisting of a nitro group, a sulfuric acid group, a sulfonic acid group, a cyano group, and a carboxyl group;
   J is —CH₂—, —O—, or —S—;
   r is any integer of 1 to 4;
   white circle and black circle are independently a bond connect to other moieties.

In some embodiments, a linker moiety does not contain a cleavage group as described above. In some embodiments, a linker moiety does not contain one or more or any of the following moieties: —S—, —S—S—, —S—CH₂-Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a linker moiety does not contain one or more or any of the following moieties: —S—S—, —S—CH₂-Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a linker moiety comprises no —S—. In some embodiments, a linker moiety comprises no —S—S— (optionally except a disulfide moiety formed by two amino acid residues, in some embodiments, optionally except a disulfide moiety formed by two cysteine residues). In some embodiments, a linker moiety comprises no —S-Cy-. In some embodiments, a linker moiety comprises no —S—CH₂-Cy-. In some embodiments, a linker moiety comprises no —C(O)—O—. In some embodiments, a linker moiety comprises no —C(O)—S—. In some embodiments, a linker moiety comprises no acetal moiety. In some embodiments, a linker moiety comprises no —N═N—. In some embodiments, a linker moiety comprises no imine moiety. In some embodiments, a linker moiety comprises no —CH═N— (optionally except in a ring, in some embodiments, optionally except in a heteroaryl ring). In some embodiments, a linker moiety comprises no —P(O)(OR)O— moiety. In some embodiments, a linker moiety comprises no —P(O)(OR)—N(R)— moiety. In some embodiments, a linker moiety comprises no —C(O)—CH₂—C(COOH)═CHC(O)— moiety. In some embodiments, a linker moiety comprises no —CHOH—CHOH— moiety. In some embodiments, a linker moiety comprises no —Se— moiety. In some embodiments, a linker moiety comprises no Si bonded to two oxygen atoms. In some embodiments, a linker moiety comprises no —C(O)—CH₂—, wherein the —CH₂— is bonded to a benzylic carbon, wherein the phenyl ring of the benzyl group is substituted with —NO₂—. In some embodiments, a linker moiety comprises no —C(O)—CH₂—, wherein the —CH₂— is bonded to a benzylic carbon, wherein the phenyl ring of the benzyl group is substituted with —NO₂— at o-position. In some embodiments, a linker moiety comprise no —C(O)—N(−)— moiety, wherein N is a ring atom of a heteroaryl ring. In some embodiments, a linker moiety does not contain any of these groups. In some embodiments, L^{RM} is such a linker moiety. In some embodiments, L^{PM} is such a linker moiety. In some embodiments, L^{L}G is such a linker moiety. In some embodiments, an agent of the present disclosure does not contain one or more or all of such moieties.

In some embodiments, an agent comprises no cleavable groups whose cleavage can release LG except one or more optionally in RG. In some embodiments, an agent comprises no —S—S—, acetal or imine groups except in RG or TBT. In some embodiments, an agent comprises no —S—S—, acetal or imine groups except that the agent may have —S—S— formed by two amino acid residues. In some embodiments, an agent comprises no —S—S—, acetal or imine groups except that the agent may have —S—S— formed by cysteine residues. In some embodiments, an agent comprises no —S—S—, acetal or imine groups.

In some embodiments, L is a covalent bond. In some embodiments, L is a bivalent optionally substituted, linear or branched C₁₋₁₀₀ aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅₋₁₀₀ heteroarylaliphatic group having 1-20 heteroatoms wherein one or more methylene units of the group are optionally and independently replaced. In some embodiments, L is a bivalent optionally substituted, linear or branched C₁₋₁₀₀ heteroaliphatic group having 1-20 heteroatoms wherein one or more methylene units of the group are optionally and independently replaced.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) polyethylene glycol units. In some embodiments, a linker moiety is or comprises —(CH₂CH₂O)ₙ—, wherein n is as described in the present disclosure. In some embodiments, one or more methylene units of L are independently replaced with —(CH₂CH₂O)ₙ—.

As described herein, in some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) amino acid residues. As used in the present disclosure, “one or more” can be 1-100, 1-50, 1-40, 1-30, 1-20, 1-10, 1-5, 1, 2, 3, 4, 5, 6, 7, 8, 9, 10, 11, 12, 13, 14, 15, 16, 17, 18, 19, or 20 or more. In some embodiments, one or more methylene units of L are independently replaced with an amino acid residue. In some embodiments, one or more methylene units of L are independently replaced with an amino acid residue, wherein the amino acid residue is of an amino acid of formula A-I or a salt thereof. In some embodiments, one or more methylene units of L are independently replaced with an amino acid residue, wherein each amino acid residue independently has the structure of —N(R^{a1})-L^{a1}-C(R^{a2})(R^{a3})-L^{a2}-CO— or a salt form thereof.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′— improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety, e.g., L, comprises a —NR′— group, which can be utilized for connections with a moiety. In some embodiments, one or more methylene units of L are independently replaced with —N(R′)—.

In some embodiments, a linker moiety, e.g., L, comprises a —C(O)NR′— group, which can be utilized for connections with a moiety. In some embodiments, one or more methylene units of L are independently replaced with —C(O)N(R′)—.

In some embodiments, a linker moiety, e.g., L, comprises a —C(R′)₂— group. In some embodiments, one or more methylene units of L are independently replaced with —C(R′)₂—. In some embodiments, —C(R′)₂— is —CHR′—. In some embodiments, R′ is —(CH₂)₂C(O)NH(CH₂)nCOOH. In some embodiments, R′ is —(CH₂)₂COOH. In some embodiments, R′ is —COOH.

In some embodiments, a linker moiety is or comprises one or more ring moieties, e.g., one or more methylene units of L are replaced with -Cy-. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

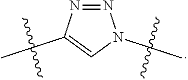 [SMILES: *c1cn(*)nn1]

In some embodiments, a methylene unit of L is replaced with

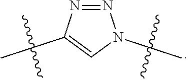 [SMILES: *c1cn(*)nn1]

In some embodiments, a methylene unit of L is replaced with -Cy-. In some embodiments, -Cy- is

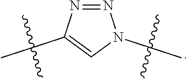 [SMILES: *c1cn(*)nn1]

In some embodiments, a linker moiety (e.g., L) is or comprises —CO)y-. In some embodiments L is or comprises —[(CH₂)nO]mCy[(CH₂)nO]mNH, or L is —[(CH₂)nO]mCy[(CH₂)nO]mNHC(O)[(CH₂)nO]mNH—, or L is —[(CH₂)nO]mCy[(CH₂)nO]m{NHC(O)[(CH₂)nO]m}pNH—, where n, m, and p are independently chosen at each occurrence from 1-20, from 1-12, or 2-10. In some embodiments each n is 2, m is independently chosen at each occurrence from an integer from 2-10, or in some embodiments m is independently chosen from an integer from 2-6 and Cy is

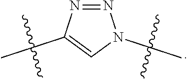 [SMILES: *c1cn(*)nn1]

In some embodiments, a methylene unit of L is replaced with -Cy-. In some embodiments, -Cy- is

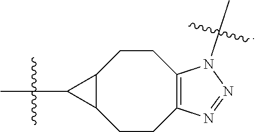 [SMILES: *C1C2CCc3nnn(*)c3CCC12]

In some embodiments, -Cy- is

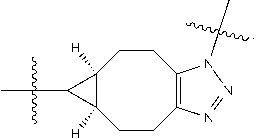 [SMILES: *C1[C@H]2CCc3c(nnn3*)CC[C@@H]12]

In some embodiments, -Cy- is

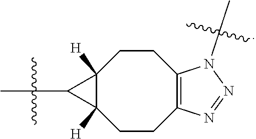 [SMILES: *C1[C@H]2CCc3nnn(*)c3CC[C@@H]12]

In some embodiments, a linker moiety, e.g., L, in a provided agent, e.g., a compound in Table 1, comprises

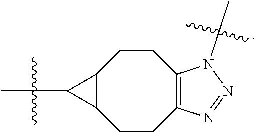 [SMILES: *C1C2CCc3nnn(*)c3CCC12]

In some embodiments,

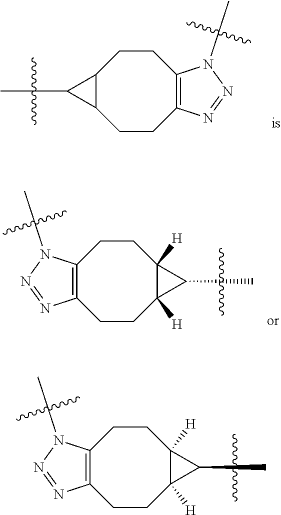

in the structure. In some embodiments

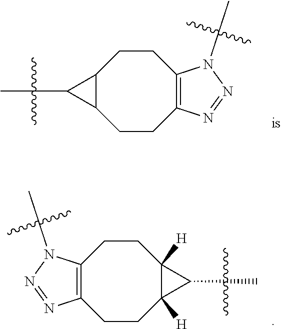

In some embodiments,

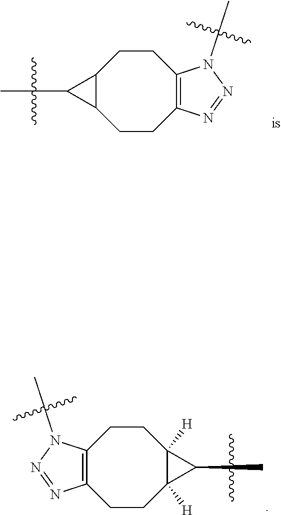

In some embodiments, a linker moiety is as described in Table 1. In some embodiments, L is L¹ as described in the present disclosure. In some embodiments, L is L^{b} as described in the present disclosure.

In some embodiments, L^{RM} is a covalent bond. In some embodiments, L^{RM} is not a covalent bond. In some embodiments, L^{RM} is or comprises —(CH₂CH₂O)ₙ—. In some embodiments, L^{RM} is or comprises —(CH₂)ₙ—O—(CH₂CH₂O)ₙ—(CH₂)ₙ—, wherein each n is independently as described herein, and each —CH₂— is independently optionally substituted. In some embodiments, L^{RM} is —(CH₂)ₙ—O—(CH₂CH₂O)ₙ—(CH₂)ₙ—, wherein each n is independently as described herein, and each —CH₂— is independently optionally substituted. In some embodiments, L^{RM} is —(CH₂)₂—O—(CH₂CH₂O)ₙ—(CH₂)₂—, wherein n is as described herein, and each —CH₂— is independently optionally substituted. In some embodiments, L^{RM} is —(CH₂)₂—O—(CH₂CH₂O)ₙ—(CH₂)₂—, wherein n is as described herein.

In some embodiments, L^{PM} is a covalent bond. In some embodiments, L^{PM} is not a covalent bond. In some embodiments, L^{PM} is or comprises —(CH₂CH₂O)ₙ—. In some embodiments, L^{PM} is or comprises —(CH₂)ₙ—O—(CH₂CH₂O)ₙ—(CH₂)ₙ—, wherein each n is independently as described herein, and each —CH₂— is independently optionally substituted. In some embodiments, L^{PM} is —(CH₂)ₙ—O—(CH₂CH₂O)ₙ—(CH₂)ₙ—, wherein each n is independently as described herein, and each —CH₂— is independently optionally substituted. In some embodiments, L^{PM} is —(CH₂)₂—O—(CH₂CH₂O)ₙ—(CH₂)₂—, wherein n is as described herein, and each —CH₂— is independently optionally substituted. In some embodiments, L^{PM} is —(CH₂)₂—O—(CH₂CH₂O)ₙ—(CH₂)₂—, wherein n is as described herein.

In some embodiments, L^{PM} (e.g., in a product of a first and a second agents) is or comprises a reaction product moiety formed a first reactive moiety and a second reactive moiety.

In some embodiments, a linker moiety (e.g., L^{PM} in a product of a first and a second agents) is or comprises

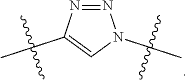 [SMILES: *c1cn(*)nn1]

In some embodiments, a methylene unit of a linker moiety, e.g., L or a linker moiety that can be L (e.g., L^{RM}, L^{PM}, etc.) is replaced with -Cy-. In some embodiments, -Cy- is optionally substituted

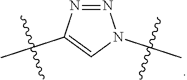 [SMILES: *c1cn(*)nn1]

In some embodiments, -Cy- is

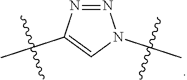 [SMILES: *c1cn(*)nn1]

In some embodiments L is —[(CH₂)nO]mCH₂Cy[(CH₂)nO]m- or ˜˜ In some embodiments, -Cy- is

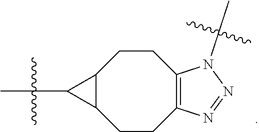

In some embodiments, -Cy- is

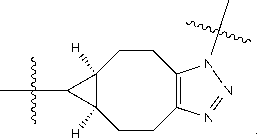

In some embodiments, -Cy- is

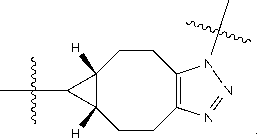

### Cellular Receptor Binding Moiety

Various receptor binding moieties, according to embodiments of present invention, are described in WO2019/199621A1 published Oct. 17, 2019, WO2019/199634 published Oct. 17, 2019, International Application No. PCT/US2020/055053 filed Oct. 9, 2020, and International Application No. PCT/US2020/055053 filed Oct. 9, 2020, each of which is incorporated herein in its entirety by reference.

In an embodiment, the cellular receptor binding moiety may include an ASGPR binding group connected through an amine group. The term “asialoglycoprotein receptor (ASGPR) binding group” refers to a binding group which binds to hepatocyte asialoglycoprotein receptor. This binding group is also a component of the presently claimed bifunctional compounds as a cellular receptor binding moiety which is covalently bound to the antibody binding moiety through a linker group or directly. The ASGPR group selectively binds to hepatocyte asialoglycoprotein receptor on the surface of hepatocytes. It is through this moiety that bifunctional compounds complexed with circulating protein (e.g., galactose-deficient IgA1) bind to hepatocytes. Once bound to the hepatocyte, the circulating protein is taken into the hepatocytes or other cells via a phagocytosis mechanism, wherein the circulating protein is degraded through lysosomal degradation.

The amine group may be a primary alkyl amine group or secondary alkyl amine group, each of which is optionally substituted on the amine group with a C₁-C₃ alkyl group.

The cellular receptor binding moiety may include an ASGPR binding group according to the chemical structure:

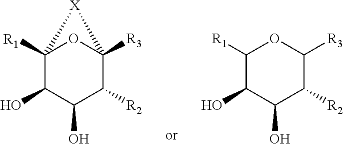

wherein X is 1-4 atoms in length and comprises O, S, N(R^{N1}) or C(R^{N1})(R^{N1}) groups such that
when X is 1 atom in length, X is O, S, N(R^{N1}) or C(R^{N1})(R^{N1}),
when X is 2 atoms in length, no more than 1 atom of X is O, S or N(R^{N1}),
when X is 3 or 4 atoms in length, no more than 2 atoms of X are 0, S or N(R^{N1});
wherein R^{N1} is H or a C₁-C₃ alkyl group optionally substituted with from 1-3 halo groups; R₁ and R₃ are each independently
   H, —(CH₂)_{K}OH, —(CH₂)_{K}OC₁-C₄ alkyl, which is optionally substituted with from 1-3 halo groups, C₁-C₄ alkyl, which is optionally substituted with from 1-3 halo groups, —(CH₂)_{K}-vinyl, O—(CH₂)_{K}-vinyl, —(CH₂)_{K}-alkynyl, —(CH₂)_{K}—COOH,
   —(CH₂)_{K}C(O)O—C₁-C₄ alkyl which is optionally substituted with from 1-3 halo groups, 0—C(O)—C₁-C₄ alkyl, which is optionally substituted with from 1-3 halo groups, —C(O)—C₁-C₄ alkyl, which is optionally substituted with from 1-3 halo groups, or

R₁ and R₃ are each independently a

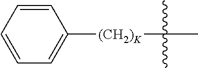

group, which is optionally substituted with up to three halo groups, C₁-C₄ alkyl groups, each of which alkyl group is optionally substituted with from one to three halo groups or one or two hydroxyl groups, or O—C₁-C₄ alkyl groups, each of which alkyl groups is optionally substituted with from one to three halo groups or one or two hydroxyl groups; and
K is independently an integer of 0 to 4, or
R₁ and R₃ are each independently a group according to the chemical structure:

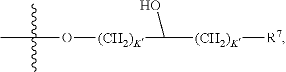

wherein R⁷ is O—C₁-C₄ alkyl, which is optionally substituted with from 1 to 3 halo groups 1 or 2 hydroxy groups, or R⁷ is a
—NR^{N3}R^{N4} group or a

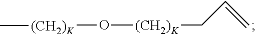

or
R₁ and R₃ are each independently a group according to the structure:

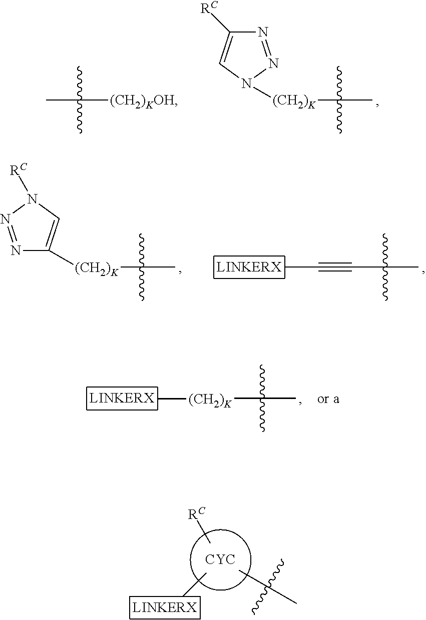

group according to the chemical structure:

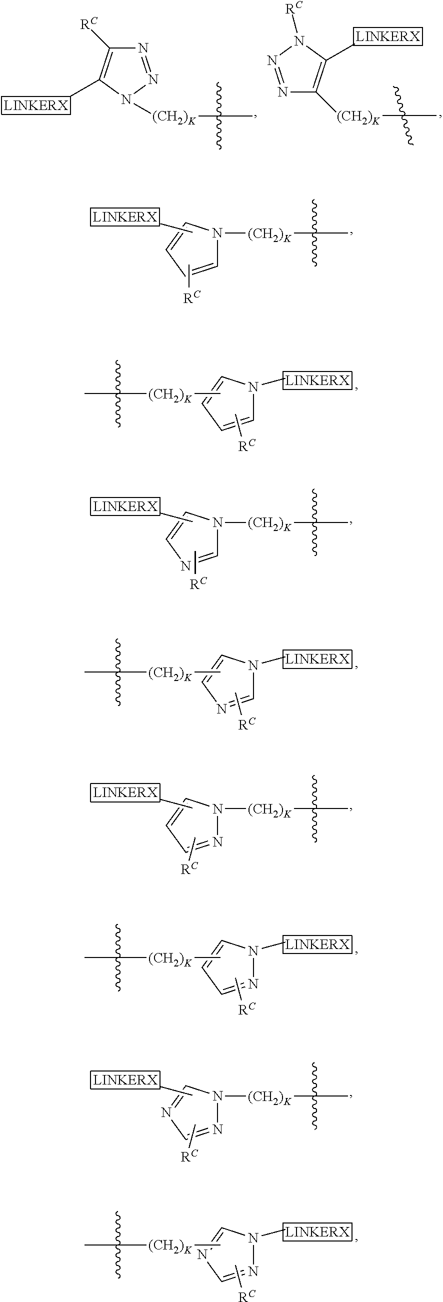

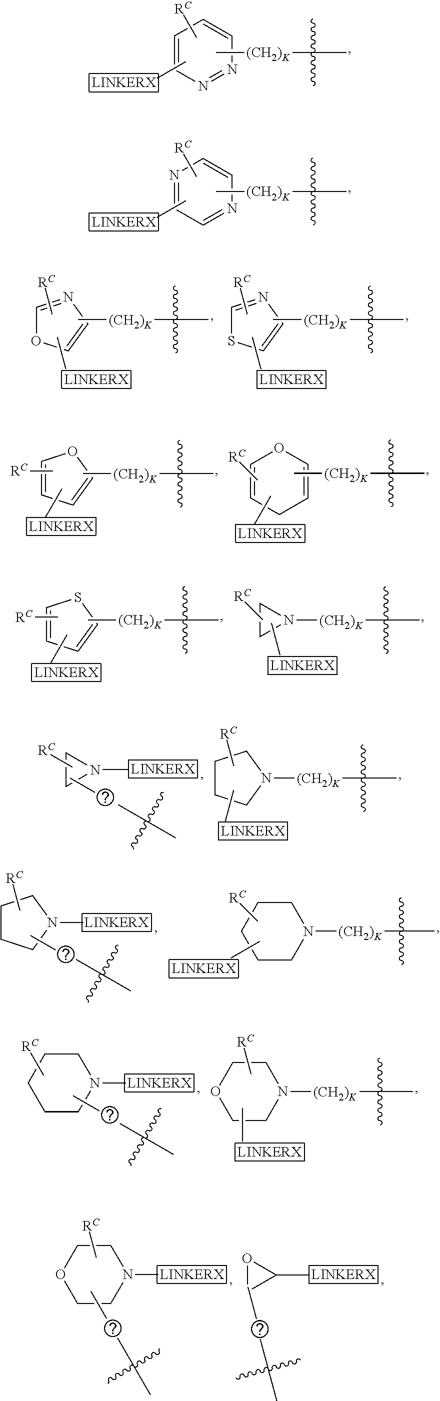

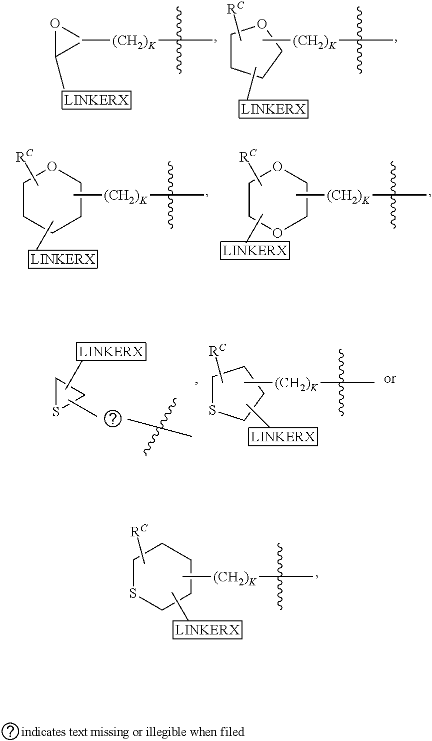

or
R₁ and R₃ are each independently a

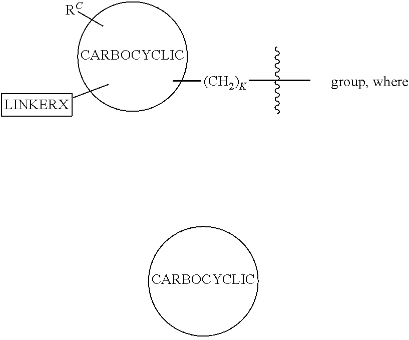

is a C₃-C₈ saturated carbocyclic group;
R^{C} is absent, H, C₁-C₄ alkyl which is optionally substituted with from 1-3 halo groups or 1-2 hydroxyl groups, or a group according to the structure:

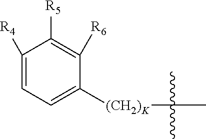

wherein R₄, R₅ and R₆ are each independently, H, halo (F, Cl, Br, I), CN, NR^{N1}R^{N2}, —(CH₂)_{K}OH,
—(CH₂)_{K}OC₁-C₄ alkyl, which is optionally substituted with from 1-3 halo groups, C₁-C₃ alkyl, which is optionally substituted with from 1-3 halo groups, —O—C₁-C₃-alkyl, which is optionally substituted with from 1-3 halo groups, —(CH₂)_{K}COOH, —(CH₂)_{K}C(O)O—C₁-C₄ alkyl which is optionally substituted with from 1-3 halo groups, O—C(O)—C₁-C₄ alkyl, which is optionally substituted with from 1-3 halo groups, —C(O)—C₁-C₄ alkyl, which is optionally substituted with from 1-3 halo groups, or

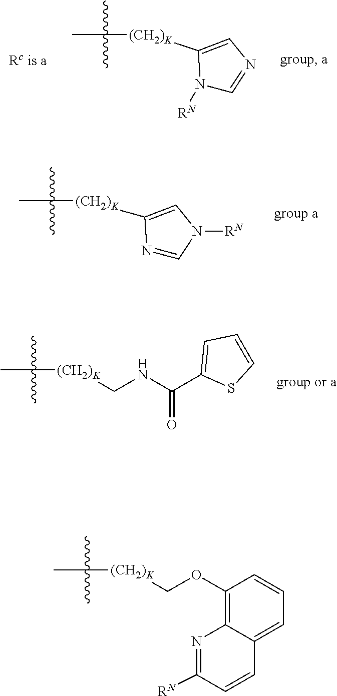

group,
wherein R^{N}, R^{N1} and R^{N2} are each independently H or a C₁-C₃ alkyl group which is optionally substituted with from one to three halo groups or one or two hydroxyl groups;
K is independently an integer of 0 to 4;
K′ is an integer of 1 to 4;
R^{N3} is H, or a C₁-C₃ alkyl group which is optionally substituted with 1-3 halo groups or 1 or 2 hydroxy groups; and
R^{N4} is H, a C₁-C₃ alkyl group which is optionally substituted with 1-3 halo groups or 1 or 2 hydroxy groups, or R^{N4} is a

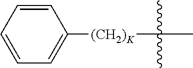

group, where K is preferably 1;
[symbol] is a linker group which comprises at least one glycan-specific IgG antibody moiety and links the at least one glycan-specific IgG antibody moiety to the cellular receptor binding moiety through the optional linker moiety, or
[symbol] is a linker group which contains at least one or more functional groups which can be used to covalently bond the linker group to at least one glycan-specific IgG antibody moiety or optional linker moiety;
R₂ is a

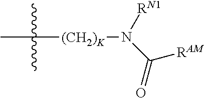

group wherein R^{N1} and K are the same as above;
R^{AM} is H, a C₁-C₄ alkyl group optionally substituted with up to 3 halo groups and one or two hydroxyl groups, a —(CH₂)_{K}COOH group, a —(CH₂)_{K}C(O)O—C₁-C₄ alkyl group which is optionally substituted with from 1-3 halo groups, a O—C(O)—C₁-C₄ alkyl group, which is optionally substituted with from 1-3 halo F groups, a —C(O)—C₁-C₄ alkyl group, which is optionally substituted with from 1-3 halo groups, a —(CH₂)_{K}—NR^{N3}R^{N4} group where R^{N3} is H, or a C₁-C₃ alkyl group which is optionally substituted with 1-3 halo groups or 1 or 2 hydroxy groups; and
R^{N4} is H, a C₁-C₃ alkyl group which is optionally substituted with 1-3 halo groups or 1 or 2 hydroxy groups, or a

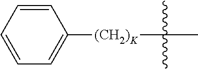

group, or
R₂ is

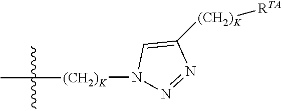

group,
wherein R^{TA} is H, CN, NR^{N1}R^{N2}, —(CH₂)_{K}OH, —(CH₂)_{K}OC₁-C₄ alkyl, which is optionally substituted with from 1-3 halo groups, C₁-C₄ alkyl, which is optionally substituted with from 1-3 halo groups, —(CH₂)_{K}COOH, —(CH₂)_{K}C(O)O—C₁-C₄ alkyl which is optionally substituted with from 1-3 halo groups, O—C(O)—C₁-C₄ alkyl, which is optionally substituted with from 1-3 halo groups, —C(O)—C₁-C₄ alkyl, which is optionally substituted with from 1-3 halo groups, or
R^{TA} is a C₃-C₁₀ aryl or a three- to ten-membered heteroaryl group containing up to 5 heteroaryl atoms, each of said aryl or heteroaryl groups being optionally substituted with up to three (preferably 1) CN, NR^{N1}R^{N2}, —(CH₂)_{K}OH, —(CH₂)_{K}OC₁-C₄ alkyl, which is optionally substituted with from 1-3 halo groups, C₁-C₃ alkyl, which is optionally substituted with from 1-3 halo groups or 1 or 2 hydroxy groups, —O—C₁-C₃-alkyl, which is optionally substituted with from 1-3 halo groups, —(CH₂)_{K}COOH, —(CH₂)_{K}C(O)O—C₁-C₄ alkyl which is optionally substituted with from 1-3 halo groups, O—C(O)—C₁-C₄ alkyl, which is optionally substituted with from 1-3 halo groups or —(CH₂)_{K}C(O)—C₁-C₄ alkyl which is optionally substituted with from 1-3 halo groups, or
R^{TA} is a

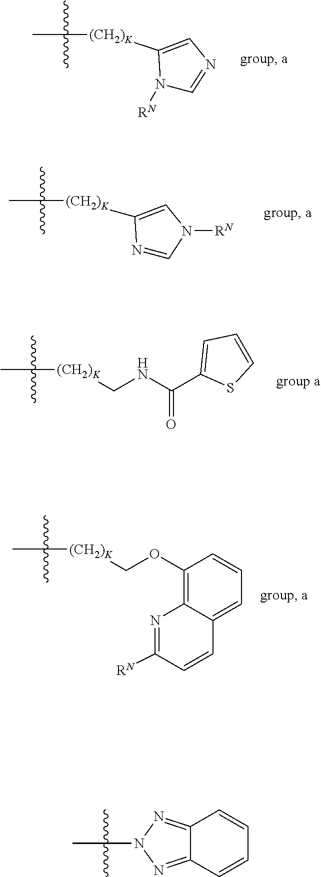

group which is optionally substituted with up to three C₁-C₃ alkyl groups which are optionally substituted with up to three halo groups, or
R^{TA} is a

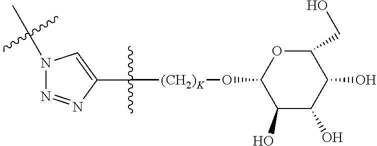

group,
wherein R^{N}, R^{N1} and R^{N2} are each independently H or a C₁-C₃ alkyl group which is optionally substituted with from one to three halo groups or one or two hydroxyl groups and each —(CH₂)_{K} group is optionally substituted with 1-4, preferably 1 or 2, C₁-C₃ alkyl groups which are optionally substituted with from 1-3 fluoro groups or 1-2 hydroxyl groups;
and K is independently 0-4.

The cellular receptor binding moiety may include an ASGPR binding group according to the chemical structure:

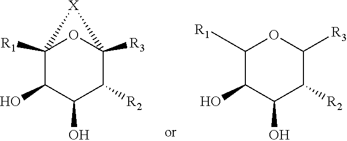

wherein X is 1-4 atoms in length and comprises O, S, N(R^{N1}) or C(R^{N1})(R^{N1}) groups such that
when X is 1 atom in length, X is O, S, N(R^{N1}) or C(R^{N1})(R^{N1}),
when X is 2 atoms in length, no more than 1 atom of X is O, S or N(R^{N1}),
when X is 3 or 4 atoms in length, no more than 2 atoms of X are 0, S or N(R^{N1});
wherein each R^{N1} is independently H or a C₁-C₃ alkyl group optionally substituted with from 1-3 halo groups, preferably F (R^{N1} is preferably H or methyl, more often H);
R₁ and R₃ are each independently H, —(CH₂)_{K}OH, —(CH₂)_{K}OC₁-C₄ alkyl, which is optionally substituted with from 1-3 halo (F, Cl, Br, I, preferably F) groups, C₁-C₄ alkyl, which is optionally substituted with from 1-3 halo (F, Cl, Br, I, preferably F) groups, —(CH₂)_{K}vinyl, O—(CH₂)_{K}Vinyl, —(CH₂)_{K}alkynyl, —(CH₂)_{K}COOH, —(CH₂)_{K}C(O)O—C₁-C₄ alkyl which is optionally substituted with from 1-3 halo, preferably F groups, O—C(O)—C₁-C₄ alkyl, which is optionally substituted with from 1-3 halo, preferably F groups, —C(O)—C₁-C₄ alkyl, which is optionally substituted with from 1-3 halo, preferably F groups, or
R₁ and R₃ are each independently a

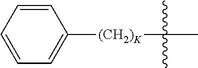

group, which is optionally substituted with up to three (preferably 1) halo groups (preferably F), C₁-C₄ alkyl groups, each of which is optionally substituted with from one to three halo groups, preferably F, or one or two hydroxyl groups, or
O—C₁-C₄ alkyl groups, each of which alkyl groups is optionally substituted with from one to three halo groups, preferably F, or one or two hydroxyl groups, and K is independently 0-4 (0, 1, 2, 3 or 4), or
R₁ and R₃ are each independently a group according to the chemical structure:

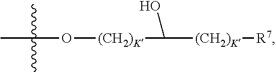

where R⁷ is O—C₁-C₄ alkyl, which is optionally substituted with from 1 to 3 halo groups, preferably F and 1 or 2 hydroxy groups, or R⁷ is a
—N^{N3}R^{N4} group or a

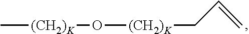

or
R₁ and R₃ are each independently a group according to the structure:

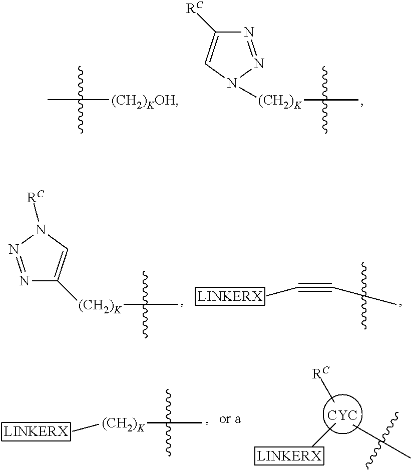

group according to the chemical structure:

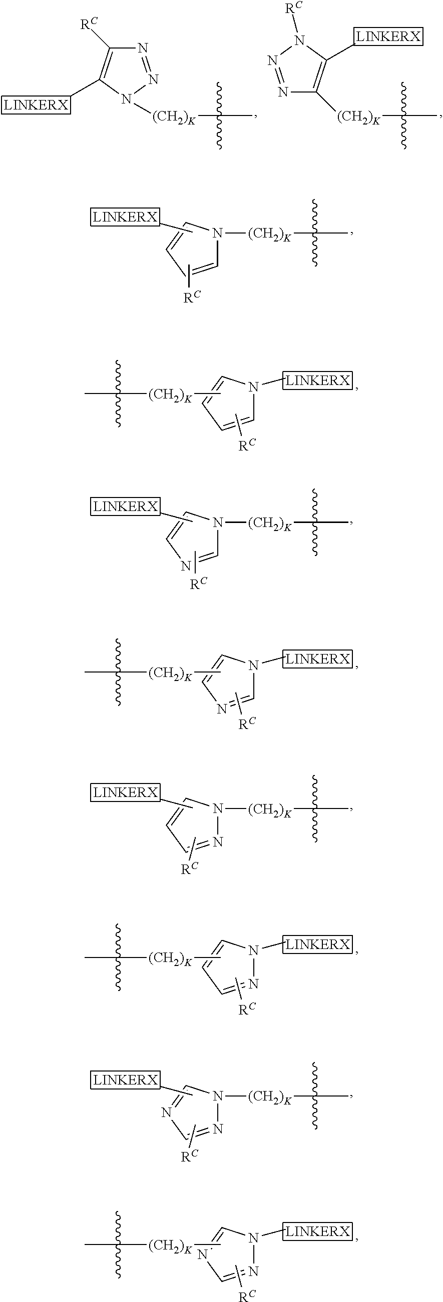

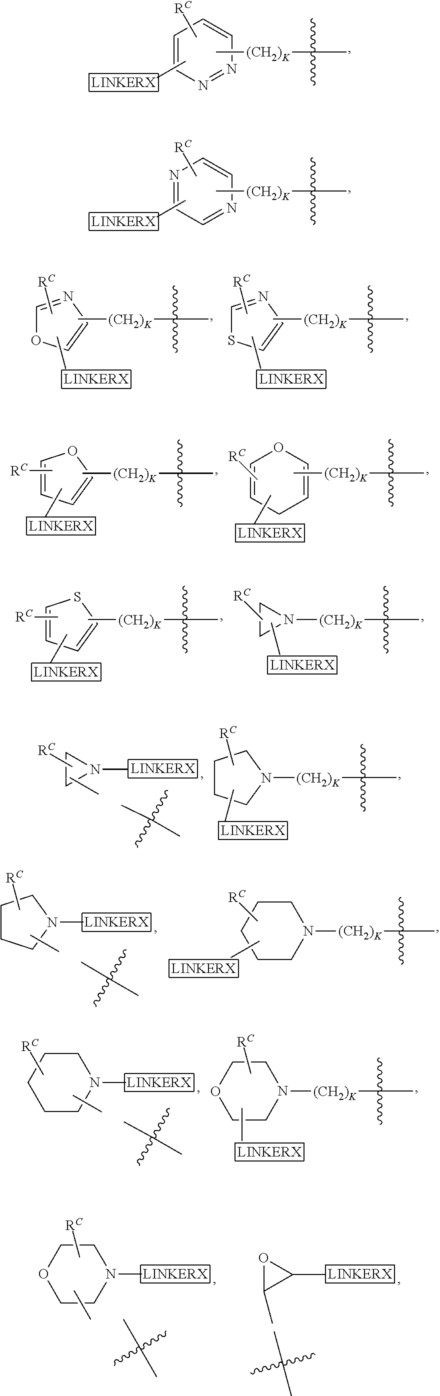

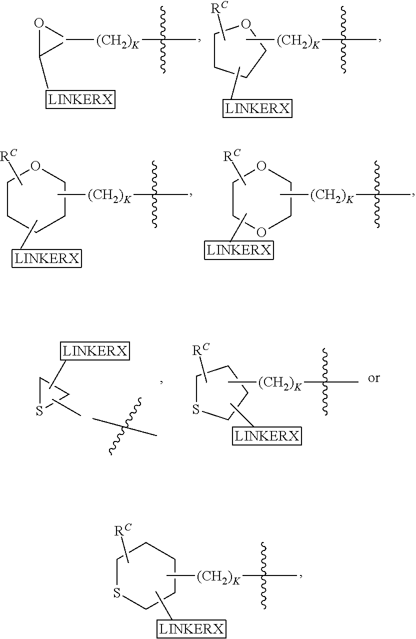

or R₁ and R₃ are each independently a

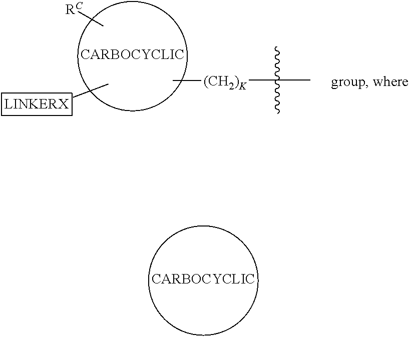

is a C₃-C₈ saturated carbocyclic group;
R^{C} is absent, H, C₁-C₄ alkyl which is optionally substituted with from 1-3 halo (preferably fluoro) groups or 1-2 hydroxyl groups, or a group according to the structure:

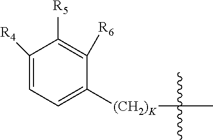

where R₄, R₅ and R₆ are each independently, H, halo (F, Cl, Br, I), CN, NR^{N1}R^{N2}
—(CH₂)_{K}OH, —(CH₂)_{K}OC₁-C₄ alkyl, which is optionally substituted with from 1-3 halo (F, Cl, Br, I, preferably F) groups, C₁-C₃ alkyl, which is optionally substituted with from 1-3 halo (F, Cl, Br, I, preferably F) groups, —O—C₁-C₃-alkyl, which is optionally substituted with from 1-3 halo, preferably F groups, —(CH₂)_{K}COOH, —(CH₂)_{K}C(O)O—C₁-C₄ alkyl which is optionally substituted with from 1-3 halo, preferably F groups, O—C(O)—C₁-C₄ alkyl, which is optionally substituted with from 1-3 halo, preferably F groups, —C(O)— C₁-C₄ alkyl, which is optionally substituted with from 1-3 halo, preferably F groups, or
R^{C} is a

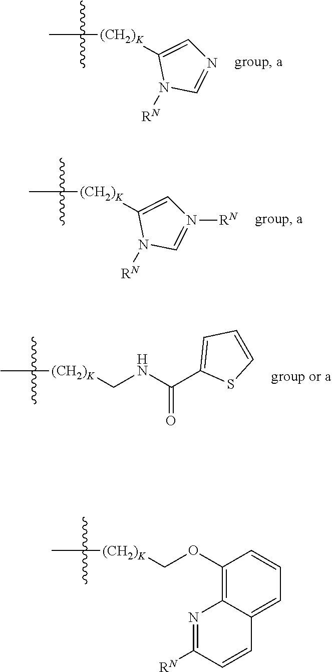

group,
where R^{N}, R^{N1} and R^{N2} are each independently H or a C₁-C₃ alkyl group which is optionally substituted with from one to three halo groups, preferably F, or one or two hydroxyl groups;
K is independently 0-4 (0, 1, 2, 3 or 4), preferably 0 or 1;
K′ is 1-4, preferably 1;
R^{N3} is H, or a C₁-C₃ alkyl group which is optionally substituted with 1-3 halo groups, preferably F or 1 or 2 hydroxy groups; and
R^{N4} is H, a C₁-C₃ alkyl group which is optionally substituted with 1-3 halo groups, preferably F or 1 or 2 hydroxy groups, or R^{N4} is a

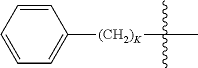

group, where K is preferably 1;
[symbol] is a linker group which comprises at least one glycan-specific IgG antibody moiety and links the at least one glycan-specific IgG antibody moiety to the cellular receptor binding moiety through the optional linker moiety, or
[symbol] is a linker group which contains at least one or more functional groups which can be used to covalently bond the linker group to at least one glycan-specific IgG antibody moiety or optional linker moiety;
R₂ is a

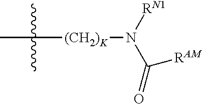

group where R^{N1} and K are the same as above;
R^{AM} is H, a C₁-C₄ alkyl group optionally substituted with up to 3 halo groups (preferably F) and one or two hydroxyl groups, a —(CH₂)_{K}COOH group, a —(CH₂)_{K}C(O)O—C₁-C₄ alkyl group which is optionally substituted with from 1-3 halo, preferably F groups,
O—C(O)—C₁-C₄ alkyl group, which is optionally substituted with from 1-3 halo, preferably F groups, a —C(O)—C₁-C₄ alkyl group, which is optionally substituted with from 1-3 halo, preferably F groups, a —(CH₂)_{K}—NR^{N3}R^{N4} group where R^{N3} is H, or a C₁-C₃ alkyl group which is optionally substituted with 1-3 halo groups, preferably F or 1 or 2 hydroxy groups, or
R₂ is a

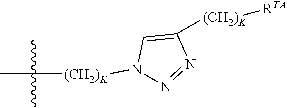

group, wherein R^{TA} is H, CN, NR^{N1}R^{N2}, —(CH₂)_{K}OH, —(CH₂)_{K}OC₁-C₄ alkyl, which is optionally substituted with from 1-3 halo (F, Cl, Br, I, preferably F) groups, C₁-C₄ alkyl, which is optionally substituted with from 1-3 halo (F, Cl, Br, I, preferably F) groups, —(CH₂)_{K}COOH, —(CH₂)_{K}C(O)O—C₁-C₄ alkyl which is optionally substituted with from 1-3 halo, preferably F groups, O—C(O)—C₁-C₄ alkyl, which is optionally substituted with from 1-3 halo, preferably F groups, —C(O)—C₁-C₄ alkyl, which is optionally substituted with from 1-3 halo, preferably F groups, or R^{TA} is a C₃-C₁₀ aryl or a three- to ten-membered heteroaryl group containing up to 5 heteroaryl atoms, each of said aryl or heteroaryl groups being optionally substituted with up to three (preferably 1) CN, NR^{N1}R^{N2}, —(CH₂)_{K}OH, —(CH₂)_{K}OC₁-C₄ alkyl, which is optionally substituted with from 1-3 halo (F, Cl, Br, I, preferably F) groups, C₁-C₃ alkyl, which is optionally substituted with from 1-3 halo (F, Cl, Br, I, preferably F) groups or 1 or 2 hydroxy groups, —O—C₁-C₃-alkyl, which is optionally substituted with from 1-3 halo, preferably F groups, —(CH₂)_{K}COOH, —(CH₂)_{K}C(O)O—C₁-C₄ alkyl which is optionally substituted with from 1-3 halo, preferably F groups, O—C(O)—C₁-C₄ alkyl, which is optionally substituted with from 1-3 halo, preferably F groups or —(CH₂)_{K}C(O)—C₁-C₄ alkyl which is optionally substituted with from 1-3 halo, preferably F groups, or
R^{TA} is a

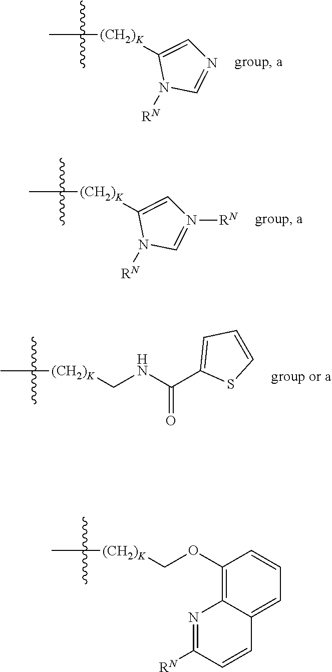

group, or
R^{TA} is a

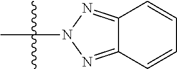 [SMILES: *n1nc2ccccc2n1]

group which is optionally substituted with up to three, preferably 1 C₁-C₃ alkyl groups which are optionally substituted with up to three halo (preferably F) groups, or
R^{TA} is a

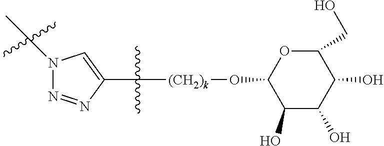

group,
wherein R^{N}, R^{N1} and R^{N2} are each independently H or a C₁-C₃ alkyl group which is optionally substituted with from one to three halo groups, preferably F, or one or two hydroxyl groups and wherein each —(CH₂)_{K} group is optionally substituted with 1-4, preferably 1 or 2, C₁-C₃ alkyl groups which are optionally substituted with from 1-3 fluoro groups or 1-2 hydroxyl groups; and K is independently 0-4 (0, 1, 2, 3 or 4), preferably 0 or 1, or a pharmaceutically acceptable salt, stereoisomer, solvate or polymorph thereof.

In an embodiment,
X is —O—C(R^{N1})(R^{N1}),
C(R^{N1})(R^{N1})—O—, —S—C(R^{N1})(R^{N1}), C(R^{N1})(R^{N1})—S—, N(R^{N1})—C(R^{N1})(R^{N1})
C(R^{N1})(R^{N1})—N(R^{N1}) or C(R^{N1})(R^{N1})—C(R^{N1})(R^{N1}) when X is 2 atoms in length,
X is —O—C(R^{N1})(R^{N1})—C(R^{N1})(R^{N1}), C(R^{N1})(R^{N1})—O—C(R^{N1})(R^{N1},
—O—C(R^{N1})(R^{N1})—O—, —O—C(R^{N1})(R^{N1})—S—, —O—C(R^{N1})(R^{N1})—N(R^{N1})—,
—S—C(R^{N1})(R^{N1})—C(R^{N1})(R^{N1}), C(R^{N1})(R^{N1})—S—C(R^{N1})(R^{N1})—, C(R^{N1})(R^{N1})—(R^{N1})(R^{N1})—S, —S—C(R^{N1})(R^{N1})—S—, —S—C(R^{N1})(R^{N1})—O—, —S—C(R^{N1})(R^{N1})—N(R^{N1})—, N(R^{N1})—C(R^{N1})(R^{N1})—C(R^{N1})(R^{N1}), C(R^{N1})(R^{N1})—N(R^{N1})—C(R^{N1})(R^{N1}) C(R^{N1})(R^{N1})—C(R^{N1})(R^{N1})—N(R^{N1}), N(R^{N1})—C(R^{N1})(R^{N1})—N(R^{N1}) or C(R^{N1})(R^{N1})—C(R^{N1})(R^{N1})—C(R^{N1})(R^{N1}) when X is 3 atoms in length, and
X is-O—C(R^{N1})(R^{N1})—C(R^{N1})(R^{N1})—C(R^{N1})(R^{N1}), C(R^{N1})(R^{N1})—O—C(R^{N1})(R^{N1})—(R^{N1})(R^{N1})—, —O—C(R^{N1})(R^{N1})—O—C(R^{N1})(R^{N1})—, —S—C(R^{N1})(R^{N1})—C(R^{N1})(R^{N1})—C(R^{N1})(R^{N1})—, C(R^{N1})(R^{N1})—S—C(R^{N1})(R^{N1})—C(R^{N1})(R^{N1})—, C(R^{N1})(R^{N1})—(R^{N1})(R^{N1})—S—C(R^{N1})(R^{N1})—, —S—C(R^{N1})(R^{N1})—S—C(R^{N1})(R^{N1})—, N(R^{N1})—C(R^{N1})(R^{N1})—C(R^{N1})(R^{N1})—C(R^{N1})(R^{N1})—/C(R^{N1})(R^{N1})—N(R^{N1})—C(R^{N1})(R^{N1})—C(R^{N1})(R^{N1}), C(R^{N1})(R^{N1})—C(R^{N1})(R^{N1})—N(R^{N1}), N(R^{N1})—C(R^{N1})(R^{N1})—N(R^{N1}) or C(R^{N1})(R^{N1})—C(R^{N1})(R^{N1})—C(R^{N1})(R^{N1}) when X is 4 atoms in length,
wherein R^{N1} is the same as set forth in claim 4 or 6 above.

In an embodiment, X is OCH₂ or CH₂O and R^{N1} is H.

The cellular receptor binding moiety may include an ASGPR binding group according to the chemical structure:

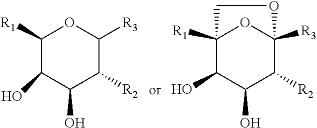

where R₁, R₂ and R₃ are the same as in claim 9, or a pharmaceutically acceptable salt, stereoisomer, solvate or polymorph thereof.

The cellular receptor binding moiety may have the following structure:

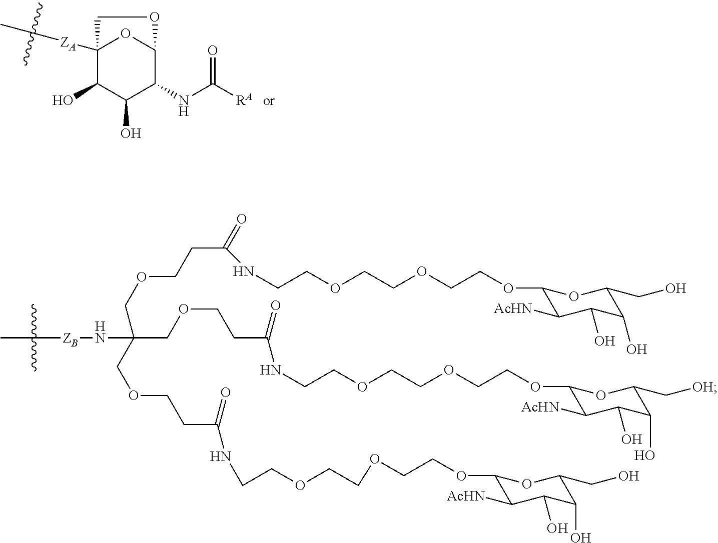

where R^{A} is a C₁-C₃ alkyl group which is optionally substituted with 1-5 halo (preferably fluoro) groups (preferably R^{A} is a methyl or ethyl group which is optionally substituted with from 1-3 fluoro groups);
Z_{A} is —(CH₂)_{IM}, —O—(CH₂)_{IM}, S—(CH₂)_{IM}, NR_{M}—(CH₂)_{IM}, C(O)—(CH₂)_{IM}—, a PEG group containing from 1 to 8 preferably 1-4 ethylene glycol residues or a —C(O)(CH₂)_{IM}NR_{M} group (preferably a PEG containing group comprising from 1 to 8 ethylene glycol, preferably 2-4 ethylene glycol residues) where IM and R_{M} are the same as above; and
Z_{B} is absent, (CH₂)_{IM}, C(O)—(CH₂)_{IM}— or C(O)—(CH₂)_{IM}—NR_{M}, where IM and R_{M} are the same as above.

In an embodiment, R^{A} may be a methyl or ethyl group which is optionally substituted with from 1-3 fluoro groups.

In an embodiment, Z_{A} may be a PEG group containing from 1 to 4 ethylene glycol residues.

In an embodiment, the methyl or ethyl group may be substituted with from 1-3 fluoro groups.

In an embodiment, the ASGPR binding group may be N-acetyl-D-galactosamine.

In an embodiment, the cellular receptor binding moiety may be a low density lipoprotein receptor-related protein 1 (LRP1), a low density lipoprotein receptor (LDLR), a FcγRI binding group, a FcRN binding group, a transferrin receptor binding group, or a macrophage scavenger receptor binding group.

The invention is further illustrated by non-limited examples.

### EXAMPLES

Proper protein section and turnover is a necessary process for maintaining homeostasis. Newly synthesized proteins targeted for secretion are first trafficked to the endoplasmic reticulum, where they are post-translationally modified with N-linked glycan chains terminating in sialic acids. As proteins age, terminal sialic acid residues are removed by circulating endogenous glycosydases. This natural protein aging process unmasks galactose and N-acetylgalactose (GaINAc) residues, which bind the asialoglycoprotein receptor (ASGPR) on the surface of hepatocytes. The ASGPR is a C-type lectin that removes aged circulating proteins with exposed GaINAc residues from circulation by trafficking them to lysosomes. Multiple galactose or GaINAc residues displayed on the protein surface are necessary for high-affinity binding to and subsequent endocytosis by ASGPR. Once these proteins are endocytosed, they are released from the ASGPR through depletion of calcium from the endosome and changes in binding site amino acid protonation changes due to a decrease in pH; the ASGPR is recycled back to the hepatocyte surface. Endocytosed proteins are trafficked to late endosomes, which are fused with lysosomes. Lysosomal proteases then degrade endocytosed proteins, permanently removing them from circulation (M).

Non-glycosylated proteins (e.g., immunoglobulins) are not known to be natural target for the ASGPR. One such protein is galactose-deficient IgA1 (Gd-IgAQ1), which is shown to be implicated in pathophysiology of IgA nephropathy. We propose a bifunctional molecule for degrading circulating Gd-IgAQ1 that takes advantage of ASGPR as an entryway for proteins into the endosomal-lysosomal degradation pathway.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A compound having the structure of formula A-I: 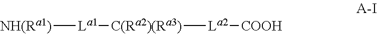
or a salt thereof, wherein
R^{a1} is hydrogen or a protective group;
L^{a1}-CH₂CH₂S—, wherein the CH₂ is bonded to NH(R^{a1});
R^{a2} and R^{a3} are independently hydrogen or a group selected from 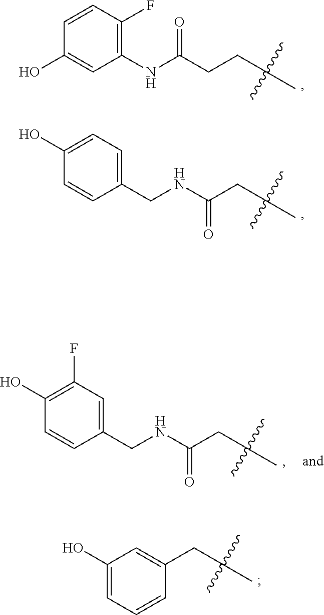
and
L^{a2} is a covalent bond or a group selected from

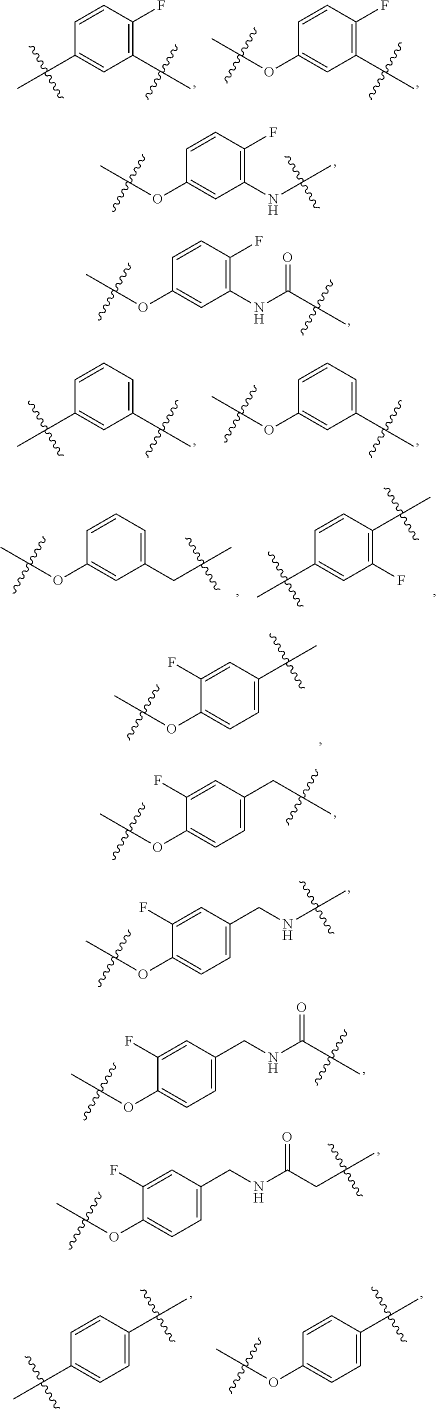

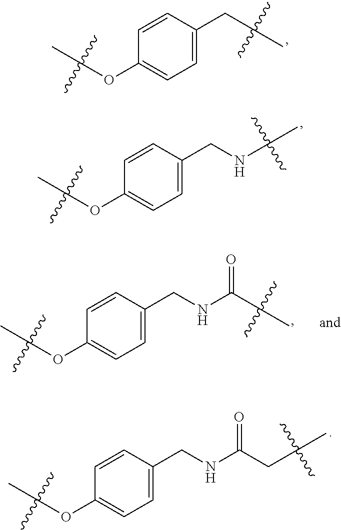 [SMILES: *Cc1ccc(O*)cc1 ; *NCc1ccc(O*)cc1 ; *Oc1ccc(CNC(*)=O)cc1 ; *CC(=O)NCc1ccc(O*)cc1]

2. The compound of claim 1, wherein R^{a1} is hydrogen.

3. The compound of claim 1, wherein R^{a1} is a protective group selected from Fmoc and Dde.

4. The compound of claim 1, wherein at least one selected from R^{a2} and R^{a3} is hydrogen.

5. The compound of claim 1, wherein L^{a2} is a covalent bond and only one selected from R^{a2} and R^{a3} is hydrogen.

6. The compound of claim 1, wherein both R^{a2} and R^{a3} is hydrogen, and L^{a2} is a group selected from

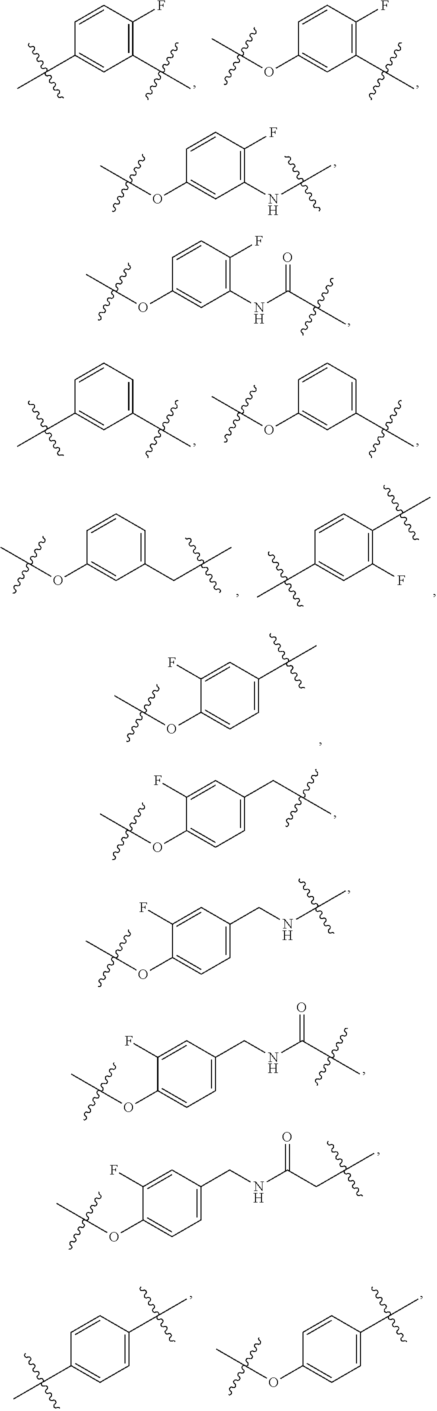

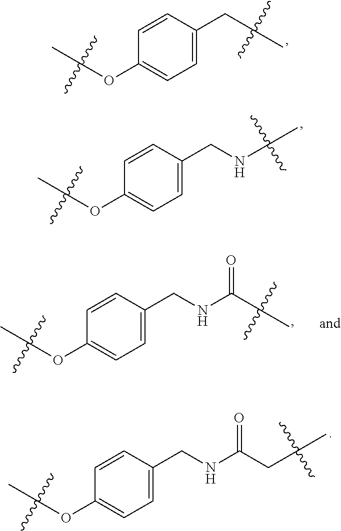 [SMILES: *Cc1ccc(O*)cc1 ; *NCc1ccc(O*)cc1 ; *Oc1ccc(CNC(*)=O)cc1 ; *CC(=O)NCc1ccc(O*)cc1]

7. The compound of claim 1, wherein L^{a2} is a group selected from

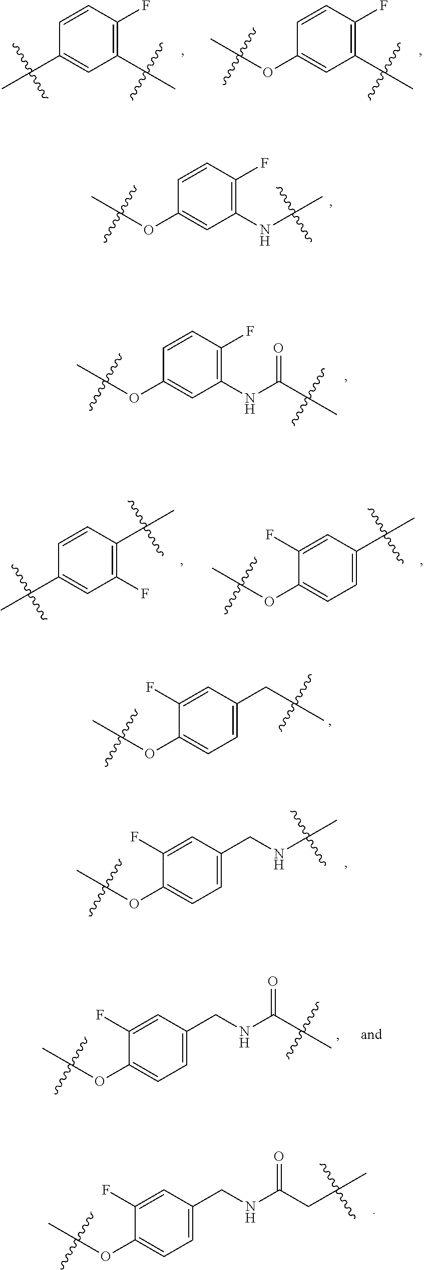

8. The compound of claim 7, wherein L^{a2} is a group selected from

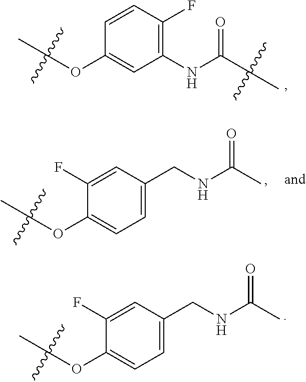 [SMILES: *Oc1ccc(F)c(NC(*)=O)c1 ; "," ; *Oc1ccc(CNC(C)=O)cc1F ; "," ; *Oc1ccc(CNC(C)=O)cc1F ; "."]

9. The compound of claim 1, wherein L^{a2} is a covalent bond, R^{a2} is hydrogen, and R^{a3} is a group selected from

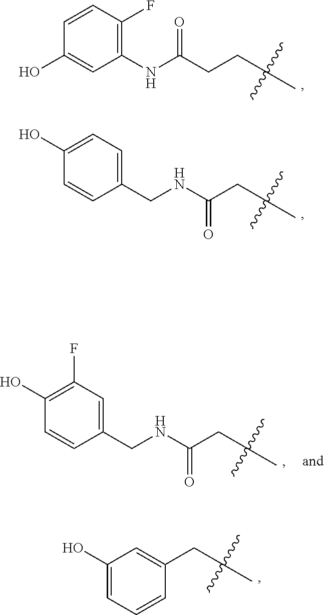

10. The compound of claim 9, wherein R^{a3} is a group selected from H and 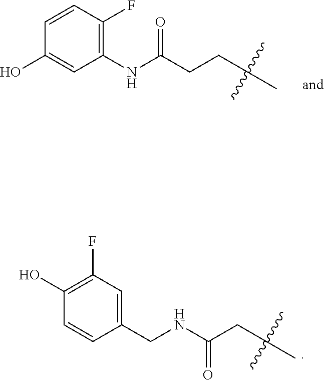 [SMILES: *CCC(=O)Nc1cc(O)ccc1F ; *CC(=O)NCc1ccc(O)c(F)c1 ; "."]
